SEQUENCE LISTING (82 sequences)

SEQ ID NO: 1 - protein, 500 aa, synthetic construct
MWRPSDSTVYVPPPNPVSKVVATDAYVTRTNIFYHASSSRLLAVGHPYFSIKRANKTVVPKVSGYQYRVFKVVLPDPNKFALPDSSLFDPTTQRLVWACTGLEVGRGQPLGVGVSGHPFLNKYDDVENSGSGGNPGQDNRVNVGMDYKQTQLCMVGCAPPLGEHWGKGKQCTNTPVQAGDCPPLELITSVIQDGDMVDTGFGAMNFADLQTNKSDVPIDICGTTCKYPDYLQMAADPYGDRLFFFLRKEQMFARHFFNRAGEVGEPVPDTLIIKGSGNRTSVGSSIYVNTPSGSLVSSEAQLFNKPYWLQKAQGHNNGICWGNQLFVTVVDTTRSTNMTLCASVTTSSTYTNSDYKEYMRHVEEYDLQFIFQLCSITLSAEVMAYIHTMNPSVLEDWNFGLSPPPNGTLEDTYRYVQSQAITCQKPTPEKEKPDPYKNLSFWEVNLKEKFSSELDQYPLGRKFLLQSGYRGRSSIRTGVKRPAVSKASAAPKRKRAKTKR

SEQ ID NO: 2 - protein, 505 aa, synthetic construct
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKL

SEQ ID NO: 3 - protein, 507 aa, synthetic construct
MALWRPSDNTVYLPPPSVARVVNTDDYVTPTSIFYHAGSSRLLTVGNPYFRVPAGGGNKQDIPKVSAYQYRVFRVQLPDPNKFGLPDTSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQAGLRRKPTIGPRKRSAPSATTSSKPAKRVRVRARK

SEQ ID NO: 4 - protein, 504 aa, synthetic construct
MSLWRPSEATVYLPPVPVSKVVSTDEYVTRTNIYYHAGSARLLTVGHPYYSIPKSDNPKKIVVPKVSGLQYRVFRVRLPDPNKFGFPDTSFYNPETQRLVWACVGLEVGRGQPLGVGISGHPLLNKFDDTENSNRYAGGPGTDNRECISMDYKQTQLCLLGCKPPIGEHWGKGSPCSNNAITPGDCPPLELKNSVIQDGDMVDTGFGAMDFTALQDTKSNVPLDICNSICKYPDYLKMVAEPYGDTLFFYLRREQMFVRHFFNRSGTVGESVPTDLYIKGSGSTATLANSTYFPTPSGSMVTSDAQIFNKPYWMQRAQGHNNGICWGNQLFVTVVDTTRSTNMSVCAAIANSDTTFKSSNFKEYLRHGEEFDLQFIFQLCKITLSADIMTYIHSMNPAILEDWNFGLTTPPSGSLEDTYRFVTSQAITCQKTAPQKPKEDPFKDYVFWEVNLKEKFSADLDQFPLGRKFLLQAGYRARPKFKAGKRSAPSASTTTPAKRKKTKK

SEQ ID NO: 5 - protein, 499 aa, synthetic construct
MSVWRPSEATVYLPPVPVSKVVSTDEYVSRTSIYYYAGSSRLLAVGHPYFSIKNPTNAKKLLVPKVSGLQYRVFRVRLPDPNKFGFPDTSFYNPDTQRLVWACVGLEIGRGQPLGVGISGHPLLNKFDDTETGNKYPGQPGADNRECLSMDYKQTQLCLLGCKPPTGEHWGKGVACTNAAPANDCPPLELINTIIEDGDMVDTGFGCMDFKTLQANKSDVPIDICGSTCKYPDYLKMTSEPYGDSLFFFLRREQMFVRHFFNRAGTLGEAVPDDLYIKGSGTTASIQSSAFFPTPSGSMVTSESQLFNKPYWLQRAQGHNNGICWGNQVFVTVVDTTRSTNMTLCTQVTSDSTYKNENFKEYIRHVEEYDLQFVFQLCKVTLTAEVMTYIHAMNPDILEDWQFGLTPPPSASLQDTYRFVTSQAITCQKTVPPKEKEDPLGKYTFWEVDLKEKFSADLDQFPLGRKFLLQAGLKAKPKLKRAAPTSTRTSSAKRKKVKK

SEQ ID NO: 6 - protein, 502 aa, synthetic construct
MSLWRSNEATVYLPPVSVSKVVSTDEYVTRTNIYYHAGSSRLLAVGHPYYAIKKQDSNKIAVPKVSGLQYRVFRVKLPDPNKFGFPDTSFYDPASQRLVWACTGVEVGRGQPLGVGISGHPLLNKLDDTENSNKYVGNSGTDNRECISMDYKQTQLCLIGCRPPIGEHWGKGTPCNANQVKAGECPPLELLNTVLQDGDMVDTGFGAMDFTTLQANKSDVPLDICSSICKYPDYLKMVSEPYGDMLFFYLRREQMFVRHLFNRAGTVGETVPADLYIKGTTGTLPSTSYFPTPSGSMVTSDAQIFNKPYWLQRAQGHNNGICWSNQLFVTVVDTTRSTNMSVCSAVSTSDSTYKNDNFKEYLRHGEEYDLQFIFQLCKITLTADVMTYIHSMNPSILEDWNFGLTPPPSGTLEDTYRYVTSQAVTCQKPSAPKPKDDPLKNYTFWEVDLKEKFSADLDQFPLGRKFLLQAGLKARPNFRLGKRAAPASTSKKSSTKRRKVKS

SEQ ID NO: 7 - protein, 539 aa, synthetic construct
MAHNIIYGHGIIIFLKNVNVFPIFLQMALWRPSDSTVYLPPPSVARVVSTDDYVSRTSIFYHAGSSRLLTVGNPYFRVVPNGAGNKQAVPKVSAYQYRVFRVALPDPNKFGLPDSTIYNPETQRLVWACVGMEIGRGQPLGIGLSGHPFYNKLDDTESAHAATAVITQDVRDNVSVDYKQTQLCILGCVPAIGEHWAKGTLCKPAQLQPGDCPPLELKNTIIEDGDMVDTGYGAMDFSTLQDTKCEVPLDICQSICKYPDYLQMSADPYGDSMFFCLRREQLFARHFWNRAGVMGDTVPTDLYIKGTSANMRETPGSCVYSPSPSGSIITSDSQLFNKPYWLHKAQGHNNGICWHNQLFVTVVDTTRSTNLTLCASTQNPVPSTYDPTKFKQYSRHVEEYDLQFIFQLCTITLTAEVMSYIHSMNSSILENWNFGVPPPPTTSLVDTYRFVQSVAVTCQKDTTPPEKQDPYDKLKFWTVDLKEKFSSDLDQYPLGRKFLVQAGLRRRPTIGPRKRPAASTSTASTASRPAKRVRIRSKK

SEQ ID NO: 8 - protein, 529 aa, synthetic construct
MVQILFYILVIFYYVAGVNVFHIFLQMSVWRPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKDYMFWEVDLKEKFSADLDQFPLGRKFLLQAGLQARPKLKRPASSAPRTSTKKKKVKR

SEQ ID NO: 9 - protein, 524 aa, synthetic construct
MVLILCCTLAILFCVADVNVFHIFLQMSVWRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKTAPPKEKEDPLNKYTFWEVNLKEKFSADLDQFPLGRKFLLQSGLKAKPRLKRSAPTTRAPSTKRKKVKK

SEQ ID NO: 10 - protein, 501 aa, synthetic construct
MWRPSDSTVYVPPPNPVSKVVATDAYVKRTNIFYHASSSRLLAVGHPYYSIKKVNKTVVPKVSGYQYRVFKVVLPDPNKFALPDSSLFDPTTQRLVWACTGLEVGRGQPLGVGVSGHPLLNKYDDVENSGGYGGNPGQDNRVNVGMDYKQTQLCMVGCAPPLGEHWGKGTQCSNTSVQNGDCPPLELITSVIQDGDMVDTGFGAMNFADLQTNKSDVPLDICGTVCKYPDYLQMAADPYGDRLFFYLRKEQMFARHFFNRAGTVGEPVPDDLLVKGGNNRSSVASSIYVHTPSGSLVSSEAQLFNKPYWLQKAQGHNNGICWGNHLFVTVVDTTRSTNMTLCASVSKSATYTNSDYKEYMRHVEEFDLQFIFQLCSITLSAEVMAYIHTMNPSVLEDWNFGLSPPPNGTLEDTYRYVQSQAITCQKPTPEKEKQDPYKDMSFWEVNLKEKFSSELDQFPLGRKFLLQSGYRGRTSARTGIKRPAVSKPSTAPKRKRTKTKK

SEQ ID NO: 11 - DNA, 1503 bp, synthetic construct
  source 1..1503: mol_type = other DNA
atgtggcgtccctccgactccactgtttatgttccaccaccaaatccagtctccaaggttgttgctactgacgcctacgttaccaggaccaatattttctaccacgcctcctcatccagattgctggctgttggtcatccatacttctccatcaagagagccaacaagaccgtcgttccaaaggtttctggttaccagtacagagtgttcaaggtcgtcttgccagatccaaacaagttcgctttgccagactcttccttgttcgacccaactactcagagattggtttgggcttgtaccggtttggaagttggtagaggtcaaccacttggtgttggtgtttctggtcacccattcttgaacaagtacgacgacgttgaaaactccggttctggtggtaacccaggtcaagacaacagagttaacgtcggtatggactacaagcagacccagttgtgtatggttggttgtgctccaccattgggtgaacattggggtaagggtaagcagtgtaccaacactccagttcaagctggtgattgtccacctttggagttgatcacttccgttattcaggacggtgacatggttgacactggtttcggtgctatgaacttcgctgacttgcagactaacaagtccgacgttcctatcgacatctgtggtactacctgtaagtacccagactacttgcaaatggctgctgatccatacggtgacagactgttcttcttcctgagaaaagaacagatgttcgccagacacttcttcaacagagctggtgaagttggtgaaccagttccagacaccttgatcatcaaaggttccggtaacagaacctccgttggttcctctatctacgttaacactccatccggttctttggtgtcctctgaagctcagttgttcaacaagccatactggttgcaaaaggcccagggtcacaacaacggtatctgttggggtaaccagttgttcgttactgttgtcgacactaccagatccaccaatatgactttgtgtgcctccgttactacctcctccacttacactaactccgactacaaagagtacatgagacacgtcgaagagtacgacctgcagttcattttccagctgtgttccatcactttgtccgctgaggttatggcctacatccacactatgaacccatccgttttggaggactggaacttcggtttgtctccaccacctaacggtactttggaggatacctacagatacgttcagtcccaggctatcacttgccaaaagccaactccagagaaagagaagccagatccttacaagaacctgtccttctgggaagtcaacctgaaagaaaagttctccagcgagctggatcagtacccattgggtagaaagttcttgctgcagtccggttacagaggtagatcctccattagaaccggtgttaagaggcctgctgtttctaaggcttctgctgctccaaagaggaagagagctaagaccaagagatag

SEQ ID NO: 12 - DNA, 1518 bp, synthetic construct
  source 1..1518: mol_type = other DNA
atgtccctgtggctgccatccgaggctactgtttatttgccaccagttccagtctccaaggttgtttctactgacgagtacgtcgccaggaccaacatctactatcatgctggtacttccagattgctggctgttggtcatccatacttcccaatcaagaagccaaacaacaacaagatcctggtgccaaaggtttccggtctgcagtacagagttttcagaatccacttgccagatccaaacaagttcggtttcccagacacatccttctacaacccagacactcagagattggtttgggcctgtgttggtgttgaagttggtagaggtcaaccacttggtgttggtatttctggtcacccattgctgaacaagttggacgatactgaaaacgcttccgcttacgctgctaacgctggtgttgataacagagaatgcatctccatggactacaagcagacccagttgtgtttgatcggttgcaagccacctattggtgaacattggggtaagggttctccatgtaccaacgttgctgttaacccaggtgattgtccaccattggagctgatcaacactgttattcaggacggtgacatggtcgacactggttttggtgctatggacttcactaccttgcaggctaacaagtccgaagttccattggacatctgtacctccatctgcaagtacccagactacatcaagatggtgtctgagccatacggtgactccctgttcttctacttgagaagagaacagatgttcgtcaggcacctgttcaacagagctggtgctgttggtgaaaacgttccagacgacctgtacatcaaaggttctggttctactgctaacctggcctcctctaactactttccaactccatccggttccatggttacttccgacgctcagattttcaacaagccatactggttgcaaagagcccagggtcacaacaacggtatctgttggggtaaccagttgttcgttactgttgtcgacactaccagatccaccaacatgtctttgtgtgctgctatctccacttccgagactacctacaagaacactaacttcaaagaatacctgaggcacggtgaagagtacgacttgcagttcattttccagctgtgcaagatcactttgaccgccgacgttatgacttacatccactccatgaactccactatcttggaggattggaactttggtctgcaaccaccaccaggtggtactttggaagatacctacagattcgttacttcccaggctatcgcttgtcagaagcacactccaccagctccaaaagaggatccattgaagaagtacactttctgggaagtcaacctgaaagagaagttctccgctgacttggatcagttcccactgggtagaaagttcttgttgcaggctggtttgaaggccaagccaaagtttaccttgggtaagagaaaggctactccaactacttcctccacctctactaccgctaagaggaagaagagaaagttgtag

SEQ ID NO: 13 - DNA, 1524 bp, synthetic construct
  source 1..1524: mol_type = other DNA
atggccctgtggcgtccatccgacaacactgtttatttgccaccaccatccgttgccagagttgttaacactgacgactacgttactcccacctccattttctaccacgctggttcttctagattgctgaccgttggtaacccatacttcagagttccagctggtggtggtaacaagcaggacattccaaaggtttccgcctaccagtacagagtgttcagagtccaattgccagatccaaacaagttcggtttgccagacacctccatctacaacccagagactcaaagattggtttgggcttgtgctggtgtcgagattggtagaggtcaaccacttggtgttggtttgtctggtcacccattctacaacaagttggacgacactgaatcttcccacgctgctacttctaacgtttccgaggatgtcagagacaacgtgtccgttgactacaagcagacccagttgtgtatcttgggttgtgctccagctattggtgaacattgggctaagggaactgcctgtaagtccagaccattgtctcaaggtgattgcccaccattggagttgaagaacaccgttttggaggacggtgacatggttgatactggttacggtgctatggacttctccaccttgcaagacactaagtgtgaggtcccattggacatctgtcagtccatctgtaagtacccagactacttgcagatgtccgctgatccatacggtgactctatgttcttctgcctgagaagagagcagttgttcgctagacacttctggaacagagctggtactatgggtgacactgttccacagtccttgtacatcaaaggtactggtatgagagcttccccaggttcctgtgtttactctccatctccatccggttccatcgttacttccgattctcagctgttcaacaagccatactggttgcacaaggctcagggtcataacaacggtgtttgttggcacaaccagctgttcgttactgttgttgacactaccagatccaccaacttgactatctgtgcttccactcaatccccagttccaggtcaatacgacgctactaagttcaagcagtactccagacacgtcgaagagtacgacttgcagttcatcttccagttgtgcactatcactttgaccgccgacgttatgtcctacatccactctatgaactcctccatcttggaggactggaactttggtgttcctcctcctccaactacttccttggttgacacctacagattcgttcagtccgtcgctatcacttgtcaaaaggatgctgctcctgctgagaacaaggacccatacgataagctgaagttctggaacgtcgacctgaaagagaagttctccttggacttggatcagtacccactgggtagaaagttcttggtccaagccggtttgagaagaaagccaactatcggtccaagaaagagatctgctccttccgctactacttcttccaagccagctaagagagttagagtcagagccagaaagtag

SEQ ID NO: 14 - DNA, 1515 bp, synthetic construct
  source 1..1515: mol_type = other DNA
atgtctttgtggcgtccatctgaggctaccgtttacttgccaccagttccagtttccaaggttgtttccactgacgagtacgtcaccaggaccaacatctactaccatgctggttctgctagattgctgactgttggtcacccttactactccattccaaagtctgacaaccccaagaagatcgtcgttccaaaggtttccggactgcagtacagagtgttcagagtcagattgccagatccaaacaagttcggtttccccgacacatccttctacaacccagagactcaaagattggtttgggcctgtgttggtttggaggttggtagaggtcaaccacttggtgttggtatttccggtcatccactgctgaacaagtttgacgacactgagaactccaacagatacgctggtggtccaggtactgacaacagagaatgtatctccatggactacaagcagacccagttgtgtttgttgggttgcaagccacctattggtgaacattggggtaagggttctccatgttccaacaacgctattaccccaggtgattgtccaccattggagttgaagaactccgttattcaggacggtgacatggttgacactggtttcggtgctatggatttcactgccttgcaagacaccaagtccaacgttccattggacatctgtaactccatctgcaagtacccagactacttgaagatggttgctgagccatacggtgacaccttgttcttctacctgagaagagaacagatgttcgtcagacacttcttcaacagatccggtactgttggtgagtccgttccaactgacctgtacatcaaaggttctggttccactgctactttggccaactctacttacttcccaactccatccggttccatggttacttccgacgctcagattttcaacaagccatactggatgcaaagagcccagggtcacaacaacggtatttgttggggtaaccagttgttcgttaccgttgtcgacactaccagatccaccaacatgtctgtttgtgctgctatcgctaactccgacaccactttcaagtcctccaacttcaaagaatacctgagacacggtgaagagttcgacctgcagttcattttccagctgtgcaagatcactttgtccgccgacatcatgacctacatccactctatgaacccagccatcttggaggattggaacttcggtttgactactccaccatctggatccttggaggacacctacagattcgttacttctcaggccatcacctgtcaaaagactgctccacaaaagccaaaagaggacccattcaaggactacgtgttctgggaagtcaacttgaaagagaagttctccgctgacctggatcagttcccattgggtagaaagttcttgttgcaggctggttacagagccagaccaaagtttaaggctggtaagagatctgctccttccgcttctactactaccccagccaagagaaagaaaactaagaagtaa

SEQ ID NO: 15 - DNA, 1500 bp, synthetic construct
  source 1..1500: mol_type = other DNA
atgtctgtttggaggccatctgaggctaccgtttacttgccaccagttccagtttccaaggttgtttccactgacgagtacgtttccagaacctccatctactactacgctggttcttccagattgctggctgttggtcacccatacttctccatcaagaacccaactaacgccaagaagttgctggtcccaaaggtttctggattgcagtacagagtgttcagagtcagattgccagatccaaacaagttcggtttccccgacacatccttctacaacccagacactcaaagattggtttgggcctgtgttggtttggagattggtagaggtcaaccacttggtgttggtatctctggtcaccctttgctgaacaagtttgacgacactgagactggtaacaagtacccaggtcaacctggtgctgacaacagagaatgtctgtctatggactacaagcagacccagttgtgtttgttgggttgtaagccaccaactggtgaacattggggtaagggtgttgcttgtactaatgctgctccagctaacgactgtccaccattggagttgattaacaccatcatcgaggacggtgacatggttgatactggtttcggttgcatggacttcaagaccttgcaagctaacaagtccgacgtgccaattgacatctgtggttccacttgtaagtaccccgactacttgaagatgacctctgagccatacggtgactccctgttctttttcttgcgtcgtgaacagatgttcgtcagacacttcttcaacagagccggtactttgggtgaagctgttccagacgacctgtacatcaaaggttctggtactaccgcttccattcagtcctctgctttcttcccaactccatccggttctatggttacttctgagtcccagctgttcaacaagccatactggttgcaaagagcccagggtcacaacaacggtatctgttggggtaaccaggttttcgttactgttgtcgacactaccagatccaccaacatgactttgtgtactcaggttacttccgactccacctacaagaacgagaacttcaaagagtacatcaggcacgtcgaagagtacgacttgcagttcgttttccagttgtgcaaggtcactttgaccgctgaggttatgacttacatccacgctatgaaccccgacattttggaggattggcaattcggtttgactccaccaccatctgcttctctgcaagacacctacagattcgttacttcccaggccatcacctgtcaaaagaccgttccaccaaaagagaaagaggacccattgggcaagtacactttctgggaagttgacctgaaagagaagttctccgctgacttggatcagttcccactgggtagaaagttcttgttgcaggctggtttgaaggccaagccaaagttgaaaagagctgctccaacctccactagaacctcttccgctaaaagaaagaaggtcaagaagtaa

SEQ ID NO: 16 - DNA, 1509 bp, synthetic construct
  source 1..1509: mol_type = other DNA
atgtccctttggagatccaacgaggctaccgtttacttgccaccagtttccgtttccaaggttgtttccactgacgagtacgtcaccaggaccaacatctactaccatgctggttcttccagattgctggctgttggtcatccttactacgccattaagaagcaggactccaacaagatcgccgttccaaaggtttctggtctgcagtacagagtgttcagagtcaagttgccagatccaaacaagttcggtttccccgacacttctttctacgaccctgcttctcaaagattggtttgggcttgtaccggtgttgaggttggtagaggtcaaccacttggtgttggtatttctggtcacccattgctgaacaagttggacgacactgagaactctaacaagtacgtcggtaactccggtactgacaacagagaatgtatctccatggactacaagcagacccagttgtgtttgatcggttgcagaccacctattggtgaacattggggtaagggtactccatgtaacgccaaccaggttaaggctggtgaatgtccaccattggagttgttgaacactgtcttgcaggacggtgacatggttgatactggtttcggtgctatggacttcactaccttgcaagctaacaagtccgacgttccattggacatctgttcctccatctgtaagtacccagactacctgaagatggtgtctgaaccatacggcgacatgttgttcttctacctgagaagagaacaaatgttcgtcaggcacctgttcaacagagctggtactgttggtgaaactgttccagccgacttgtacatcaagggtactactggtactttgccctccacctcttacttcccaactccatctggttccatggttacttccgacgctcagatcttcaacaagccatactggttgcaaagagcccagggtcacaacaacggtatctgttggtctaaccagttgttcgttaccgttgtcgacactaccagatccaccaacatgtctgtttgttccgctgtttctacctccgactccacttacaagaacgacaacttcaaagaatacctgagacacggtgaagagtacgacctgcagttcattttccagctgtgcaagatcactttgaccgccgacgttatgacctacatccactctatgaacccatccatcttggaggactggaacttcggtttgactccaccaccatctggaactttggaggacacctacagatacgtcacttcccaggctgttacttgtcaaaagccatctgctccaaagccaaaggacgacccattgaagaactacactttctgggaagtcgacctgaaagaaaagttctccgctgacttggatcagttcccactgggtagaaagttcttgttgcaggctggtttgaaggccagaccaaacttcagattgggtaagagagctgctccagcttccacttctaagaagtcctccaccaagagaagaaaggtcaagtcttaa

SEQ ID NO: 17 - DNA, 1620 bp, synthetic construct
  source 1..1620: mol_type = other DNA
atggcccacaacattatctacggtcacggcatcatcatctttctgaagaacgtcaacgtgttccccatcttcttgcagatggctttgtggcgtccatctgactccactgtttatttgccaccaccatccgttgccagagttgtttctactgacgactacgtttccaggacctccattttctaccacgctggttcttccagattgctgactgttggtaacccatacttcagagtcgttccaaacggtgctggtaacaagcaagctgttccaaaggtttccgcctaccagtacagagttttcagagttgctttgccagatccaaacaagttcggtttgccagactctaccatctacaacccagagactcagagattggtttgggcctgtgttggtatggaaatcggtagaggtcagccacttggtattggtttgtctggtcacccattctacaacaagttggacgacactgaatctgctcacgctgctactgctgttatcactcaagacgtcagagacaacgtttccgtcgactacaagcagacccagttgtgtatcttgggttgtgttccagctattggtgagcattgggctaagggtactttgtgtaagccagctcaattgcagccaggtgattgtccaccattggagttgaagaacaccatcattgaggacggtgacatggttgacactggttacggtgctatggacttctctaccttgcaggacactaagtgtgaggtcccattggacatttgtcagtccatctgtaagtaccccgactacttgcaaatgtccgctgatccatacggtgactctatgttcttctgcctgagaagagagcagttgttcgctagacacttctggaacagagctggtgttatgggtgacactgttccaaccgacctgtacattaagggaacttccgctaacatgagagaaaccccaggttcctgtgtttactctccatctccatccggttccatcatcacttccgattctcagctgttcaacaagccatactggttgcacaaggctcagggtcacaacaacggtatctgttggcacaaccagctgttcgttactgttgttgacactaccagatccaccaacttgactttgtgtgcctccactcaaaacccagttccatctacttacgaccccaccaagttcaagcaatactccagacacgttgaagagtacgacctgcagttcatcttccagttgtgcactatcactttgaccgccgaggttatgtcctacatccactctatgaactcctccatcttggagaactggaacttcggtgttcctcctcctccaactacttccttggttgatacctacagattcgttcagtccgttgccgttacttgtcagaaggacactactccaccagagaagcaagacccatacgacaagttgaagttctggaccgtcgacctgaaagagaagttctcctctgacttggatcaatacccactgggtagaaagttcttggtccaggctggtttgagaagaaggccaactatcggtccaagaaaaagaccagctgcttctacttctactgcttccactgcttctagaccagccaagagagttagaatcagatccaagaagtaa

SEQ ID NO: 18 - DNA, 1590 bp, synthetic construct
  source 1..1590: mol_type = other DNA
atggtccagatcctgttctacatcctggtcatcttctactacgttgccggtgtcaacgtgttccacatcttcttgcaaatgtccgtttggaggccatccgaggctactgtttatttgccaccagttccagtctccaaggttgtttctactgacgagtacgtttccaggacctccatctactactatgctggttcttccagattgctgaccgttggtcacccatacttctctatcaagaacacctcttccggtaacggtaagaaggtcttggtcccaaaggtttccggattgcagtacagagtgttcagaatcaagctgccagatccaaacaagttcggtttcccagacacatccttctacaacccagagactcagagattggtttgggcttgtactggtttggagatcggtagaggtcaaccacttggtgttggtatttctggtcaccctctgctgaacaagtttgacgacactgagacttccaacaagtacgctggtaagccaggtatcgacaacagagaatgcctgtctatggactacaagcagacccagttgtgtatcttgggttgcaagccacctattggtgaacattggggtaagggtactccatgcaacaacaactctggtaaccctggtgattgtccaccattgcagttgattaactccgtcattcaggacggtgacatggtcgatactggtttcggttgtatggacttcaacaccttgcaagcttccaagtccgacgtgccaattgacatttgttcctccgtctgtaagtaccccgactacttgcagatggcttctgaaccatacggtgactccctgttctttttcttgcgtcgtgaacagatgttcgtcagacacttcttcaacagagccggtactttgggtgatccagttcctggtgacttgtacattcaaggttccaactccggtaacaccgctactgttcaatcttccgctttcttcccaactccatccggttctatggttacttctgagtcccagctgttcaacaagccatactggttgcaaagagcccagggtcacaacaacggtatctgttggggtaaccagttgttcgttaccgttgtcgacactaccagatccaccaacatgactttgtgtgccgaggtcaagaaagagtccacctacaagaacgagaacttcaaagaatacctgagacacggtgaagagttcgacctgcagttcattttccagctgtgcaagatcactttgaccgccgacgttatgacctacatccacaagatggacgctaccattttggaggactggcaattcggtttgactcctcctccttctgcttccttggaagatacctacagattcgttacttccaccgccatcacctgtcagaagaacactccaccaaagggtaaagaggacccactgaaggattacatgttctgggaagtcgacctgaaagagaagttctctgctgacttggatcagttcccactgggtagaaagttcttgttgcaagctggtctgcaggccagaccaaagttgaaaagacctgcttcttccgctccaagaacttccaccaagaagaagaaggttaagagataa

SEQ ID NO: 19 - DNA, 1575 bp, synthetic construct
  source 1..1575: mol_type = other DNA
atggtcttgatcttgtgttgcaccttggccatcttgttctgtgttgctgacgtcaacgtgttccacatcttcttgcaaatgtccgtttggaggccatccgaggctactgtttatttgccaccagttccagtctccaaggttgtttctactgacgagtacgtttccaggacctccatctactactacgctggttcttctagattgctggccgttggtaacccatacttctccattaagtccccaaacaacaacaagaaggtgctggtcccaaaggtttccggattgcagtacagagtgttcagagtcagattgccagatccaaacaagttcggtttccccgacacatccttctacaacccagacactcaaagattggtttgggcctgtgttggtttggagattggtagaggtcaaccacttggtgttggtgtttctggtcacccatacctgaacaagtttgacgacactgagacttccaacagatacccagctcaaccaggttccgacaacagagaatgtctgtctatggactacaagcagacccagttgtgtctgatcggttgtaagccaccaactggtgaacattggggtaagggtgttgcctgtaacaacaacgctgctgctactgattgtccaccattggagctgttcaactccattattgaggacggtgacatggtcgacactggtttcggttgtatggatttcggtactctgcaggctaacaagtccgacgttccaatcgacatctgtaactccacctgtaagtacccagactacctgaagatggcttctgagccatacggtgattccctgttctttttcttgcgtcgtgaacagatgttcgtcagacacttcttcaacagagccggtaagttgggtgaagctgttccagacgacctgtacatcaaaggttctggtaacaccgctgttattcagtcctctgctttcttcccaactccatccggttctatcgttacttctgagtcccagttgttcaacaagccatactggttgcaaagagcccagggtcacaacaacggtatttgttggggtaaccagctgttcgttactgttgttgacactaccagatccaccaacatgaccttgtgtaccgaggttaccaaagagggaacctacaagaacgacaacttcaaagaatacgtcaggcacgtcgaagagtacgacttgcaattcgttttccagctgtgcaagatcactttgaccgctgagatcatgacctacatccacactatggactccaacatcttggaggactggcaattcggtttgactccaccaccatctgcttctctgcaagacacctacagattcgtcacttcccaggctatcacctgtcaaaagactgctccaccaaaagagaaagaggacccattgaacaagtacactttctgggaagtcaacctgaaagagaagttctccgctgacttggatcagttcccactgggtagaaagttcttgttgcagtccggtttgaaggccaagccaagattgaagagatccgctccaactactagagccccatccactaagaggaagaaggttaagaagtaa

SEQ ID NO: 20 - DNA, 1506 bp, synthetic construct
  source 1..1506: mol_type = other DNA
atgtggcgtccctccgactccactgtttatgttccaccaccaaatccagtctccaaggttgttgctactgacgcctacgtcaagaggaccaatattttctaccacgcctcctcatccagattgctggctgttggtcatccttactactccatcaagaaggtcaacaagaccgtcgttccaaaggtttccggttaccagtacagagtgttcaaggtcgttttgccagatccaaacaagttcgctttgcctgactcttccttgttcgacccaactactcagagattggtttgggcttgtaccggtttggaagttggtagaggtcaaccacttggtgttggtgtttctggtcacccattgctgaacaagtacgacgacgttgaaaactccggtggttacggtggtaacccaggtcaagataacagagtcaacgtcggtatggactacaagcagacccagttgtgtatggttggttgtgctccaccattgggtgaacattggggtaagggtactcagtgttccaacacttccgttcagaacggtgattgtccacctttggagttgatcacctccgttattcaagacggtgacatggttgacactggtttcggtgctatgaacttcgctgacttgcagactaacaagtccgacgttcctttggacatctgtggtactgtctgtaagtacccagactacttgcaaatggctgctgatccatacggtgacagactgttcttctacctgagaaaagaacagatgttcgccagacacttcttcaacagagctggtactgttggtgaaccagttccagacgacttgttggtcaaaggtggtaacaacagatcctccgttgcttcctctatctacgttcacactccatccggttccttggtttcttctgaagcccagttgttcaacaagccatactggttgcaaaaggcccagggtcacaacaacggtatttgttggggtaaccacttgttcgttaccgttgtcgacactaccagatccaccaatatgactttgtgtgcctccgtttccaagtccgctacttacactaactccgactacaaagagtacatgagacacgtcgaagagttcgacctgcagttcattttccagctgtgttccattactttgtccgccgaggttatggcctacatccacactatgaacccatccgttttggaggactggaacttcggtttgtctccaccacctaacggtactttggaggatacctacagatacgttcagtcccaggctatcacttgccaaaagccaactccagagaaagagaagcaggacccatacaaggacatgtctttctgggaagtcaacctgaaagaaaagttctcctcagagctggatcagttcccactgggtagaaagttcttgttgcagtccggttacagaggtagaacttccgctagaactggtatcaagaggcctgctgtttctaagccatctactgccccaaagagaaagcgtaccaagaccaagaaatag

SEQ ID NO: 21 - protein, 498 aa, synthetic construct
MPSDSTVYVPPPNPVSKVVATDAYVTRTNIFYHASSSRLLAVGHPYFSIKRANKTVVPKVSGYQYRVFKVVLPDPNKFALPDSSLFDPTTQRLVWACTGLEVGRGQPLGVGVSGHPFLNKYDDVENSGSGGNPGQDNRVNVGMDYKQTQLCMVGCAPPLGEHWGKGKQCTNTPVQAGDCPPLELITSVIQDGDMVDTGFGAMNFADLQTNKSDVPIDICGTTCKYPDYLQMAADPYGDRLFFFLRKEQMFARHFFNRAGEVGEPVPDTLIIKGSGNRTSVGSSIYVNTPSGSLVSSEAQLFNKPYWLQKAQGHNNGICWGNQLFVTVVDTTRSTNMTLCASVTTSSTYTNSDYKEYMRHVEEYDLQFIFQLCSITLSAEVMAYIHTMNPSVLEDWNFGLSPPPNGTLEDTYRYVQSQAITCQKPTPEKEKPDPYKNLSFWEVNLKEKFSSELDQYPLGRKFLLQSGYRGRSSIRTGVKRPAVSKASAAPKRKRAKTKR

SEQ ID NO: 22 - protein, 496 aa, synthetic construct
MDSTVYVPPPNPVSKVVATDAYVTRTNIFYHASSSRLLAVGHPYFSIKRANKTVVPKVSGYQYRVFKVVLPDPNKFALPDSSLFDPTTQRLVWACTGLEVGRGQPLGVGVSGHPFLNKYDDVENSGSGGNPGQDNRVNVGMDYKQTQLCMVGCAPPLGEHWGKGKQCTNTPVQAGDCPPLELITSVIQDGDMVDTGFGAMNFADLQTNKSDVPIDICGTTCKYPDYLQMAADPYGDRLFFFLRKEQMFARHFFNRAGEVGEPVPDTLIIKGSGNRTSVGSSIYVNTPSGSLVSSEAQLFNKPYWLQKAQGHNNGICWGNQLFVTVVDTTRSTNMTLCASVTTSSTYTNSDYKEYMRHVEEYDLQFIFQLCSITLSAEVMAYIHTMNPSVLEDWNFGLSPPPNGTLEDTYRYVQSQAITCQKPTPEKEKPDPYKNLSFWEVNLKEKFSSELDQYPLGRKFLLQSGYRGRSSIRTGVKRPAVSKASAAPKRKRAKTKR

SEQ ID NO: 23 - protein, 501 aa, synthetic construct
MPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKL

SEQ ID NO: 24 - protein, 496 aa, synthetic construct
MVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKL

SEQ ID NO: 25 - protein, 491 aa, synthetic construct
MVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKL

SEQ ID NO: 26 - protein, 486 aa, synthetic construct
MVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKL

SEQ ID NO: 27 - protein, 503 aa, synthetic construct
MPSDNTVYLPPPSVARVVNTDDYVTPTSIFYHAGSSRLLTVGNPYFRVPAGGGNKQDIPKVSAYQYRVFRVQLPDPNKFGLPDTSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQAGLRRKPTIGPRKRSAPSATTSSKPAKRVRVRARK

SEQ ID NO: 28 - protein, 501 aa, synthetic construct
MDNTVYLPPPSVARVVNTDDYVTPTSIFYHAGSSRLLTVGNPYFRVPAGGGNKQDIPKVSAYQYRVFRVQLPDPNKFGLPDTSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQAGLRRKPTIGPRKRSAPSATTSSKPAKRVRVRARK

SEQ ID NO: 29 - protein, 500 aa, synthetic construct
MPSEATVYLPPVPVSKVVSTDEYVTRTNIYYHAGSARLLTVGHPYYSIPKSDNPKKIVVPKVSGLQYRVFRVRLPDPNKFGFPDTSFYNPETQRLVWACVGLEVGRGQPLGVGISGHPLLNKFDDTENSNRYAGGPGTDNRECISMDYKQTQLCLLGCKPPIGEHWGKGSPCSNNAITPGDCPPLELKNSVIQDGDMVDTGFGAMDFTALQDTKSNVPLDICNSICKYPDYLKMVAEPYGDTLFFYLRREQMFVRHFFNRSGTVGESVPTDLYIKGSGSTATLANSTYFPTPSGSMVTSDAQIFNKPYWMQRAQGHNNGICWGNQLFVTVVDTTRSTNMSVCAAIANSDTTFKSSNFKEYLRHGEEFDLQFIFQLCKITLSADIMTYIHSMNPAILEDWNFGLTTPPSGSLEDTYRFVTSQAITCQKTAPQKPKEDPFKDYVFWEVNLKEKFSADLDQFPLGRKFLLQAGYRARPKFKAGKRSAPSASTTTPAKRKKTKK

SEQ ID NO: 30 - protein, 495 aa, synthetic construct
MPSEATVYLPPVPVSKVVSTDEYVSRTSIYYYAGSSRLLAVGHPYFSIKNPTNAKKLLVPKVSGLQYRVFRVRLPDPNKFGFPDTSFYNPDTQRLVWACVGLEIGRGQPLGVGISGHPLLNKFDDTETGNKYPGQPGADNRECLSMDYKQTQLCLLGCKPPTGEHWGKGVACTNAAPANDCPPLELINTIIEDGDMVDTGFGCMDFKTLQANKSDVPIDICGSTCKYPDYLKMTSEPYGDSLFFFLRREQMFVRHFFNRAGTLGEAVPDDLYIKGSGTTASIQSSAFFPTPSGSMVTSESQLFNKPYWLQRAQGHNNGICWGNQVFVTVVDTTRSTNMTLCTQVTSDSTYKNENFKEYIRHVEEYDLQFVFQLCKVTLTAEVMTYIHAMNPDILEDWQFGLTPPPSASLQDTYRFVTSQAITCQKTVPPKEKEDPLGKYTFWEVDLKEKFSADLDQFPLGRKFLLQAGLKAKPKLKRAAPTSTRTSSAKRKKVKK

SEQ ID NO: 31 - protein, 498 aa, synthetic construct
MSNEATVYLPPVSVSKVVSTDEYVTRTNIYYHAGSSRLLAVGHPYYAIKKQDSNKIAVPKVSGLQYRVFRVKLPDPNKFGFPDTSFYDPASQRLVWACTGVEVGRGQPLGVGISGHPLLNKLDDTENSNKYVGNSGTDNRECISMDYKQTQLCLIGCRPPIGEHWGKGTPCNANQVKAGECPPLELLNTVLQDGDMVDTGFGAMDFTTLQANKSDVPLDICSSICKYPDYLKMVSEPYGDMLFFYLRREQMFVRHLFNRAGTVGETVPADLYIKGTTGTLPSTSYFPTPSGSMVTSDAQIFNKPYWLQRAQGHNNGICWSNQLFVTVVDTTRSTNMSVCSAVSTSDSTYKNDNFKEYLRHGEEYDLQFIFQLCKITLTADVMTYIHSMNPSILEDWNFGLTPPPSGTLEDTYRYVTSQAVTCQKPSAPKPKDDPLKNYTFWEVDLKEKFSADLDQFPLGRKFLLQAGLKARPNFRLGKRAAPASTSKKSSTKRRKVKS

SEQ ID NO: 32 - protein, 530 aa, synthetic construct
MIIIFLKNVNVFPIFLQMALWRPSDSTVYLPPPSVARVVSTDDYVSRTSIFYHAGSSRLLTVGNPYFRVVPNGAGNKQAVPKVSAYQYRVFRVALPDPNKFGLPDSTIYNPETQRLVWACVGMEIGRGQPLGIGLSGHPFYNKLDDTESAHAATAVITQDVRDNVSVDYKQTQLCILGCVPAIGEHWAKGTLCKPAQLQPGDCPPLELKNTIIEDGDMVDTGYGAMDFSTLQDTKCEVPLDICQSICKYPDYLQMSADPYGDSMFFCLRREQLFARHFWNRAGVMGDTVPTDLYIKGTSANMRETPGSCVYSPSPSGSIITSDSQLFNKPYWLHKAQGHNNGICWHNQLFVTVVDTTRSTNLTLCASTQNPVPSTYDPTKFKQYSRHVEEYDLQFIFQLCTITLTAEVMSYIHSMNSSILENWNFGVPPPPTTSLVDTYRFVQSVAVTCQKDTTPPEKQDPYDKLKFWTVDLKEKFSSDLDQYPLGRKFLVQAGLRRRPTIGPRKRPAASTSTASTASRPAKRVRIRSKK

SEQ ID NO: 33 - protein, 523 aa, synthetic construct
MVNVFPIFLQMALWRPSDSTVYLPPPSVARVVSTDDYVSRTSIFYHAGSSRLLTVGNPYFRVVPNGAGNKQAVPKVSAYQYRVFRVALPDPNKFGLPDSTIYNPETQRLVWACVGMEIGRGQPLGIGLSGHPFYNKLDDTESAHAATAVITQDVRDNVSVDYKQTQLCILGCVPAIGEHWAKGTLCKPAQLQPGDCPPLELKNTIIEDGDMVDTGYGAMDFSTLQDTKCEVPLDICQSICKYPDYLQMSADPYGDSMFFCLRREQLFARHFWNRAGVMGDTVPTDLYIKGTSANMRETPGSCVYSPSPSGSIITSDSQLFNKPYWLHKAQGHNNGICWHNQLFVTVVDTTRSTNLTLCASTQNPVPSTYDPTKFKQYSRHVEEYDLQFIFQLCTITLTAEVMSYIHSMNSSILENWNFGVPPPPTTSLVDTYRFVQSVAVTCQKDTTPPEKQDPYDKLKFWTVDLKEKFSSDLDQYPLGRKFLVQAGLRRRPTIGPRKRPAASTSTASTASRPAKRVRIRSKK

SEQ ID NO: 34 - protein, 518 aa, synthetic construct
MIFLQMALWRPSDSTVYLPPPSVARVVSTDDYVSRTSIFYHAGSSRLLTVGNPYFRVVPNGAGNKQAVPKVSAYQYRVFRVALPDPNKFGLPDSTIYNPETQRLVWACVGMEIGRGQPLGIGLSGHPFYNKLDDTESAHAATAVITQDVRDNVSVDYKQTQLCILGCVPAIGEHWAKGTLCKPAQLQPGDCPPLELKNTIIEDGDMVDTGYGAMDFSTLQDTKCEVPLDICQSICKYPDYLQMSADPYGDSMFFCLRREQLFARHFWNRAGVMGDTVPTDLYIKGTSANMRETPGSCVYSPSPSGSIITSDSQLFNKPYWLHKAQGHNNGICWHNQLFVTVVDTTRSTNLTLCASTQNPVPSTYDPTKFKQYSRHVEEYDLQFIFQLCTITLTAEVMSYIHSMNSSILENWNFGVPPPPTTSLVDTYRFVQSVAVTCQKDTTPPEKQDPYDKLKFWTVDLKEKFSSDLDQYPLGRKFLVQAGLRRRPTIGPRKRPAASTSTASTASRPAKRVRIRSKK

SEQ ID NO: 35 - protein, 509 aa, synthetic construct
MPSDSTVYLPPPSVARVVSTDDYVSRTSIFYHAGSSRLLTVGNPYFRVVPNGAGNKQAVPKVSAYQYRVFRVALPDPNKFGLPDSTIYNPETQRLVWACVGMEIGRGQPLGIGLSGHPFYNKLDDTESAHAATAVITQDVRDNVSVDYKQTQLCILGCVPAIGEHWAKGTLCKPAQLQPGDCPPLELKNTIIEDGDMVDTGYGAMDFSTLQDTKCEVPLDICQSICKYPDYLQMSADPYGDSMFFCLRREQLFARHFWNRAGVMGDTVPTDLYIKGTSANMRETPGSCVYSPSPSGSIITSDSQLFNKPYWLHKAQGHNNGICWHNQLFVTVVDTTRSTNLTLCASTQNPVPSTYDPTKFKQYSRHVEEYDLQFIFQLCTITLTAEVMSYIHSMNSSILENWNFGVPPPPTTSLVDTYRFVQSVAVTCQKDTTPPEKQDPYDKLKFWTVDLKEKFSSDLDQYPLGRKFLVQAGLRRRPTIGPRKRPAASTSTASTASRPAKRVRIRSKK

SEQ ID NO: 36 - protein, 499 aa, synthetic construct
MPSEATVYLPPVPVSKVVSTDEYVSRTSIYYYAGSSRLLTVGHPYFSIKNTSSGNGKKVLVPKVSGLQYRVFRIKLPDPNKFGFPDTSFYNPETQRLVWACTGLEIGRGQPLGVGISGHPLLNKFDDTETSNKYAGKPGIDNRECLSMDYKQTQLCILGCKPPIGEHWGKGTPCNNNSGNPGDCPPLQLINSVIQDGDMVDTGFGCMDFNTLQASKSDVPIDICSSVCKYPDYLQMASEPYGDSLFFFLRREQMFVRHFFNRAGTLGDPVPGDLYIQGSNSGNTATVQSSAFFPTPSGSMVTSESQLFNKPYWLQRAQGHNNGICWGNQLFVTVVDTTRSTNMTLCAEVKKESTYKNENFKEYLRHGEEFDLQFIFQLCKITLTADVMTYIHKMDATILEDWQFGLTPPPSASLEDTYRFVTSTAITCQKNTPPKGKEDPLKDYMFWEVDLKEKFSADLDQFPLGRKFLLQAGLQARPKLKRPASSAPRTSTKKKKVKR

SEQ ID NO: 37 - protein, 515 aa, synthetic construct
MILFCVADVNVFHIFLQMSVWRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKTAPPKEKEDPLNKYTFWEVNLKEKFSADLDQFPLGRKFLLQSGLKAKPRLKRSAPTTRAPSTKRKKVKK

SEQ ID NO: 38 - protein, 508 aa, synthetic construct
MVNVFHIFLQMSVWRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKTAPPKEKEDPLNKYTFWEVNLKEKFSADLDQFPLGRKFLLQSGLKAKPRLKRSAPTTRAPSTKRKKVKK

SEQ ID NO: 39 - protein, 503 aa, synthetic construct
MIFLQMSVWRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKTAPPKEKEDPLNKYTFWEVNLKEKFSADLDQFPLGRKFLLQSGLKAKPRLKRSAPTTRAPSTKRKKVKK

SEQ ID NO: 40 - protein, 494 aa, synthetic construct
MPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKTAPPKEKEDPLNKYTFWEVNLKEKFSADLDQFPLGRKFLLQSGLKAKPRLKRSAPTTRAPSTKRKKVKK

SEQ ID NO: 41 - protein, 499 aa, synthetic construct
MPSDSTVYVPPPNPVSKVVATDAYVKRTNIFYHASSSRLLAVGHPYYSIKKVNKTVVPKVSGYQYRVFKVVLPDPNKFALPDSSLFDPTTQRLVWACTGLEVGRGQPLGVGVSGHPLLNKYDDVENSGGYGGNPGQDNRVNVGMDYKQTQLCMVGCAPPLGEHWGKGTQCSNTSVQNGDCPPLELITSVIQDGDMVDTGFGAMNFADLQTNKSDVPLDICGTVCKYPDYLQMAADPYGDRLFFYLRKEQMFARHFFNRAGTVGEPVPDDLLVKGGNNRSSVASSIYVHTPSGSLVSSEAQLFNKPYWLQKAQGHNNGICWGNHLFVTVVDTTRSTNMTLCASVSKSATYTNSDYKEYMRHVEEFDLQFIFQLCSITLSAEVMAYIHTMNPSVLEDWNFGLSPPPNGTLEDTYRYVQSQAITCQKPTPEKEKQDPYKDMSFWEVNLKEKFSSELDQFPLGRKFLLQSGYRGRTSARTGIKRPAVSKPSTAPKRKRTKTKK

SEQ ID NO: 42 - DNA, 1497 bp, synthetic construct
  source 1..1497: mol_type = other DNA
atgccctccgactccaccgtttacgttccaccaccaaatccagtttccaaggttgttgctactgacgcctacgtcaccagaactaatatcttctaccacgcctcctcatccagattgcttgctgttggtcacccatacttctccatcaagagagccaacaagaccgtcgttccaaaggtttctggttaccagtacagagtgttcaaggtcgtcttgccagatccaaacaagttcgctttgccagactcttccttgttcgacccaactactcagagattggtttgggcttgtaccggtttggaagttggtagaggtcaaccacttggtgttggtgtttctggacacccattcttgaacaagtacgacgacgttgaaaactccggttctggtggtaacccaggtcaagacaacagagttaacgtcggtatggactacaagcagacccagttgtgtatggttggttgtgctccaccattgggtgaacattggggtaagggtaagcagtgtaccaacactccagttcaagctggtgattgtccacctttggagttgatcacttccgttattcaggacggtgacatggttgacactggtttcggtgctatgaacttcgctgacttgcagactaacaagtccgacgttcctatcgacatctgtggtactacctgtaagtacccagactacttgcaaatggctgctgatccatacggtgacagactgttcttcttcctgagaaaagaacagatgttcgccagacacttcttcaacagagctggtgaagttggtgaaccagttccagacaccttgatcatcaaaggttccggtaacagaacctccgttggttcctctatctacgttaacactccatccggttctttggtgtcctctgaagctcagttgttcaacaagccatactggttgcaaaaggcccagggtcacaacaacggtatctgttggggtaaccagttgttcgttactgttgtcgacactaccagatccaccaatatgactttgtgtgcctccgttactacctcctccacttacactaactccgactacaaagagtacatgagacacgtcgaagagtacgacctgcagttcattttccagctgtgttccatcactttgtccgctgaggttatggcctacatccacactatgaacccatccgttttggaggactggaacttcggtttgtctccaccacctaacggtactttggaggatacctacagatacgttcagtcccaggctatcacttgccaaaagccaactccagagaaagagaagccagatccttacaagaacctgtccttctgggaagtcaacctgaaagaaaagttctccagcgagctggatcagtacccattgggtagaaagttcttgctgcagtccggttacagaggtagatcctccattagaaccggtgttaagaggcctgctgtttctaaggcttctgctgctccaaagaggaagagagctaagaccaagagataa

SEQ ID NO: 43 - DNA, 1491 bp, synthetic construct
  source 1..1491: mol_type = other DNA
atggactccaccgtctacgtcccaccaccaaatccagtttctaaggttgttgctaccgacgcctacgtcaccagaactaatatcttctaccacgcctcctcatccagattgcttgctgttggtcacccatacttctccatcaagagagccaacaagaccgtcgttccaaaggtttctggttaccagtacagagtgttcaaggtcgtcttgccagatccaaacaagttcgctttgccagactcttccttgttcgacccaactactcagagattggtttgggcttgtaccggtttggaagttggtagaggtcaaccacttggtgttggtgtttctggacacccattcttgaacaagtacgacgacgttgaaaactccggttctggtggtaacccaggtcaagacaacagagttaacgtcggtatggactacaagcagacccagttgtgtatggttggttgtgctccaccattgggtgaacattggggtaagggtaagcagtgtaccaacactccagttcaagctggtgattgtccacctttggagttgatcacttccgttattcaggacggtgacatggttgacactggtttcggtgctatgaacttcgctgacttgcagactaacaagtccgacgttcctatcgacatctgtggtactacctgtaagtacccagactacttgcaaatggctgctgatccatacggtgacagactgttcttcttcctgagaaaagaacagatgttcgccagacacttcttcaacagagctggtgaagttggtgaaccagttccagacaccttgatcatcaaaggttccggtaacagaacctccgttggttcctctatctacgttaacactccatccggttctttggtgtcctctgaagctcagttgttcaacaagccatactggttgcaaaaggcccagggtcacaacaacggtatctgttggggtaaccagttgttcgttactgttgtcgacactaccagatccaccaatatgactttgtgtgcctccgttactacctcctccacttacactaactccgactacaaagagtacatgagacacgtcgaagagtacgacctgcagttcattttccagctgtgttccatcactttgtccgctgaggttatggcctacatccacactatgaacccatccgttttggaggactggaacttcggtttgtctccaccacctaacggtactttggaggatacctacagatacgttcagtcccaggctatcacttgccaaaagccaactccagagaaagagaagccagatccttacaagaacctgtccttctgggaagtcaacctgaaagaaaagttctccagcgagctggatcagtacccattgggtagaaagttcttgctgcagtccggttacagaggtagatcctccattagaaccggtgttaagaggcctgctgtttccaaagcttctgctgctccaaagaggaagagagctaagaccaagagataa

SEQ ID NO: 44 - DNA, 1506 bp, synthetic construct
  source 1..1506: mol_type = other DNA
atgccatctgaggctaccgtttacttgccaccagttccagtttccaaggttgtttccactgacgagtacgtcgccagaactaacatctactaccacgctggtacttccagattgctggctgttggtcatccatacttcccaatcaagaagccaaacaacaacaagatcctggtgccaaaggtttccggtctgcagtacagagttttcagaatccacttgccagatccaaacaagttcggtttcccagacacatccttctacaacccagacactcagagattggtttgggcctgtgttggtgttgaagttggtagaggtcaaccacttggtgttggtatttctggtcacccattgctgaacaagttggacgatactgaaaacgcttccgcttacgctgctaacgctggtgttgataacagagaatgcatctccatggactacaagcagacccagttgtgtttgatcggttgcaagccacctattggtgaacattggggtaagggttctccatgtaccaacgttgctgttaacccaggtgattgtccaccattggagctgatcaacactgttattcaggacggtgacatggtcgacactggttttggtgctatggacttcactaccttgcaggctaacaagtccgaagttccattggacatctgtacctccatctgcaagtacccagactacatcaagatggtgtctgagccatacggtgactccctgttcttctacttgagaagagaacagatgttcgtcaggcacctgttcaacagagctggtgctgttggtgaaaacgttccagacgacctgtacatcaaaggttctggttctactgctaacctggcctcctctaactactttccaactccatccggttccatggttacttccgacgctcagattttcaacaagccatactggttgcaaagagcccagggtcacaacaacggtatctgttggggtaaccagttgttcgttactgttgtcgacactaccagatccaccaacatgtctttgtgtgctgctatctccacttccgagactacctacaagaacactaacttcaaagaatacctgaggcacggtgaagagtacgacttgcagttcattttccagctgtgcaagatcactttgaccgccgacgttatgacttacatccactccatgaactccactatcttggaggactggaactttggtctgcaaccaccaccaggtggtactttggaagatacctacagattcgttacttcccaggctatcgcttgtcagaagcacactccaccagctccaaaagaggatccattgaagaagtacactttctgggaagtcaacctgaaagagaagttctccgctgacttggatcagttcccactgggtagaaagttcttgttgcaggctggtttgaaggccaagccaaagttcactttgggtaagagaaaggctactccaactacttcctccacctctactaccgctaagaggaagaagagaaagttgtaa

SEQ ID NO: 45 - DNA, 1491 bp, synthetic construct
  source 1..1491: mol_type = other DNA
atggtctacttgccaccagttccagtttccaaggttgtttccactgacgagtacgtcgccagaactaacatctactaccacgctggtacttccagattgctggctgttggtcatccatacttcccaatcaagaagccaaacaacaacaagatcctggtgccaaaggtttccggtctgcagtacagagttttcagaatccacttgccagatccaaacaagttcggtttcccagacacatccttctacaacccagacactcagagattggtttgggcctgtgttggtgttgaagttggtagaggtcaaccacttggtgttggtatttctggtcacccattgctgaacaagttggacgatactgaaaacgcttccgcttacgctgctaacgctggtgttgataacagagaatgcatctccatggactacaagcagacccagttgtgtttgatcggttgcaagccacctattggtgaacattggggtaagggttctccatgtaccaacgttgctgttaacccaggtgattgtccaccattggagctgatcaacactgttattcaggacggtgacatggtcgacactggttttggtgctatggacttcactaccttgcaggctaacaagtccgaagttccattggacatctgtacctccatctgcaagtacccagactacatcaagatggtgtctgagccatacggtgactccctgttcttctacttgagaagagaacagatgttcgtcaggcacctgttcaacagagctggtgctgttggtgaaaacgttccagacgacctgtacatcaaaggttctggttctactgctaacctggcctcctctaactactttccaactccatccggttccatggttacttccgacgctcagattttcaacaagccatactggttgcaaagagcccagggtcacaacaacggtatctgttggggtaaccagttgttcgttactgttgtcgacactaccagatccaccaacatgtctttgtgtgctgctatctccacttccgagactacctacaagaacactaacttcaaagaatacctgaggcacggtgaagagtacgacttgcagttcattttccagctgtgcaagatcactttgaccgccgacgttatgacttacatccactccatgaactccactatcttggaggactggaactttggtctgcaaccaccaccaggtggtactttggaagatacctacagattcgttacttcccaggctatcgcttgtcagaagcacactccaccagctccaaaagaggatccattgaagaagtacactttctgggaagtcaacctgaaagagaagttctccgctgacttggatcagttcccactgggtagaaagttcttgttgcaggctggtttgaaggccaagccaaagttcactttgggtaagagaaaggctactccaactacttcctccacctctactaccgctaagaggaagaagagaaagttgtaa

SEQ ID NO: 46 - DNA, 1476 bp, synthetic construct
  source 1..1476: mol_type = other DNA
atggttccagtctccaaggttgtttccaccgacgaatacgttgccaggaccaacatctactaccacgctggtacttctagattgctggctgttggtcatccatacttcccaatcaagaagccaaacaacaacaagatcctggtgccaaaggtttccggtctgcagtacagagttttcagaatccacttgccagatccaaacaagttcggtttcccagacacatccttctacaacccagacactcagagattggtttgggcctgtgttggtgttgaagttggtagaggtcaaccacttggtgttggtatttctggtcacccattgctgaacaagttggacgatactgaaaacgcttccgcttacgctgctaacgctggtgttgataacagagaatgcatctccatggactacaagcagacccagttgtgtttgatcggttgcaagccacctattggtgaacattggggtaagggttctccatgtaccaacgttgctgttaacccaggtgattgtccaccattggagctgatcaacactgttattcaggacggtgacatggtcgacactggttttggtgctatggacttcactaccttgcaggctaacaagtccgaagttccattggacatctgtacctccatctgcaagtacccagactacatcaagatggtgtctgagccatacggtgactccctgttcttctacttgagaagagaacagatgttcgtcaggcacctgttcaacagagctggtgctgttggtgaaaacgttccagacgacctgtacatcaaaggttctggttctactgctaacctggcctcctctaactactttccaactccatccggttccatggttacttccgacgctcagattttcaacaagccatactggttgcaaagagcccagggtcacaacaacggtatctgttggggtaaccagttgttcgttactgttgtcgacactaccagatccaccaacatgtctttgtgtgctgctatctccacttccgagactacctacaagaacactaacttcaaagaatacctgaggcacggtgaagagtacgacttgcagttcattttccagctgtgcaagatcactttgaccgccgacgttatgacttacatccactccatgaactccactatcttggaggattggaactttggtctgcaaccaccaccaggtggtactttggaagatacctacagattcgttacttcccaggctatcgcttgtcagaagcacactccaccagctccaaaagaggatccattgaagaagtacactttctgggaagtcaacctgaaagagaagttctccgctgacttggatcagttcccactgggtagaaagttcttgttgcaggctggtttgaaggccaagccaaagttcactttgggtaagagaaaggctactccaactacttcctccacctctactaccgctaagaggaagaagagaaagttgtaa

SEQ ID NO: 47 - DNA, 1461 bp, synthetic construct
  source 1..1461: mol_type = other DNA
atggttgtttccaccgacgagtacgttgccagaactaacatctactaccacgctggtacttccagattgctggctgttggtcatccatacttcccaatcaagaagccaaacaacaacaagatcctggtgccaaaggtttccggtctgcagtacagagttttcagaatccacttgccagatccaaacaagttcggtttcccagacacatccttctacaacccagacactcagagattggtttgggcctgtgttggtgttgaagttggtagaggtcaaccacttggtgttggtatttctggtcacccattgctgaacaagttggacgatactgaaaacgcttccgcttacgctgctaacgctggtgttgataacagagaatgcatctccatggactacaagcagacccagttgtgtttgatcggttgcaagccacctattggtgaacattggggtaagggttctccatgtaccaacgttgctgttaacccaggtgattgtccaccattggagctgatcaacactgttattcaggacggtgacatggtcgacactggttttggtgctatggacttcactaccttgcaggctaacaagtccgaagttccattggacatctgtacctccatctgcaagtacccagactacatcaagatggtgtctgagccatacggtgactccctgttcttctacttgagaagagaacagatgttcgtcaggcacctgttcaacagagctggtgctgttggtgaaaacgttccagacgacctgtacatcaaaggttctggttctactgctaacctggcctcctctaactactttccaactccatccggttccatggttacttccgacgctcagattttcaacaagccatactggttgcaaagagcccagggtcacaacaacggtatctgttggggtaaccagttgttcgttactgttgtcgacactaccagatccaccaacatgtctttgtgtgctgctatctccacttccgagactacctacaagaacactaacttcaaagaatacctgaggcacggtgaagagtacgacttgcagttcattttccagctgtgcaagatcactttgaccgccgacgttatgacttacatccactccatgaactccactatcttggaggactggaactttggtctgcaaccaccaccaggtggtactttggaagatacctacagattcgttacttcccaggctatcgcttgtcagaagcacactccaccagctccaaaagaggatccattgaagaagtacactttctgggaagtcaacctgaaagagaagttctccgctgacttggatcagttcccactgggtagaaagttcttgttgcaggctggtttgaaggccaagccaaagttcactttgggtaagagaaaggctactccaactacttcctccacctctactaccgctaagaggaagaagagaaagttgtaa

SEQ ID NO: 48 - DNA, 1512 bp, synthetic construct
  source 1..1512: mol_type = other DNA
atgccctccgacaacaccgtctacttgccaccaccatctgttgccagagttgttaacactgacgactacgttactcccacctccattttctaccacgctggttcttctagattgctgaccgttggtaacccatacttcagagttccagctggtggtggtaacaagcaggacattccaaaggtttccgcctaccagtacagagtgttcagagtccaattgccagatccaaacaagttcggtttgccagacacctccatctacaacccagagactcaaagattggtttgggcttgtgctggtgtcgagattggtagaggtcaaccacttggtgttggtttgtctggtcacccattctacaacaagttggacgacactgaatcttcccacgctgctacttctaacgtttccgaggatgtcagagacaacgtgtccgttgactacaagcagacccagttgtgtatcttgggttgtgctccagctattggtgaacattgggctaagggaactgcctgtaagtccagaccattgtctcaaggtgattgcccaccattggagttgaagaacactgttttggaggacggtgacatggttgacactggttacggtgctatggacttctctaccttgcaggacactaagtgtgaggtcccattggacatttgtcagtccatctgtaagtaccccgactacttgcaaatgtccgctgatccatacggtgactctatgttcttctgcctgagaagagagcagttgttcgctagacacttctggaacagagctggtactatgggtgacactgttccacagtccttgtacatcaaaggtactggtatgagagcttccccaggttcctgtgtttactctccatctccatccggttccatcgttacttccgattctcagctgttcaacaagccatactggttgcacaaggctcagggtcataacaacggtgtttgttggcacaaccagctgttcgttactgttgttgacactaccagatccaccaacttgactatctgtgcttccactcaatccccagttccaggtcaatacgacgctactaagttcaagcagtactccagacacgtcgaagagtacgacttgcagttcatcttccagttgtgcactatcactttgaccgccgacgttatgtcctacatccactctatgaactcctccatcttggaggactggaactttggtgttcctcctcctccaactacttccttggttgatacctacagattcgttcagtccgtcgctatcacttgtcaaaaggatgctgctcctgctgagaacaaggacccatacgataagctgaagttctggaacgtcgacctgaaagagaagttctccttggacttggatcagtacccactgggtagaaagttcttggtccaagccggtttgagaagaaagccaactatcggtccaagaaagagatctgctccttccgctactacttcttccaagccagctaagagagttagagtcagagccagaaagtaa

SEQ ID NO: 49 - DNA, 1506 bp, synthetic construct
  source 1..1506: mol_type = other DNA
atggacaacaccgtctacctgccaccaccatctgttgctagagttgttaacaccgacgactacgttactcccacctccattttctaccacgctggttcttctagattgctgaccgttggtaacccatacttcagagttccagctggtggtggtaacaagcaggacattccaaaggtttccgcctaccagtacagagtgttcagagtccaattgccagatccaaacaagttcggtttgccagacacctccatctacaacccagagactcaaagattggtttgggcttgtgctggtgtcgagattggtagaggtcaaccacttggtgttggtttgtctggtcacccattctacaacaagttggacgacactgaatcttcccacgctgctacttctaacgtttccgaggatgtcagagacaacgtgtccgttgactacaagcagacccagttgtgtatcttgggttgtgctccagctattggtgaacattgggctaagggaactgcctgtaagtccagaccattgtctcaaggtgattgcccaccattggagttgaagaacactgttttggaggacggtgacatggttgacactggttacggtgctatggacttctctaccttgcaggacactaagtgtgaggtcccattggacatttgtcagtccatctgtaagtaccccgactacttgcaaatgtccgctgatccatacggtgactctatgttcttctgcctgagaagagagcagttgttcgctagacacttctggaacagagctggtactatgggtgacactgttccacagtccttgtacatcaaaggtactggtatgagagcttccccaggttcctgtgtttactctccatctccatccggttccatcgttacttccgattctcagctgttcaacaagccatactggttgcacaaggctcagggtcataacaacggtgtttgttggcacaaccagctgttcgttactgttgttgacactaccagatccaccaacttgactatctgtgcttccactcaatccccagttccaggtcaatacgacgctactaagttcaagcagtactccagacacgtcgaagagtacgacttgcagttcatcttccagttgtgcactatcactttgaccgccgacgttatgtcctacatccactctatgaactcctccatcttggaggactggaactttggtgttcctcctcctccaactacttccttggttgatacctacagattcgttcagtccgtcgctatcacttgtcaaaaggatgctgctcctgctgagaacaaggacccatacgataagctgaagttctggaacgtcgacctgaaagagaagttctccttggacttggatcagtacccactgggtagaaagttcttggtccaagccggtttgagaagaaagccaactatcggtccaagaaagagatctgctccttccgctactacttcttccaagccagctaagagagttagagtcagagccagaaagtaa

SEQ ID NO: 50 - DNA, 1503 bp, synthetic construct
  source 1..1503: mol_type = other DNA
atgccatctgaggctaccgtttacttgccaccagttccagtttccaaggttgtttccactgacgagtacgtcaccaggaccaacatctactaccatgctggttctgctagattgctgactgttggtcacccttactactccattccaaagtctgacaaccccaagaagatcgtcgttccaaaggtttccggactgcagtacagagtgttcagagtcagattgccagatccaaacaagttcggtttccccgacacatccttctacaacccagagactcaaagattggtttgggcctgtgttggtttggaggttggtagaggtcaaccacttggtgttggtatttccggtcatccactgctgaacaagtttgacgacactgagaactccaacagatacgctggtggtccaggtactgacaacagagaatgtatctccatggactacaagcagacccagttgtgtttgttgggttgcaagccacctattggtgaacattggggtaagggttctccatgttccaacaacgctattaccccaggtgattgtccaccattggagttgaagaactccgttattcaggacggtgacatggttgacactggtttcggtgctatggatttcactgccttgcaagacaccaagtccaacgttccattggacatctgtaactccatctgcaagtacccagactacttgaagatggttgctgagccatacggtgacaccttgttcttctacctgagaagagaacagatgttcgtcagacacttcttcaacagatccggtactgttggtgagtccgttccaactgacctgtacatcaaaggttctggttccactgctactttggccaactctacttacttcccaactccatccggttccatggttacttccgacgctcagattttcaacaagccatactggatgcaaagagcccagggtcacaacaacggtatttgttggggtaaccagttgttcgttaccgttgtcgacactaccagatccaccaacatgtctgtttgtgctgctatcgctaactccgacaccactttcaagtcctccaacttcaaagaatacctgagacacggtgaagagttcgacctgcagttcattttccagctgtgcaagatcactttgtccgccgacatcatgacctacatccactctatgaacccagccatcttggaggattggaacttcggtttgactactccaccatctggatccttggaggacacctacagattcgttacttctcaggccatcacctgtcaaaagactgctccacaaaagccaaaagaggacccattcaaggactacgtgttctgggaagtcaacttgaaagagaagttctccgctgacctggatcagttcccattgggtagaaagttcttgttgcaggctggttacagagccagaccaaagtttaaggctggtaagagatctgctccttccgcttctactactaccccagccaagagaaagaaaactaagaagtaa

SEQ ID NO: 51 - DNA, 1488 bp, synthetic construct
  source 1..1488: mol_type = other DNA
atgccatctgaggctaccgtttacttgccaccagttccagtttccaaggttgtttccactgacgagtacgtttccagaacctccatctactactacgctggttcttccagattgctggctgttggtcacccatacttctccatcaagaacccaactaacgccaagaagttgctggtcccaaaggtttctggattgcagtacagagtgttcagagtcagattgccagatccaaacaagttcggtttccccgacacatccttctacaacccagacactcaaagattggtttgggcctgtgttggtttggagattggtagaggtcaaccacttggtgttggtatctctggtcaccctttgctgaacaagtttgacgacactgagactggtaacaagtacccaggtcaacctggtgctgacaacagagaatgtctgtctatggactacaagcagacccagttgtgtttgttgggttgtaagccaccaactggtgaacattggggtaagggtgttgcttgtactaatgctgctccagctaacgactgtccaccattggagttgattaacaccatcatcgaggacggtgacatggttgatactggtttcggttgcatggacttcaagaccttgcaagctaacaagtccgacgtgccaattgacatctgtggttccacttgtaagtaccccgactacttgaagatgacctctgagccatacggtgactccctgttctttttcttgcgtcgtgaacagatgttcgtcagacacttcttcaacagagccggtactttgggtgaagctgttccagacgacctgtacatcaaaggttctggtactaccgcttccattcagtcctctgctttcttcccaactccatccggttctatggttacttctgagtcccagctgttcaacaagccatactggttgcaaagagcccagggtcacaacaacggtatctgttggggtaaccaggttttcgttactgttgtcgacactaccagatccaccaacatgactttgtgtactcaggttacttccgactccacctacaagaacgagaacttcaaagagtacatcaggcacgtcgaagagtacgacttgcagttcgttttccagttgtgcaaggtcactttgaccgctgaggttatgacttacatccacgctatgaaccccgacattttggaggattggcaattcggtttgactccaccaccatctgcttctctgcaagacacctacagattcgttacttcccaggccatcacctgtcaaaagaccgttccaccaaaagagaaagaggacccattgggcaagtacactttctgggaagttgacctgaaagagaagttctccgctgacttggatcagttcccactgggtagaaagttcttgttgcaggctggtttgaaggccaagccaaagttgaaaagagctgctccaacctccactagaacctcttccgctaaaagaaagaaggtcaagaagtaa

SEQ ID NO: 52 - DNA, 1497 bp, synthetic construct
  source 1..1497: mol_type = other DNA
atgtccaacgaggctaccgtttacttgccaccagtttccgtttccaaggttgtttccactgacgagtacgtcaccaggaccaacatctactaccatgctggttcttccagattgctggctgttggtcatccttactacgccattaagaagcaggactccaacaagatcgccgttccaaaggtttctggtctgcagtacagagtgttcagagtcaagttgccagatccaaacaagttcggtttccccgacacttctttctacgaccctgcttctcaaagattggtttgggcttgtaccggtgttgaggttggtagaggtcaaccacttggtgttggtatttctggtcacccattgctgaacaagttggacgacactgagaactctaacaagtacgtcggtaactccggtactgacaacagagaatgtatctccatggactacaagcagacccagttgtgtttgatcggttgcagaccacctattggtgaacattggggtaagggtactccatgtaacgccaaccaggttaaggctggtgaatgtccaccattggagttgttgaacactgtcttgcaggacggtgacatggttgatactggtttcggtgctatggacttcactaccttgcaagctaacaagtccgacgttccattggacatctgttcctccatctgtaagtacccagactacctgaagatggtgtctgaaccatacggcgacatgttgttcttctacctgagaagagaacaaatgttcgtcaggcacctgttcaacagagctggtactgttggtgaaactgttccagccgacttgtacatcaagggtactactggtactttgccctccacctcttacttcccaactccatctggttccatggttacttccgacgctcagatcttcaacaagccatactggttgcaaagagcccagggtcacaacaacggtatctgttggtctaaccagttgttcgttaccgttgtcgacactaccagatccaccaacatgtctgtttgttccgctgtttctacctccgactccacttacaagaacgacaacttcaaagaatacctgagacacggtgaagagtacgacctgcagttcattttccagctgtgcaagatcactttgaccgccgacgttatgacctacatccactctatgaacccatccatcttggaggactggaacttcggtttgactccaccaccatctggaactttggaggacacctacagatacgtcacttcccaggctgttacttgtcaaaagccatctgctccaaagccaaaggacgacccattgaagaactacactttctgggaagtcgacctgaaagaaaagttctccgctgacttggatcagttcccactgggtagaaagttcttgttgcaggctggtttgaaggccagaccaaacttcagattgggtaagagagctgctccagcttccacttctaagaagtcctccaccaagagaagaaaggtcaagtcttaa

SEQ ID NO: 53 - DNA, 1593 bp, synthetic construct
  source 1..1593: mol_type = other DNA
atgattatcatcttcctgaagaacgtcaacgtgttcccaatcttcttgcagatggctttgtggcgtccatctgactccactgtttatttgccaccaccatccgttgccagagttgtttctactgacgactacgtttccaggacctccattttctaccacgctggttcttccagattgctgactgttggtaacccatacttcagagtcgttccaaacggtgctggtaacaagcaagctgttccaaaggtttccgcctaccagtacagagttttcagagttgctttgccagatccaaacaagttcggtttgccagactctaccatctacaacccagagactcagagattggtttgggcctgtgttggtatggaaatcggtagaggtcagccacttggtattggtttgtctggtcacccattctacaacaagttggacgacactgaatctgctcacgctgctactgctgttatcactcaagacgtcagagacaacgtttccgtcgactacaagcagacccagttgtgtatcttgggttgtgttccagctattggtgagcattgggctaagggtactttgtgtaagccagctcaattgcagccaggtgattgtccaccattggagttgaagaacaccatcattgaggacggtgacatggttgacactggttacggtgctatggacttctctaccttgcaggacactaagtgtgaggtcccattggacatttgtcagtccatctgtaagtaccccgactacttgcaaatgtccgctgatccatacggtgactctatgttcttctgcctgagaagagagcagttgttcgctagacacttctggaacagagctggtgttatgggtgacactgttccaaccgacctgtacattaagggaacttccgctaacatgagagaaaccccaggttcctgtgtttactctccatctccatccggttccatcatcacttccgattctcagctgttcaacaagccatactggttgcacaaggctcagggtcacaacaacggtatctgttggcacaaccagctgttcgttactgttgttgacactaccagatccaccaacttgactttgtgtgcctccactcaaaacccagttccatctacttacgaccccaccaagttcaagcaatactccagacacgttgaagagtacgacctgcagttcatcttccagttgtgcactatcactttgaccgccgaggttatgtcctacatccactctatgaactcctccatcttggagaactggaacttcggtgttcctcctcctccaactacttccttggttgatacctacagattcgttcagtccgttgccgttacttgtcagaaggacactactccaccagagaagcaagacccatacgacaagttgaagttctggaccgtcgacctgaaagagaagttctcctctgacttggatcaatacccactgggtagaaagttcttggtccaggctggtttgagaagaaggccaactatcggtccaagaaaaagaccagctgcttctacttctactgcttccactgcttctagaccagccaagagagttagaatcagatccaagaagtaa

SEQ ID NO: 54 - DNA, 1572 bp, synthetic construct
  source 1..1572: mol_type = other DNA
atggtcaacgtgttcccaatcttcttgcagatggctttgtggcgtccatctgactccactgtttatttgccaccaccatccgttgccagagttgtttctactgacgactacgtttccaggacctccattttctaccacgctggttcttccagattgctgactgttggtaacccatacttcagagtcgttccaaacggtgctggtaacaagcaagctgttccaaaggtttccgcctaccagtacagagttttcagagttgctttgccagatccaaacaagttcggtttgccagactctaccatctacaacccagagactcagagattggtttgggcctgtgttggtatggaaatcggtagaggtcagccacttggtattggtttgtctggtcacccattctacaacaagttggacgacactgaatctgctcacgctgctactgctgttatcactcaagacgtcagagacaacgtttccgtcgactacaagcagacccagttgtgtatcttgggttgtgttccagctattggtgagcattgggctaagggtactttgtgtaagccagctcaattgcagccaggtgattgtccaccattggagttgaagaacaccatcattgaggacggtgacatggttgacactggttacggtgctatggacttctctaccttgcaggacactaagtgtgaggtcccattggacatttgtcagtccatctgtaagtaccccgactacttgcaaatgtccgctgatccatacggtgactctatgttcttctgcctgagaagagagcagttgttcgctagacacttctggaacagagctggtgttatgggtgacactgttccaaccgacctgtacattaagggaacttccgctaacatgagagaaaccccaggttcctgtgtttactctccatctccatccggttccatcatcacttccgattctcagctgttcaacaagccatactggttgcacaaggctcagggtcacaacaacggtatctgttggcacaaccagctgttcgttactgttgttgacactaccagatccaccaacttgactttgtgtgcctccactcaaaacccagttccatctacttacgaccccaccaagttcaagcaatactccagacacgttgaagagtacgacctgcagttcatcttccagttgtgcactatcactttgaccgccgaggttatgtcctacatccactctatgaactcctccatcttggagaactggaacttcggtgttcctcctcctccaactacttccttggttgatacctacagattcgttcagtccgttgccgttacttgtcagaaggacactactccaccagagaagcaagacccatacgacaagttgaagttctggaccgtcgacctgaaagagaagttctcctctgacttggatcaatacccactgggtagaaagttcttggtccaggctggtttgagaagaaggccaactatcggtccaagaaaaagaccagctgcttctacttctactgcttccactgcttctagaccagccaagagagttagaatcagatccaagaagtaa

SEQ ID NO: 55 - DNA, 1557 bp, synthetic construct
  source 1..1557: mol_type = other DNA
atgatcttcctgcagatggctttgtggcgtccatctgactctactgtttacttgccaccaccatccgttgccagagttgtttctactgacgactacgtttccaggacctccattttctaccacgctggttcttccagattgctgactgttggtaacccatacttcagagtcgttccaaacggtgctggtaacaagcaagctgttccaaaggtttccgcctaccagtacagagttttcagagttgctttgccagatccaaacaagttcggtttgcctgactccaccatctacaacccagagactcaaagattggtttgggcctgtgtcggtatggaaattggtagaggtcagccacttggtatcggtttgtctggtcacccattctacaacaagttggacgacactgaatctgctcacgctgctactgctgttatcactcaagacgtcagagacaacgtttccgtcgactacaagcagacccagttgtgtatcttgggttgtgttccagctattggtgagcattgggctaagggtactttgtgtaagccagctcaattgcagccaggtgattgtccaccattggagttgaagaacaccatcattgaggacggtgacatggttgacactggttacggtgctatggacttctctaccttgcaggacactaagtgtgaggtcccattggacatttgtcagtccatctgtaagtaccccgactacttgcaaatgtccgctgatccatacggtgactctatgttcttctgcctgagaagagagcagttgttcgctagacacttctggaacagagctggtgttatgggtgacactgttccaaccgacctgtacattaagggaacttccgctaacatgagagaaaccccaggttcctgtgtttactctccatctccatccggttccatcatcacttccgattctcagctgttcaacaagccatactggttgcacaaggctcagggtcacaacaacggtatctgttggcacaaccagctgttcgttactgttgttgacactaccagatccaccaacttgactttgtgtgcctccactcaaaacccagttccatctacttacgaccccaccaagttcaagcaatactccagacacgttgaagagtacgacctgcagttcatcttccagttgtgcactatcactttgaccgccgaggttatgtcctacatccactctatgaactcctccatcttggagaactggaacttcggtgttcctcctcctccaactacttccttggttgatacctacagattcgttcagtccgttgccgttacttgtcagaaggacactactccaccagagaagcaagacccatacgacaagttgaagttctggaccgtcgacctgaaagagaagttctcctctgacttggatcaatacccactgggtagaaagttcttggtccaggctggtttgagaagaaggccaactatcggtccaagaaaaagaccagctgcttctacttctactgcttccactgcttctagaccagccaagagagttagaatcagatccaagaagtaa

SEQ ID NO: 56 - DNA, 1530 bp, synthetic construct
  source 1..1530: mol_type = other DNA
atgccatctgactccaccgtttatttgccaccaccatccgttgctagagttgtttctactgacgactacgtgtccagaacctccattttctaccacgctggttcttccagattgctgactgttggtaacccatacttcagagtcgttccaaacggtgctggtaacaagcaagctgttccaaaggtttccgcctaccagtacagagttttcagagttgctttgccagatccaaacaagttcggtttgccagactctaccatctacaacccagagactcagagattggtttgggcctgtgttggtatggaaatcggtagaggtcagccacttggtattggtttgtctggtcacccattctacaacaagttggacgacactgaatctgctcacgctgctactgctgttatcactcaagacgtcagagacaacgtttccgtcgactacaagcagacccagttgtgtatcttgggttgtgttccagctattggtgagcattgggctaagggtactttgtgtaagccagctcaattgcagccaggtgattgtccaccattggagttgaagaacaccatcattgaggacggtgacatggttgacactggttacggtgctatggacttctctaccttgcaggacactaagtgtgaggtcccattggacatttgtcagtccatctgtaagtaccccgactacttgcaaatgtccgctgatccatacggtgactctatgttcttctgcctgagaagagagcagttgttcgctagacacttctggaacagagctggtgttatgggtgacactgttccaaccgacctgtacattaagggaacttccgctaacatgagagaaaccccaggttcctgtgtttactctccatctccatccggttccatcatcacttccgattctcagctgttcaacaagccatactggttgcacaaggctcagggtcacaacaacggtatctgttggcacaaccagctgttcgttactgttgttgacactaccagatccaccaacttgactttgtgtgcctccactcaaaacccagttccatctacttacgaccccaccaagttcaagcaatactccagacacgttgaagagtacgacctgcagttcatcttccagttgtgcactatcactttgaccgccgaggttatgtcctacatccactctatgaactcctccatcttggagaactggaacttcggtgttcctcctcctccaactacttccttggttgatacctacagattcgttcagtccgttgccgttacttgtcagaaggacactactccaccagagaagcaagacccatacgacaagttgaagttctggaccgtcgacctgaaagagaagttctcctctgacttggatcaatacccactgggtagaaagttcttggtccaggctggtttgagaagaaggccaactatcggtccaagaaaaagaccagctgcttctacttctactgcttccactgcttctagaccagccaagagagttagaatcagatccaagaagtaa

SEQ ID NO: 57 - DNA, 1500 bp, synthetic construct
  source 1..1500: mol_type = other DNA
atgccatctgaggctaccgtttacttgccaccagttccagtttccaaggttgtttccactgacgagtacgtttccagaacctccatctactactacgctggttcttccagattgctgactgttggtcacccatacttctctatcaagaacacctcttccggtaacggtaagaaggtcttggtcccaaaggtttccggattgcagtacagagtgttcagaatcaagctgccagatccaaacaagttcggtttcccagacacatccttctacaacccagagactcagagattggtttgggcttgtactggtttggagatcggtagaggtcaaccacttggtgttggtatttctggtcaccctctgctgaacaagtttgacgacactgagacttccaacaagtacgctggtaagccaggtatcgacaacagagaatgcctgtctatggactacaagcagacccagttgtgtatcttgggttgcaagccacctattggtgaacattggggtaagggtactccatgcaacaacaactctggtaaccctggtgattgtccaccattgcagttgattaactccgtcattcaggacggtgacatggttgatactggtttcggttgtatggacttcaacaccttgcaagcttccaagtccgacgtgccaattgacatttgttcctccgtctgtaagtaccccgactacttgcaaatggcttctgagccatacggtgactccctgttctttttcttgcgtcgtgaacagatgttcgtcagacacttcttcaacagagccggtactttgggtgatccagttcctggtgacttgtacattcaaggttccaactccggtaacaccgctactgttcaatcttccgctttcttcccaactccatccggttctatggttacttctgagtcccagctgttcaacaagccatactggttgcaaagagcccagggtcacaacaacggtatctgttggggtaaccagttgttcgttactgtcgttgacactaccagatccaccaacatgactttgtgtgccgaggtcaagaaagagtccacctacaagaacgagaacttcaaagaatacctgagacacggtgaagagttcgacctgcagttcattttccagctgtgcaagatcactttgaccgccgacgttatgacctacatccacaagatggacgctaccattttggaggactggcaattcggtttgactcctcctccttctgcttccttggaagatacctacagattcgttacttccaccgccatcacctgtcagaagaacactccaccaaagggtaaagaggacccactgaaggattacatgttctgggaagtcgacctgaaagagaagttctctgctgacttggatcagttcccactgggtagaaagttcttgttgcaagctggtctgcaggccagaccaaagttgaaaagacctgcttcttccgctccaagaacttccaccaagaagaagaaggttaagagataa

SEQ ID NO: 58 - DNA, 1548 bp, synthetic construct
  source 1..1548: mol_type = other DNA
atgatcttgttctgtgtcgccgacgtcaacgtgttccacattttcttgcaaatgtccgtctggcgtccatccgaagctactgtttatttgccaccagttccagtctccaaggttgtttctactgacgagtacgtttccaggacctccatctactactacgctggttcttctagattgctggccgttggtaacccatacttctccattaagtccccaaacaacaacaagaaggtgctggtcccaaaggtttccggattgcagtacagagtgttcagagtcagattgccagatccaaacaagttcggtttccccgacacatccttctacaacccagacactcaaagattggtttgggcctgtgttggtttggagattggtagaggtcaaccacttggtgttggtgtttctggtcacccatacctgaacaagtttgacgacactgagacttccaacagatacccagctcaaccaggttccgacaacagagaatgtctgtctatggactacaagcagacccagttgtgtctgatcggttgtaagccaccaactggtgaacattggggtaagggtgttgcctgtaacaacaacgctgctgctactgattgtccaccattggagctgttcaactccattattgaggacggtgacatggtcgacactggtttcggttgtatggatttcggtactctgcaggctaacaagtccgacgttccaatcgacatctgtaactccacctgtaagtacccagactacttgaagatggcttccgagccatacggtgattccttgttctttttcctgcgtcgtgaacagatgttcgtcagacacttcttcaacagagccggtaagttgggtgaagctgttccagacgacctgtacatcaaaggttctggtaacaccgctgttattcagtcctctgctttcttcccaactccatccggttctatcgttacttctgagtcccagttgttcaacaagccatactggttgcaaagagcccagggtcacaacaacggtatttgttggggtaaccagctgttcgttactgttgttgacactaccagatccaccaacatgaccttgtgtaccgaggttaccaaagagggaacctacaagaacgacaacttcaaagaatacgtcaggcacgtcgaagagtacgacttgcaattcgttttccagctgtgcaagatcactttgaccgctgagatcatgacctacatccacactatggactccaacatcttggaggactggcaattcggtttgactccaccaccatctgcttctctgcaagacacctacagattcgtcacttcccaggctatcacctgtcaaaagactgctccaccaaaagagaaagaggacccattgaacaagtacactttctgggaagtcaacctgaaagagaagttctccgctgacttggatcagttcccactgggtagaaagttcttgttgcagtccggtttgaaggccaagccaagattgaagagatccgctccaactactagagccccatccactaagaggaagaaggttaagaagtaa

SEQ ID NO: 59 - DNA, 1527 bp, synthetic construct
  source 1..1527: mol_type = other DNA
atggtcaacgtgttccacatcttcttgcagatgtctgtttggaggccatccgaggctactgtttacttgccaccagttccagtttccaaggttgtttccactgacgagtacgtttccagaacctccatctactactacgctggttcttccagattgctggctgttggtaacccatacttctccatcaagtccccaaacaacaacaagaaggtgctggtcccaaaggtttccggattgcagtacagagtgttcagagtcagattgccagatccaaacaagttcggtttccccgacacatccttctacaacccagacactcaaagattggtttgggcctgtgttggtttggagattggtagaggtcaaccacttggtgttggtgtttctggtcacccatacctgaacaagtttgacgacactgagacttccaacagatacccagctcaaccaggttccgacaacagagaatgtctgtctatggactacaagcagacccagttgtgtctgatcggttgtaagccaccaactggtgaacattggggtaagggtgttgcctgtaacaacaacgctgctgctactgattgtccaccattggagctgttcaactccattattgaggacggtgacatggtcgacactggtttcggttgtatggatttcggtactctgcaggctaacaagtccgacgttccaatcgacatctgtaactccacctgtaagtacccagactacctgaagatggcttctgagccatacggtgattccctgttctttttcttgcgtcgtgaacagatgttcgtcagacacttcttcaacagagccggtaagttgggtgaagctgttccagacgacctgtacatcaaaggttctggtaacaccgctgttattcagtcctctgctttcttcccaactccatccggttctatcgttacttctgagtcccagttgttcaacaagccatactggttgcaaagagcccagggtcacaacaacggtatttgttggggtaaccagctgttcgttactgttgttgacactaccagatccaccaacatgaccttgtgtaccgaggttaccaaagagggaacctacaagaacgacaacttcaaagaatacgtcaggcacgtcgaagagtacgacttgcaattcgttttccagctgtgcaagatcactttgaccgctgagatcatgacctacatccacactatggactccaacatcttggaggactggcaattcggtttgactccaccaccatctgcttctctgcaagacacctacagattcgtcacttcccaggctatcacctgtcaaaagactgctccaccaaaagagaaagaggacccattgaacaagtacactttctgggaagtcaacctgaaagagaagttctccgctgacttggatcagttcccactgggtagaaagttcttgttgcagtccggtttgaaggccaagccaagattgaagagatccgctccaactactagagccccatccactaagaggaagaaggttaagaagtaa

SEQ ID NO: 60 - DNA, 1512 bp, synthetic construct
  source 1..1512: mol_type = other DNA
atgatcttcctgcagatgtctgtttggaggccatctgaggctactgtttacttgccaccagttccagtttccaaggttgtttccactgacgagtacgtttccagaacctccatctactactacgctggttcttccagattgctggctgttggtaacccatacttctccatcaagtccccaaacaacaacaagaaggtgctggtcccaaaggtttccggattgcagtacagagtgttcagagtcagattgccagatccaaacaagttcggtttccccgacacatccttctacaacccagacactcaaagattggtttgggcctgtgttggtttggagattggtagaggtcaaccacttggtgttggtgtttctggtcacccatacctgaacaagtttgacgacactgagacttccaacagatacccagctcaaccaggttccgacaacagagaatgtctgtctatggactacaagcagacccagttgtgtctgatcggttgtaagccaccaactggtgaacattggggtaagggtgttgcctgtaacaacaacgctgctgctactgattgtccaccattggagctgttcaactccattattgaggacggtgacatggtcgacactggtttcggttgtatggatttcggtactctgcaggctaacaagtccgacgttccaatcgacatctgtaactccacctgtaagtacccagactacctgaagatggcttctgagccatacggtgattccctgttctttttcttgcgtcgtgaacagatgttcgtcagacacttcttcaacagagccggtaagttgggtgaagctgttccagacgacctgtacatcaaaggttctggtaacaccgctgttattcagtcctctgctttcttcccaactccatccggttctatcgttacttctgagtcccagttgttcaacaagccatactggttgcaaagagcccagggtcacaacaacggtatttgttggggtaaccagctgttcgttactgttgttgacactaccagatccaccaacatgaccttgtgtaccgaggttaccaaagagggaacctacaagaacgacaacttcaaagaatacgtcaggcacgtcgaagagtacgacttgcaattcgttttccagctgtgcaagatcactttgaccgctgagatcatgacctacatccacactatggactccaacatcttggaggactggcaattcggtttgactccaccaccatctgcttctctgcaagacacctacagattcgtcacttcccaggctatcacctgtcaaaagactgctccaccaaaagagaaagaggacccattgaacaagtacactttctgggaagtcaacctgaaagagaagttctccgctgacttggatcagttcccactgggtagaaagttcttgttgcagtccggtttgaaggccaagccaagattgaagagatccgctccaactactagagccccatccactaagaggaagaaggttaagaagtaa

SEQ ID NO: 61 - DNA, 1485 bp, synthetic construct
  source 1..1485: mol_type = other DNA
atgccatctgaggctaccgtttacttgccaccagttccagtttccaaggttgtttccactgacgagtacgtttccagaacctccatctactactacgctggttcttccagattgctggctgttggtaacccatacttctccatcaagtccccaaacaacaacaagaaggtgctggtcccaaaggtttccggattgcagtacagagtgttcagagtcagattgccagatccaaacaagttcggtttccccgacacatccttctacaacccagacactcaaagattggtttgggcctgtgttggtttggagattggtagaggtcaaccacttggtgttggtgtttctggtcacccatacctgaacaagtttgacgacactgagacttccaacagatacccagctcaaccaggttccgacaacagagaatgtctgtctatggactacaagcagacccagttgtgtctgatcggttgtaagccaccaactggtgaacattggggtaagggtgttgcctgtaacaacaacgctgctgctactgattgtccaccattggagctgttcaactccattattgaggacggtgacatggtcgacactggtttcggttgtatggatttcggtactctgcaggctaacaagtccgacgttccaatcgacatctgtaactccacctgtaagtacccagactacttgaagatggcttccgagccatacggtgattccttgttctttttcctgcgtcgtgaacagatgttcgtcagacacttcttcaacagagccggtaagttgggtgaagctgttccagacgacctgtacatcaaaggttctggtaacaccgctgttattcagtcctctgctttcttcccaactccatccggttctatcgttacttctgagtcccagttgttcaacaagccatactggttgcaaagagcccagggtcacaacaacggtatttgttggggtaaccagctgttcgttactgttgttgacactaccagatccaccaacatgaccttgtgtaccgaggttaccaaagagggaacctacaagaacgacaacttcaaagaatacgtcaggcacgtcgaagagtacgacttgcaattcgttttccagctgtgcaagatcactttgaccgctgagatcatgacctacatccacactatggactccaacatcttggaggactggcaattcggtttgactccaccaccatctgcttctctgcaagacacctacagattcgtcacttcccaggctatcacctgtcaaaagactgctccaccaaaagagaaagaggacccattgaacaagtacactttctgggaagtcaacctgaaagagaagttctccgctgacttggatcagttcccactgggtagaaagttcttgttgcagtccggtttgaaggccaagccaagattgaagagatccgctccaactactagagccccatccactaagaggaagaaggttaagaagtaa

SEQ ID NO: 62 - DNA, 1497 bp, synthetic construct
  source 1..1497: mol_type = other DNA
atgccatctgactccaccgtttatgtcccaccaccaaatccagtttccaaggttgttgctactgacgcctacgtcaagaggaccaatattttctaccacgcctcctcatccagattgctggctgttggtcatccttactactccatcaagaaggtcaacaagaccgtcgttccaaaggtttccggttaccagtacagagtgttcaaggtcgttttgccagatccaaacaagttcgctttgcctgactcttccttgttcgacccaactactcagagattggtttgggcttgtaccggtttggaagttggtagaggtcaaccacttggtgttggtgtttctggtcacccattgctgaacaagtacgacgacgttgaaaactccggtggttacggtggtaacccaggtcaagataacagagtcaacgtcggtatggactacaagcagacccagttgtgtatggttggttgtgctccaccattgggtgaacattggggtaagggtactcagtgttccaacacttccgttcagaacggtgattgtccacctttggagttgatcacctccgttattcaagacggtgacatggttgacactggtttcggtgctatgaacttcgctgacttgcagactaacaagtccgacgttcctttggacatctgtggtactgtctgtaagtacccagactacttgcaaatggctgctgatccatacggtgacagactgttcttctacctgagaaaagaacagatgttcgccagacacttcttcaacagagctggtactgttggtgaaccagttccagacgacttgttggtcaaaggtggtaacaacagatcctccgttgcttcctctatctacgttcacactccatccggttccttggtttcttctgaagcccagttgttcaacaagccatactggttgcaaaaggcccagggtcacaacaacggtatttgttggggtaaccacttgttcgttaccgttgtcgacactaccagatccaccaatatgactttgtgtgcctccgtttccaagtccgctacttacactaactccgactacaaagagtacatgagacacgtcgaagagttcgacctgcagttcattttccagctgtgttccattactttgtccgccgaggttatggcctacatccacactatgaacccatccgttttggaggactggaacttcggtttgtctccaccacctaacggtactttggaggatacctacagatacgttcagtcccaggctatcacttgccaaaagccaactccagagaaagagaagcaggacccatacaaggacatgtctttctgggaagtcaacctgaaagaaaagttctcctcagagctggatcagttcccactgggtagaaagttcttgttgcagtccggttacagaggtagaacttccgctagaactggtatcaagaggcctgctgtttctaagccatctactgccccaaagagaaagcgtaccaagactaagaag

SEQ ID NO: 63 - DNA, 1503 bp, synthetic construct
  source 1..1503: mol_type = other DNA
atgtggagaccttccgacagcacagtgtacgtgcccccccctaatccagtgagtaaggtggtcgccaccgatgcctacgtgacaagaaccaatatcttctatcacgcctcaagcagccgcctgctggctgttggccacccctacttctctattaaaagagccaacaagaccgtggtgcccaaggtgtccgggtaccagtaccgcgtgtttaaagtggtgctgcccgaccccaacaagttcgccctgcccgatagctccctgttcgacccgaccacccagaggctcgtgtgggcctgcaccggccttgaggtcgggcggggccagcctctgggggtgggagtcagtggccaccccttcctgaataagtatgacgatgtggagaactcagggagcggcggcaaccctggccaggacaacagagtgaacgtgggcatggactataagcagactcagctgtgtatggtgggctgcgccccacccctgggcgagcattggggcaagggcaagcagtgcaccaatacccccgtgcaggccggcgactgtcctcctctggagctgatcaccagcgtgatccaggacggggatatggtggataccggtttcggagccatgaatttcgccgacctgcagacgaacaagtccgatgtgcctatcgatatctgcggaaccacctgcaagtaccccgattacctgcagatggccgccgacccttacggcgatcgcctgttcttcttcctccggaaggagcagatgttcgcccgtcactttttcaatcgggccggcgaggtgggcgagcctgtgcccgatacactgatcatcaagggctccgggaaccggaccagcgtggggtccagcatctatgtgaatacaccctccggcagcctggtgtcttccgaggcccagctgttcaataagccatactggctccagaaagcccagggccacaataacggcatctgctggggcaaccagttgttcgtgacagtggtggatacaaccaggtccaccaacatgacactgtgcgccagcgtgaccacaagcagcacctacacaaactccgactataaagagtacatgcgccacgtggaggagtacgacctgcagttcattttccagctgtgtagcattaccctgagcgccgaagtgatggcctacattcacaccatgaaccccagcgtgctcgaagattggaacttcggcctctctccccccccaaatggcaccctggaagacacctatcggtacgtgcagagccaggcaattacatgtcagaagcccacccccgagaaggagaagcctgacccatacaagaacctgtcattctgggaggtgaatctgaaggagaagtttagcagcgagctggaccagtacccactgggtaggaaattcctgctgcagtccggctatcggggccgcagctccatccgcactggcgtgaagaggcccgccgtgtctaaggcctccgccgcccccaagcgcaaaagggccaagaccaagcgctga

SEQ ID NO: 64 - DNA, 1521 bp, synthetic construct
  source 1..1521: mol_type = other DNA
atgagcctgtggctgcccagcgaggccacagtttatctgcctccagtgcctgtgtccaaggtggtgtccaccgatgagtacgtggcccggaccaacatctactaccacgccggcacatctagactgctggccgtgggacacccttacttccccatcaagaagcccaacaacaacaagatcctggtgcctaaggtgtccggcctgcagtaccgggtgttcagaatccatctgcctgatcctaacaagttcggcttccccgacaccagcttctacaaccccgacacacagagactcgtgtgggcctgtgtgggcgtcgaagttggaagaggacagcctctcggcgtgggaatctctggacatcccctgctgaacaagctggacgacaccgagaatgcctctgcctatgccgctaatgctggcgtggacaaccgcgagtgcatcagcatggactacaagcagacccagctgtgcctgatcggctgcaagcctcctattggagagcactggggaaagggcagcccctgcacaaatgtggctgtgaaccctggcgattgccctcctctggaactgatcaacaccgtgatccaggacggcgacatggtggatacaggcttcggcgccatggacttcacaaccctgcaggccaacaagagcgaggtgcccctggatatctgcaccagcatctgcaagtaccccgactacatcaagatggtgtccgagccttacggcgacagcctgttcttttacctgcggcgcgagcagatgttcgtgcggcacctgtttaatagagccggcgctgtgggcgagaacgtgcccgatgatctgtacatcaaaggcagcggcagcaccgccaatctggccagcagcaactacttccctacacctagcggctccatggtcacaagcgacgcccagatcttcaacaagccctactggctgcagagagcccagggccacaacaatggcatctgctggggcaaccagctgttcgtgacagtggtggacacaacccggtccaccaacatgagtctgtgtgccgccatcagcaccagcgagacaacctacaagaacaccaacttcaaagagtacctgcgccacggcgaggaatacgacctgcagttcatctttcagctgtgcaagatcaccctgaccgccgacgtgatgacctacatccacagcatgaacagcaccatcctggaagattggaacttcggactgcagcctccacctggcggcactctggaagatacctacagattcgtgaccagccaggccattgcctgccagaagcacacacctccagctcctaaagaggaccctctgaagaagtacaccttctgggaagtgaacctgaaagagaagttcagcgccgacctggaccagtttcctctgggcagaaagttcctgctgcaagctggcctgaaggccaagcctaagttcaccctgggcaagagaaaggccacacctaccaccagcagcacaagcaccacagccaagcggaagaaacggaagctgtgataa

SEQ ID NO: 65 - DNA, 1512 bp, synthetic construct
  source 1..1512: mol_type = other DNA
atgccatccgacaatacagtgtacctgccccctccctcagtcgccagagtggtgaataccgacgattacgtgaccccaacctctatcttttaccacgccggcagctcaagactgctgacagtgggaaatccttactttagggtgccagcaggcggcggcaacaaacaggacatcccaaaagtgagcgcctatcagtaccgggtgtttagggtgcagctgcccgaccctaacaagttcggactgccagatacctctatctataatcccgagacccagcgcctggtgtgggcctgtgccggcgtggagatcggcagaggccagcccctgggagtgggcctgagcggccatcccttttacaacaaactcgatgacacagaaagctcacacgcagccacctccaacgtgagcgaggacgtaagggataacgtgagcgtggattataagcagacacagctgtgtatcctgggatgtgcccccgccatcggcgaacattgggccaaaggcaccgcctgcaaatccaggccactgagccagggcgactgtccacccctggagctgaaaaatactgtgctggaagatggagacatggtggataccggatacggggccatggacttttccacactgcaggacaccaagtgtgaagtgcccctggacatctgccagagcatctgtaagtacccagattacctgcagatgagcgccgacccctacggcgactctatgtttttttgtctgagacgggagcagctgttcgcccgccacttttggaatagagccggaactatgggcgacacagtgcctcagtctctgtacatcaagggcaccggcatgcgggcctccccaggctcttgtgtctactccccaagcccatccggcagcatcgtgacctctgacagccagctgttcaacaagccatactggctgcacaaggcccaggggcacaacaacggggtgtgctggcacaaccagctgttcgtgacagtggtggacaccactagatctaccaacctgaccatttgtgcctccacacagagtcccgtgcctggccagtatgacgccaccaagttcaaacagtactctagacacgtggaagagtacgacctgcagttcatcttccagctgtgcactatcaccctgactgcagacgtgatgtcttacattcacagcatgaacagctctatccttgaggattggaacttcggcgtccctcccccacctaccacaagcctggtggacacctacagattcgtgcagagcgtcgctattacatgtcagaaggatgccgcacccgccgagaacaaggacccatacgacaagctcaagttctggaacgtggatctgaaggagaaattcagtctggatctggaccagtatccactgggccgcaagtttctggtgcaggccgggctgcgcaggaagcctaccattggccctagaaagcggagcgctccatccgccactaccagtagcaagccagccaaaagggtgagagtgcgggcacggaaatga

SEQ ID NO: 66 - DNA, 1515 bp, synthetic construct
  source 1..1515: mol_type = other DNA
atgtctctgtggcggcctagcgaggctactgtctacttaccacctgtcccagtgtctaaagttgtaagcacggatgaatatgtaacacgaaccaacatatattatcacgcaggcagtgctaggctgcttacagtaggccatccatattattccatacctaaatctgacaatcctaaaaaaatagttgtaccaaaggtgtcaggattacaatatagggtatttagggttcgtttaccagatccaaacaaatttggatttcctgatacatctttttataatcctgaaactcaacgcttagtttgggcctgtgttggtttagaggtaggtcgcgggcagccattaggtgtaggtattagtggtcatccattattaaataaatttgatgacactgaaaactctaatagatatgccggtggtcctggcactgataatagggaatgtatatcaatggattataaacaaacacaactgtgtttacttggttgcaaaccacctattggagagcattggggtaaaggtagtccttgtagtaacaatgctattacccctggtgattgtcctccattagaattaaaaaattcagttatacaagatggggatatggttgatacaggctttggagctatggattttactgctttacaagacactaaaagtaatgttcctttggacatttgtaattctatttgtaaatatccagattatcttaaaatggttgctgagccatatggcgatacattatttttttatttacgtagggaacaaatgtttgtaaggcatttttttaatagatcaggcacggttggtgaatcggtccctactgacttatatattaaaggctccggttcaacagctactttagctaacagtacatactttcctacacctagcggctccatggttacttcagatgcacaaatttttaataaaccatattggatgcaacgtgctcagggacacaataatggtatttgttggggcaatcagttatttgttactgtggtagataccacacgtagtaccaatatgtctgtttgtgctgcaattgcaaacagtgatactacatttaaaagtagtaattttaaagagtatttaagacatggtgaggaatttgatttacaatttatatttcagttatgcaaaataacattatctgcagacataatgacatatattcacagtatgaatcctgctattttggaagattggaattttggattgaccacacctccctcaggttctttggaggatacctataggtttgtcacctcacaggccattacatgtcaaaaaactgccccccaaaagcccaaggaagatccatttaaagattatgtattttgggaggttaatttaaaagaaaagttttctgcagatttagatcagtttccactgggtcgcaaatttttattacaggcaggatatagggcacgtcctaaatttaaagcaggtaaacgtagtgcaccctcagcatctaccactacaccagcaaaacgtaaaaaaactaaaaagtaa

SEQ ID NO: 67 - DNA, 1488 bp, synthetic construct
  source 1..1488: mol_type = other DNA
atgccctccgaagccaccgtgtatctgccaccagtgccagtgagcaaggtggtgagcaccgacgagtacgtgagccggacctctatttactattacgccggctccagtaggctgctggccgtgggacacccctacttcagcatcaagaaccctacaaatgccaagaagctgctggttcccaaagtctccggtctgcagtacagagtgttcagggtgagactgcccgaccctaataagttcggctttccagatacatctttttacaatcctgacacacagagactggtgtgggcctgcgtgggcctggagattggacgggggcagcctctgggagtggggatcagcggacaccctctcctgaataagttcgatgatacagagaccggaaacaaatatccaggacagcctggcgccgataacagagagtgcctgagcatggactataagcagacccagctgtgtctgctgggctgcaagccacccaccggagagcactggggaaagggcgtggcctgcacaaatgccgccccagccaatgattgccccccactggagctgatcaacaccatcattgaggacggagacatggtggacaccggctttggatgtatggacttcaagaccctgcaggcaaataagtctgacgtgcctatcgacatttgtggcagcacctgcaaataccctgactacctgaagatgacttctgaaccttacggtgatagcctgttcttttttctgcggcgggaacagatgtttgtgagacacttttttaacagggctggcaccctgggggaggctgtgcctgatgacctgtacatcaagggcagcgggacaaccgctagcattcagtcctctgcctttttccccaccccctccggctccatggtgaccagcgagtcccagctgtttaacaagccatactggctgcagcgcgcccagggccacaataatggaatctgctggggaaaccaggtgttcgtgactgtggtggacaccacccgcagcacaaatatgaccctgtgtactcaggtgacctctgacagcacctacaagaatgagaactttaaggagtacatccggcacgtggaggagtacgacctccagtttgtgtttcagctgtgcaaagtgacactgaccgctgaggtgatgacttacatccacgccatgaatccagatatcctggaagactggcagttcggcctgacacctcccccatctgcaagcctgcaggatacctaccgcttcgtgacctctcaggccatcacatgccagaagacagtgccccctaaggagaaagaggacccactgggcaagtacactttctgggaggtggatctgaaggaaaagttttctgccgacctggatcagtttccactgggccgaaaattcctgctgcaggccgggctgaaggccaaacccaaactgaagagggcagcccccactagtacccgcacctcatccgccaaaagaaagaaagtgaagaagtga

SEQ ID NO: 68 - DNA, 1509 bp, synthetic construct
  source 1..1509: mol_type = other DNA
atgagtctgtggcggtctaacgaggccaccgtgtatctgcctcctgtgtctgtgagcaaggtggtgtctaccgacgaatatgtgacacgcaccaacatctactatcacgccggatctagcaggctgctggccgtgggacacccctactacgccatcaaaaagcaggatagcaacaagatcgccgttccaaaggtgagtgggctccagtaccgcgtcttcagagtgaagctgcctgatcccaacaagtttggatttcccgacaccagtttttacgaccccgcctcccagaggctggtctgggcctgcaccggagtggaggtcggccggggccagccactcggggtgggcatcagcgggcaccctctgctgaataagctggacgacacagagaattccaacaagtacgtgggcaactccggcactgacaatcgagaatgcatcagcatggactacaagcagacacagctgtgtctgatcggatgccggccaccaattggcgaacactggggcaagggcaccccctgcaacgccaatcaggtgaaggctggcgagtgcccacccctggagctgctgaatacagtgctgcaggatggcgacatggtggatactgggttcggcgctatggacttcaccacactgcaggccaacaagtccgatgtgcccctggacatctgcagcagcatctgcaagtatcctgattacctgaagatggtgagtgagccttatggcgacatgctgttcttttacctgcgcagggagcagatgttcgtgaggcatctgttcaaccgggccggaaccgtgggcgaaacagtgccagccgacctgtacatcaagggcaccaccgggaccctgcccagtacctcctattttcccacacctagcggctccatggtgacctccgacgcacagatctttaataagccctattggctgcagagagcccaggggcacaacaatgggatctgctggtctaaccagctgttcgtgaccgtggtggacaccactagaagtacaaatatgtctgtgtgcagcgccgtgagcaccagcgatagcacctacaagaatgacaatttcaaggaatacctgcgccacggagaagagtatgacctgcagtttattttccagctgtgtaaaatcaccttgaccgccgacgtgatgacatacatccacagcatgaatccatctatcctggaggactggaactttgggctgaccccccctcccagcggaacactcgaggatacatataggtacgtgacatcccaggcagtgacctgccagaagcccagcgcccccaagcccaaagacgaccctctgaaaaactataccttctgggaggtggacctgaaagagaagttttccgccgacctggatcagtttcctctgggaaggaagttcctgctgcaggctggcctgaaagctagacctaattttagactgggcaagagggccgcccccgccagtacctccaagaagtccagcaccaaaagacggaaggtgaaaagctga

SEQ ID NO: 69 - DNA, 1497 bp, synthetic construct
  source 1..1497: mol_type = other DNA
atgagcaacgaagccacagtgtacctgccacctgtgtccgtgtcaaaggtggtgagcacagacgagtacgtgactaggaccaacatctactaccacgccggttcctcaaggctgctggccgtgggacacccatactacgccattaagaagcaggattctaataagatcgctgtccctaaagtgtctggcctgcagtaccgcgtgtttagagtgaaactgcccgaccccaataagttcggctttcccgacacaagtttctacgaccctgcctctcagcgcctggtgtgggcttgcaccggcgtggaagtgggcaggggccagcccctgggagtgggcatttctgggcatcctctgctgaacaagctcgacgataccgaaaactctaacaagtacgtgggcaatagcggcaccgacaacagagagtgcatctccatggactataagcagacacagctgtgcctgatcgggtgtcgaccccccatcggagagcactgggggaagggaaccccctgtaatgccaaccaggtgaaggctggtgagtgtccccctctggagctgctgaataccgtgctgcaggacggcgacatggtggataccggctttggagccatggactttacaacactccaggccaacaagtccgatgtgcctctggacatctgcagcagcatctgcaagtaccctgattatctgaagatggtgtctgagccttacggggacatgctgtttttttatctgaggagagagcagatgtttgtgcgccatctgttcaatcgcgccggcaccgtgggagagacagtgcccgccgatctgtacatcaaaggcacaaccgggaccctgccatctacaagctatttccctacacctagcggctctatggtgactagcgacgcgcagatttttaacaagccctattggctgcagagggcccagggacacaataatggcatctgctggagcaatcagctgttcgtgactgtggtggacaccaccaggagcacaaatatgtcagtgtgctccgccgtgagcaccagcgacagcacctataagaatgacaacttcaaggagtacctgagacacggcgaggagtacgacctgcagttcatctttcagctgtgtaaaatcacactgaccgctgacgtgatgacatatattcatagtatgaaccctagcatcctggaagactggaacttcgggctgactccaccacccagcggcaccctggaggatacctacaggtatgtgacatctcaggccgtgacatgccagaagcctagcgcccctaagcccaaggatgatcccctgaagaattacaccttctgggaggtggacctgaaggaaaagttcagcgccgacctggaccagtttcccctgggccggaagttcctcctgcaggccggcctgaaggccaggccaaatttccggctggggaagagggctgcccctgccagcacctccaagaagtcttcaaccaagcgcaggaaagtgaagtcttga

SEQ ID NO: 70 - DNA, 1530 bp, synthetic construct
  source 1..1530: mol_type = other DNA
atgccaagcgatagtaccgtgtacctgccccccccaagtgtggctagggtggtgagcaccgatgactatgtgagccgcaccagcatcttttaccatgccggcagctctaggctgctgaccgtggggaacccctactttcgggtggtgcccaacggcgcaggcaacaaacaggccgtgccaaaggtgagcgcctatcagtacagggtgttcagagtcgctctgcccgaccccaataagttcgggctgccagatagcaccatctacaatcccgaaactcagcggctggtgtgggcctgtgtggggatggaaattggccggggccagccactggggatcgggctgagcggccaccctttctataacaaactggacgacacagagagcgcccacgccgctaccgccgttatcacccaggacgtgcgcgacaacgtcagtgtggactataagcagacacagctgtgcattctgggctgcgtgccagcaattggcgaacactgggctaaggggaccctgtgcaagcctgctcagctgcagccaggcgactgcccccctctggagctgaagaataccatcatcgaagatggagacatggtggacaccggctacggggctatggatttctccacactgcaggacacaaaatgtgaggtgcctctggatatctgccagtccatttgtaagtaccccgattacctgcagatgagcgccgacccctacggggactcaatgtttttttgcctgagaagggagcagctgttcgccaggcacttctggaacagggccggcgtgatgggggacacagtgcccaccgatctgtacatcaaaggaaccagcgccaacatgagagagaccccaggcagctgcgtgtacagccctagcccaagcgggagcatcatcacctccgattcccagctgttcaacaagccttattggctgcataaagctcagggacacaataatggcatttgttggcataaccagcttttcgtgactgtggtggacaccacaagatcaacaaacctgaccctgtgtgccagtacacagaatcccgtgccttctacctacgacccaactaagttcaagcagtacagcagacacgtggaggaatacgatctgcagttcatcttccagctgtgtacaatcacactgaccgccgaggtgatgagttacatccattctatgaattcaagcatcctggagaactggaatttcggcgtgccaccccctcccaccacaagcctggtggatacctatagattcgtgcagagcgtggccgtgacctgtcagaaggacacaacccctcccgagaagcaggatccatatgacaagctgaaattttggaccgtggacctgaaggagaaatttagctccgacctggaccagtatcctctggggagaaagtttctggtgcaggccggcctgaggagacggcccaccattggaccaagaaagaggccagctgcatccacatcaaccgcctctaccgcttctcggcctgccaagagggtgcggatcaggtccaagaagtga

SEQ ID NO: 71 - DNA, 1500 bp, synthetic construct
  source 1..1500: mol_type = other DNA
atgccctccgaggccaccgtgtacctgccacctgtgcccgtgagcaaggtggtgagcaccgacgagtacgtgagcaggaccagcatctactactacgccgggagttcccgcctgctgacagtggggcacccatatttttcaattaagaatacctcctctggcaacggcaagaaagtgctggtgcccaaggtcagcggcctgcagtaccgggtttttcgaatcaagctgcctgaccccaacaagtttggcttcccagacacctccttctacaacccagagacccagagactggtgtgggcttgcacaggcctggagattggcaggggtcagccacttggggtggggatcagcggccaccctctgctgaacaagtttgacgacaccgaaacatccaataagtacgctgggaagcccggcatcgacaaccgggagtgcctgtccatggattacaaacagacacagctgtgcatcctcggctgcaagcctccaatcggcgagcactggggaaaggggacaccctgcaacaacaacagcggcaatcctggagactgccctccgctgcagctgatcaacagcgtgattcaggacggggacatggtggataccgggttcggctgtatggactttaacaccctgcaggcaagcaagagcgatgtgccgattgacatctgttctagcgtgtgtaagtacccagactacctgcagatggcctccgagccctatggggactctctgttcttctttctgagaagggagcagatgttcgtgagacacttctttaaccgcgccggcaccctgggcgatccagtgcccggcgatctgtacattcaggggagcaacagcggcaataccgccaccgtgcagtccagcgctttttttcctacccctagcggctccatggtgacctctgagtctcagctgttcaataagccatactggctgcagcgggcccagggacacaacaacggcatttgctggggcaaccagctgtttgtgaccgtggtggacactacccgctctaccaacatgaccctgtgcgccgaagtgaagaaggaatccacctataaaaacgagaacttcaaggaatatcttagacatggggaggagtttgacctgcagtttattttccagctgtgcaagattacactgacagccgatgtgatgacctacatccacaagatggatgccaccatcctggaagactggcagtttggcctgactcccccccctagcgcctctctggaagatacctaccgcttcgtgactagcacagccatcacttgccagaagaacacccctcctaaaggcaaggaggaccccctgaaagactacatgttctgggaagtggatctgaaggagaagttcagcgccgacctggatcagttcccactgggcagaaagtttctgctgcaggctgggctgcaggccaggcccaagctgaaaagacctgctagcagcgcccctcgcacctcaaccaagaagaaaaaggtgaagagatga

SEQ ID NO: 72 - skipped

SEQ ID NO: 73 - DNA, 1503 bp, synthetic construct
  source 1..1503: mol_type = other DNA
atgtggcggcctagcgacagcacagtatatgtgcctcctcctaaccctgtatccaaagttgttgccacggatgcttatgttactcgcaccaacatattttatcatgccagcagttctagacttcttgcagtgggacatccttatttttccataaaacgggctaacaaaactgttgtgccaaaggtgtcaggatatcaatacagggtatttaaggtggtgttaccagatcctaacaaatttgcattgcctgactcgtctcttttcgatcccacaacacaacgtttagtatgggcatgcacaggcctagaggtgggcaggggacagccattaggtgtgggtgtaagtggacatcctttcctaaataaatatgatgatgttgaaaattcagggagtggtggtaaccctggacaggataacagggttaatgtaggtatggattataaacaaacacaattatgcatggttggatgtgccccccctttgggcgagcattggggtaaaggtaaacagtgtactaatacacctgtacaggctggtgactgcccgcccttagaacttattaccagtgttatacaggatggcgatatggttgacacaggctttggtgctatgaattttgctgatttgcagaccaataaatcagatgttcctattgacatatgtggcactacatgtaaatatccagattatttacaaatggctgcagacccatatggtgatagattatttttttttctacggaaggaacaaatgtttgccagacatttttttaacagggctggcgaggtgggggaacctgtgcctgatacacttataattaagggtagtggaaatcgcacgtctgtagggagtagtatatatgttaacaccccgagcggctctttggtgtcctctgaggcacaattgtttaataagccatattggctacaaaaagcccagggacataacaatggtatttgttggggtaatcaactgtttgttactgtggtagataccacacgcagtaccaacatgacattatgtgcatccgtaactacatcttccacatacaccaattctgattataaagagtacatgcgtcatgtggaagagtatgatttacaatttatttttcaattatgtagcattacattgtctgctgaagtaatggcctatattcacacaatgaatccctctgttttggaagactggaactttgggttatcgcctcccccaaatggtacattagaagatacctataggtatgtgcagtcacaggccattacctgtcaaaagcccactcctgaaaaggaaaagccagatccctataagaaccttagtttttgggaggttaatttaaaagaaaagttttctagtgaattggatcagtatcctttgggacgcaagtttttgttacaaagtggatataggggacggtcctctattcgtacaggtgttaagcgccctgctgtttccaaagcctctgctgcccctaaacgtaagcgcgccaaaactaaaaggtaa

SEQ ID NO: 74 - DNA, 1518 bp, synthetic construct
  source 1..1518: mol_type = other DNA
atgtctctttggctgcctagtgaggccactgtctacttgcctcctgtcccagtatctaaggttgtaagcacggatgaatatgttgcacgcacaaacatatattatcatgcaggaacatccagactacttgcagttggacatccctattttcctattaaaaaacctaacaataacaaaatattagttcctaaagtatcaggattacaatacagggtatttagaatacatttacctgaccccaataagtttggttttcctgacacctcattttataatccagatacacagcggctggtttgggcctgtgtaggtgttgaggtaggtcgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaatcggttgcaaaccacctataggggaacactggggcaaaggatccccatgtaccaatgttgcagtaaatccaggtgattgtccaccattagagttaataaacacagttattcaggatggtgatatggttgatactggctttggtgctatggactttactacattacaggctaacaaaagtgaagttccactggatatttgtacatctatttgcaaatatccagattatattaaaatggtgtcagaaccatatggcgacagcttatttttttatttacgaagggaacaaatgtttgttagacatttatttaatagggctggtgctgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaaccccccccaggaggcacactagaagatacttataggtttgtaacatcccaggcaattgcttgtcaaaaacatacacctccagcacctaaagaagatccccttaaaaaatacactttttgggaagtaaatttaaaggaaaagttttctgcagacctagatcagtttcctttaggacgcaaatttttactacaagcaggattgaaggccaaaccaaaatttacattaggaaaacgaaaagctacacccaccacctcatctacctctacaactgctaaacgcaaaaaacgtaagctgtaa

SEQ ID NO: 75 - DNA, 1512 bp, synthetic construct
  source 1..1512: mol_type = other DNA
atgcctagtgacaataccgtatatcttccacctccttctgtggcaagagttgtaaataccgatgattatgtgactcccacaagcatattttatcatgctggcagctctagattattaactgttggtaatccatattttagggttcctgcaggtggtggcaataagcaggatattcctaaggtttctgcataccaatatagagtatttagggtgcagttacctgacccaaataaatttggtttacctgatactagtatttataatcctgaaacacaacgtttagtgtgggcctgtgctggagtggaaattggccgtggtcagcctttaggtgttggccttagtgggcatccattttataataaattagatgacactgaaagttcccatgccgccacgtctaatgtttctgaggacgttagggacaatgtgtctgtagattataagcagacacagttatgtattttgggctgtgcccctgctattggggaacactgggctaaaggcactgcttgtaaatcgcgtcctttatcacagggcgattgcccccctttagaacttaaaaacacagttttggaagatggtgatatggtagatactggatatggtgccatggactttagtacattgcaagatactaaatgtgaggtaccattggatatttgtcagtctatttgtaaatatcctgattatttacaaatgtctgcagatccttatggggattccatgtttttttgcttacggcgtgagcagctttttgctaggcatttttggaatagagcaggtactatgggtgacactgtgcctcaatccttatatattaaaggcacaggtatgcgtgcttcacctggcagctgtgtgtattctccctctccaagtggctctattgttacctctgactcccagttgtttaataaaccatattggttacataaggcacagggtcataacaatggtgtttgctggcataatcaattatttgttactgtggtagataccactcgcagtaccaatttaacaatatgtgcttctacacagtctcctgtacctgggcaatatgatgctaccaaatttaagcagtatagcagacatgttgaggaatatgatttgcagtttatttttcagttgtgtactattactttaactgcagatgttatgtcctatattcatagtatgaatagcagtattttagaggattggaactttggtgttccccccccgccaactactagtttggtggatacatatcgttttgtacaatctgttgctattacctgtcaaaaggatgctgcaccggctgaaaataaggatccctatgataagttaaagttttggaatgtggatttaaaggaaaagttttctttagacttagatcaatatccccttggacgtaaatttttggttcaggctggattgcgtcgcaagcccaccataggccctcgcaaacgttctgctccatctgccactacgtcttctaaacctgccaagcgtgtgcgtgtacgtgccaggaagtaa

SEQ ID NO: 76 - DNA, 1515 bp, synthetic construct; identical to SEQ ID NO: 66
  source 1..1515: mol_type = other DNA

SEQ ID NO: 77 - DNA, 1488 bp, synthetic construct
  source 1..1488: mol_type = other DNA
atgcctagtgaggccacagtgtacctgcctcctgtacctgtatctaaagttgtcagcactgatgaatatgtgtctcgcacaagcatttattattatgctggtagttccagacttcttgctgttggccatccatatttttctattaaaaatcctactaacgctaaaaaattattggtacccaaagtatcaggcttgcaatatagggtttttagggtccgtttaccagatcctaataaatttggatttcctgacacctccttttataaccctgatacacaacgattagtatgggcatgtgtaggccttgaaataggtagagggcagccattaggcgttggcataagtggtcatcctttattaaacaaatttgatgacactgaaaccggtaacaagtatcctggacaaccgggtgctgataatagggaatgtttatccatggattataaacaaacacagttatgtttacttggatgtaagcctccaacaggggaacattggggtaaaggtgttgcttgtactaatgcagcacctgccaatgattgtccacctttagaacttataaatactattattgaggatggtgatatggtggacacaggatttggttgcatggattttaaaacattgcaggctaataaaagtgatgttcctattgatatttgtggcagtacatgcaaatatccagattatttaaaaatgactagtgagccttatggtgatagtttatttttctttcttcgacgtgaacaaatgtttgtaagacacttttttaatagggctggtacattaggagaggctgttcccgatgacctgtacattaaaggttcaggaactactgcctctattcaaagcagtgctttttttcccactcctagtggatcaatggttacttccgaatctcagttatttaataagccatattggctacaacgtgcacaaggtcataataatggtatttgttggggcaatcaggtatttgttactgtggtagataccactcgcagtactaatatgactttatgcacacaagtaactagtgacagtacatataaaaatgaaaattttaaagaatatataagacatgttgaagaatatgatctacagtttgtttttcaactatgcaaagttaccttaactgcagaagttatgacatatattcatgctatgaatccagatattttagaagattggcaatttggtttaacacctcctccatctgctagtttacaggatacctataggtttgttacctctcaggctattacgtgtcaaaaaacagtacctccaaaggaaaaggaagaccccttaggtaaatatacattttgggaagtggatttaaaggaaaaattttcagcagatttagatcagtttcctttgggacgcaagtttttattacaggcaggtcttaaagcaaaacctaaacttaaacgtgcagcccccacatccacccgcacatcgtctgcaaaacgcaaaaaggttaaaaaataa

SEQ ID NO: 78 - DNA, 1509 bp, synthetic construct
  source 1..1509: mol_type = other DNA
atgtctctgtggcggtctaacgaagccactgtctacctgcctccagtgtcagtgtctaaggttgttagcactgatgaatatgtaacacgcacaaacatctactatcatgcaggcagttctaggctattagctgtgggtcacccatactatgctattaaaaaacaagattctaataaaatagcagtacccaaggtatctggtttgcaatacagagtatttagagtaaaattaccagatcctaataagtttggatttccagacacatcattttatgatcctgcctcccagcgtttagtttgggcctgtacaggagttgaagtaggtcgcggtcagccattaggtgtaggtattagtggtcatcctttattaaataaattggatgatactgaaaattctaataaatatgttggtaactctggtacagataacagggaatgcatttctatggattataaacaaacacaattgtgtttaataggttgtaggcctcctataggtgaacattggggaaaaggcacaccttgtaatgctaaccaggtaaaagcaggagaatgtcctcctttggagttactaaacactgtactacaagacggggacatggtagacacaggatttggtgcaatggattttactacattacaagctaataaaagtgatgttcccctagatatatgcagttccatttgcaaatatcctgattatctaaaaatggtttctgagccatatggagatatgttatttttttatttacgtagagagcaaatgtttgttagacatttatttaatagggctggaactgtaggtgaaacagtacctgcagacctatatattaagggtaccactggcacattgcctagtactagttattttcctactcctagtggctctatggtaacctcagatgcacaaatatttaataaaccatattggttgcaacgtgcacaaggccataataatggtatttgttggagtaaccaattgtttgttactgtagttgatacaacccgtagtacaaatatgtctgtgtgttctgctgtgtctactagtgacagtacatataaaaatgacaattttaaggaatatttaaggcatggtgaagaatatgatttacagtttatttttcagttatgtaaaataacactaacagcagatgttatgacatatattcatagtatgaacccgtccattttagaggattggaattttggccttacaccaccgccttctggtaccttagaggacacatatcgctatgtaacatcacaggctgtaacttgtcaaaaacccagtgcaccaaaacctaaagatgatccattaaaaaattatactttttgggaggttgatttaaaggaaaagttttctgcagacttagatcaatttccgttgggccgtaaatttttgttacaagcaggactaaaggccaggcctaattttagattaggcaagcgtgcagctccagcatctacatctaaaaaatcttctactaaacgtagaaaagtaaaaagttaa

SEQ ID NO: 79 - DNA, 1497 bp, synthetic construct
  source 1..1497: mol_type = other DNA
atgtctaacgaagccactgtctacctgcctccagtgtcagtgtctaaggttgttagcactgatgaatatgtaacacgcacaaacatctactatcatgcaggcagttctaggctattagctgtgggtcacccatactatgctattaaaaaacaagattctaataaaatagcagtacccaaggtatctggtttgcaatacagagtatttagagtaaaattaccagatcctaataagtttggatttccagacacatcattttatgatcctgcctcccagcgtttagtttgggcctgtacaggagttgaagtaggtcgcggtcagccattaggtgtaggtattagtggtcatcctttattaaataaattggatgatactgaaaattctaataaatatgttggtaactctggtacagataacagggaatgcatttctatggattataaacaaacacaattgtgtttaataggttgtaggcctcctataggtgaacattggggaaaaggcacaccttgtaatgctaaccaggtaaaagcaggagaatgtcctcctttggagttactaaacactgtactacaagacggggacatggtagacacaggatttggtgcaatggattttactacattacaagctaataaaagtgatgttcccctagatatatgcagttccatttgcaaatatcctgattatctaaaaatggtttctgagccatatggagatatgttatttttttatttacgtagagagcaaatgtttgttagacatttatttaatagggctggaactgtaggtgaaacagtacctgcagacctatatattaagggtaccactggcacattgcctagtactagttattttcctactcctagtggctctatggtaacctcagatgcacaaatatttaataaaccatattggttgcaacgtgcacaaggccataataatggtatttgttggagtaaccaattgtttgttactgtagttgatacaacccgtagtacaaatatgtctgtgtgttctgctgtgtctactagtgacagtacatataaaaatgacaattttaaggaatatttaaggcatggtgaagaatatgatttacagtttatttttcagttatgtaaaataacactaacagcagatgttatgacatatattcatagtatgaacccgtccattttagaggattggaattttggccttacaccaccgccttctggtaccttagaggacacatatcgctatgtaacatcacaggctgtaacttgtcaaaaacccagtgcaccaaaacctaaagatgatccattaaaaaattatactttttgggaggttgatttaaaggaaaagttttctgcagacttagatcaatttccgttgggccgtaaatttttgttacaagcaggactaaaggccaggcctaattttagattaggcaagcgtgcagctccagcatctacatctaaaaaatcttctactaaacgtagaaaagtaaaaagttaa

SEQ ID NO: 80 - DNA, 1530 bp, synthetic construct
  source 1..1530: mol_type = other DNA
atgcctagtgacagtacggtatatcttccaccaccttctgtggccagagttgtcagcactgatgattatgtgtctcgcacaagcatattttatcatgcaggcagttcccgattattaactgtaggcaatccatattttagggttgtacctaatggtgcaggtaataaacaggctgttcctaaggtatccgcatatcagtatagggtgtttagagtagctttacccgatcctaataaatttggattacctgattctactatatataatcctgaaacacaacgtttggtttgggcatgtgtaggtatggaaattggtcgtgggcagcctttaggtattggcctaagtggccatccattttataataaattggatgatacagaaagtgctcatgcagctacagctgttattacgcaggatgttagggataatgtgtcagttgattataagcaaacacagctgtgtattttaggttgtgtacctgctattggtgagcactgggccaagggcacactttgtaaacctgcacaattgcaacctggtgactgtcctcctttggaacttaaaaacaccattattgaggatggtgatatggtggatacaggttatggggcaatggattttagtacattgcaggatacaaagtgcgaggttccattagacatttgtcaatccatctgtaaatatccagattatttgcaaatgtctgctgatccctatggggattctatgtttttttgcctacgccgtgaacaactgtttgcaagacatttttggaatagggcaggtgttatgggtgacacagtacctacggacctatatattaaaggcactagcgctaatatgcgtgaaacccctggcagttgtgtgtattccccttctcccagtggctctattattacttctgattctcaattatttaataagccatattggttacataaggcccagggccataacaatggtatttgttggcataatcagttgtttgttactgtagtggacactacccgcagtactaatttaacattatgtgcctctacacaaaatcctgtgccaagtacatatgaccctactaagtttaagcagtatagtagacatgtggaggaatatgatttacagtttatttttcagttgtgcactattactttaactgcagaggttatgtcatatatccatagtatgaatagtagtatattagaaaattggaattttggtgtccctccaccacctactacaagtttggtggatacatatcgttttgtgcaatcagttgctgttacctgtcaaaaggatactacacctccagaaaagcaggatccatatgataaattaaagttttggactgttgacctaaaggaaaaattttcctccgatttggatcaatatccccttggtcgaaagtttttagttcaggctgggttacgtcgtaggcctaccataggacctcgtaagcgtcctgctgcttccacgtctactgcatctactgcatctaggcctgccaaacgtgtacgtatacgtagtaagaaataa

SEQ ID NO: 81 - DNA, 1500 bp, synthetic construct
  source 1..1500: mol_type = other DNA
atgcctagtgaggccactgtgtacctgcctcctgtacctgtctctaaggttgtaagcactgatgagtatgtgtctcgcacaagcatctattattatgcaggcagttctcgattactaacagtaggacatccctatttttctattaaaaacaccagtagtggtaatggtaaaaaagttttagttcccaaggtgtctggcctgcaatacagggtatttagaattaaattgccggaccctaataaatttggttttccagatacatctttttataacccagaaacccaaaggttggtgtgggcctgtacaggcttggaaattggtaggggacagcctttaggtgtgggtattagtgggcatcctttattaaacaagtttgatgatactgaaaccagtaacaaatatgctggtaaacctggtatagataatagggaatgtttatctatggattataagcagactcagttatgcattttaggatgcaaacctcctataggtgaacattggggtaagggaaccccttgtaataataattcaggaaatcctggggattgtcctcccctacagctcattaacagtgtaatacaggatggggacatggtagatacaggatttggttgcatggattttaataccttgcaagctagtaaaagtgatgtgcccattgatatatgtagcagtgtatgtaagtatccagattatttgcaaatggctagcgagccatatggtgacagtttgttcttttttcttagacgtgagcaaatgtttgttagacacttttttaatagggccggtaccttaggtgaccctgtgccaggtgatttatatatacaagggtctaactctggcaatactgccactgtacaaagcagtgctttttttcctactcctagtggttctatggtaacctcagaatcccaattatttaataaaccgtactggttacaacgtgcgcagggccacaataatggcatatgttggggcaatcagttgtttgtcacagttgtggataccactcgtagcactaacatgactttatgtgctgaggttaaaaaggaaagcacatataaaaatgaaaattttaaggaataccttcgtcatggcgaggaatttgatttacaatttatttttcaattgtgcaaaattacattaacagctgatgttatgacatacattcataagatggatgccactattttagaggactggcaatttggccttaccccaccaccgtctgcatctttggaggacacatacagatttgtcacttctactgctataacttgtcaaaaaaacacaccacctaaaggaaaggaagatcctttaaaggactatatgttttgggaggtggatttaaaagaaaagttttctgcagatttagatcagtttcctttaggtaggaagtttttgttacaggcagggctacaggctaggcccaaactaaaacgccctgcatcatcggccccacgtacctccacaaagaagaaaaaggttaaaaggtaa

SEQ ID NO: 82 - DNA, 1485 bp, synthetic construct
  source 1..1485: mol_type = other DNA
atgcctagtgaggccactgtgtacctgcctcctgtgcctgtgtctaaggttgtaagcactgatgaatatgtgtcacgcacaagcatttattattatgctggcagttccagacttttggctgttggcaatccatatttttccatcaaaagtcccaataacaataaaaaagtattagttcccaaggtatcaggcttacagtatagggtctttagggtgcgtttacctgatcccaataaatttggttttcctgatacatctttttataaccctgatacacaacgtttggtctgggcatgtgtaggccttgaaataggtaggggacagccattgggtgttggcgtaagtggtcatccttatttaaataaatttgatgacactgaaaccagtaacagatatcccgcacagccagggtctgataacagggaatgcttatctatggattataaacaaacacaattatgtttaattggctgtaaacctcccactggtgagcattggggtaaaggtgttgcctgtaacaataatgcagctgctactgattgtcctccattggaactttttaattctattattgaggatggtgacatggtagatacagggtttggatgcatggactttggtacattgcaggctaataaaagtgatgtgcctattgatatttgtaacagtacatgcaaatatccagattatttaaaaatggccagtgaaccttatggggatagtttgttcttttttcttagacgtgagcagatgtttgttagacacttttttaatagggctggaaaacttggcgaggctgtcccggatgacctttatattaaagggtccggtaatactgcagttatccaaagtagtgcattttttccaactcctagtggctctatagttacctcagaatcacaattatttaataagccttattggctacagcgtgcacaaggtcataacaatggcatttgctggggcaatcagttatttgttaccgtggttgataccactcgtagcactaatatgacattatgcactgaagtaactaaggaaggtacatataaaaatgataattttaaggaatatgtacgtcatgttgaagaatatgacttacagtttgtttttcagctttgcaaaattacactaactgcagagataatgacatatatacatactatggattccaatattttggaggactggcaatttggtttaacacctcctccgtctgccagtttacaggacacatatagatttgttacctcccaggctattacttgccaaaaaacagcaccccctaaagaaaaggaagatccattaaataaatatactttttgggaggttaacttaaaggaaaagttttctgcagatctagatcagtttcctttgggacgaaagtttttattacaatcaggccttaaagcaaagcccagactaaaacgttcggcccctactacccgtgcaccatccaccaaacgcaaaaaggttaaaaaataa

(19) United States
(12) Patent Application Publication
(10) Pub. No.: US 2025/0281594 A1
(43) Pub. Date: Sep. 11, 2025

(54) TRUNCATED RECOMBINANT L1 PROTEIN OF HUMAN PAPILLOMAVIRUS

(71) Applicants: SK bioscience Co., Ltd., Gyeonggi-do (KR); Sunflower Therapeutics, PBC, Hingham, MA (US)

(72) Inventors: Kyung-Ho HA, Gyeonggi-do (KR); Yeon-Tae Kim, Gyeonggi-do (KR); Gee-In Jung, Gyeonggi-do (KR); Jin-Kyoo Chung, Gyeonggi-do (KR); Na-Yeon Kim, Gyeonggi-do (KR); Laura Crowell, Hingham, MA (US); Minh Le, Hingham, MA (US); Michael Sheets, Hingham, MA (US)

(21) Appl. No.: 19/074,708

(22) Filed: Mar. 10, 2025

Related U.S. Application Data
(60) Provisional application No. 63/563,554, filed on Mar. 11, 2024.

(51) Int. Cl. A61K 39/12; A61K 39/00; A61P 31/16; C07K 14/005; C12N 7/00; C12N 15/81

(52) U.S. Cl. CPC A61K 39/12; A61P 31/16; C07K 14/005; C12N 7/00; C12N 15/815; A61K 2039/545; A61K 2039/55505; C12N 2710/20022; C12N 2710/20023; C12N 2710/20034; C12N 2710/20051

(57) ABSTRACT

The disclosure relates to a truncated recombinant L1 protein of Human papillomavirus (HPV), a recombinant polynucleotide encoding the protein, a recombinant vector comprising the polynucleotide, an isolated cell comprising the recombinant vector, a composition comprising the protein, and a method for producing the protein. The disclosure also relates to an HPV virus-like particle (VLP) comprising the protein, an immunogenic composition comprising the protein or the VLP, and a method for inducing an immune response to HPV in an individual.

### CROSS-REFERENCE TO RELATED APPLICATIONS

This application claims the benefit of and priority to U.S. Provisional Application No. 63/563,554, filed Mar. 11, 2024, the entire contents of which are incorporated herein by reference.

### FIELD OF THE DISCLOSURE

The invention relates to a truncated recombinant L1 protein of Human papillomavirus (HPV), a recombinant polynucleotide encoding the protein, a recombinant vector comprising the polynucleotide, an isolated cell comprising the recombinant vector, a composition comprising the protein, and a method for producing the protein. The disclosure also relates to an HPV virus-like particle (VLP) comprising the protein, an immunogenic composition comprising the protein or the VLP, and a method for inducing an immune response to HPV in an individual.

### DESCRIPTION OF THE TEXT FILE SUBMITTED ELECTRONICALLY

The instant application contains a sequence listing, which has been submitted in XML format via PatentCenter. The XML file named, “OPP20250,645US_Sequence_Listing.xml,” was created on Feb. 25, 2025, and is approximately 162,315 bytes in size, the entire contents of which are incorporated herein by reference.

### BACKGROUND OF THE DISCLOSURE

Human papillomavirus (HPV) is a group of more than 200 viruses that can infect the skin and mucous membranes. HPV can cause a variety of health problems, including genital warts, cervical cancer, and other cancers. HPV is spread through skin-to-skin contact, usually during sexual activity. The virus can also be spread through oral sex and other forms of close contact. There is no cure for HPV, but there are treatments available for the symptoms of HPV infection. Because there is no cure for HPV, vaccination is the best way to protect the body from HPV infection and the diseases that HPV can cause. The HPV vaccine is highly effective at preventing HPV infection, and it can also help to prevent the development of cervical cancer.

Currently, there are over 90 different types of HPV, mainly causing papillary disease in the skin and mucosa of humans. HPV types are divided into three groups depending on their relation with tumorigenesis: (1) group of low or no cancerogenic risk, containing types 6 and 11; (2) group of medium cancerogenic risk, containing types 22, 33, 35, 39, 52, 56, 58, 59 and 68; and (3) group of high cancerogenic risk, containing types 16, 18, 31 and 45.

Human papillomavirus (HPV) is a non-enveloped, double-stranded DNA virus with a circular genome approximately 7.2-8 kb in length. It contains eight open reading frames (ORFs) that encode for various proteins involved in viral replication, transcription, and transformation. The genome can be divided into three regions:
(1) Early region (E): Approximately 4.5 kb long, encodes six non-structural proteins (E1, E2, E4-E7) involved in viral replication, transcription, and transformation.
(2) Late region (L): Approximately 2.5 kb long, encodes the major capsid protein L1 and the minor capsid protein L2, which form the viral particle's outer shell.
(3) Long control region (LCR): Located between the L region and the E region's initiation point, approximately 800-900 bp long, contains regulatory elements for DNA replication and expression instead of coding for proteins.

Viral particles are 45-55 nm in diameter, with an icosahedral nucleocapsid composed of L1 and L2 proteins. The nucleocapsid protects the viral DNA within the particle.

HPV L1 protein, with a molecular weight of 55-60 kDa, is the major capsid protein of the human papillomavirus and the main protein comprising the HPV vaccine. HPV L1 protein expressed in multiple different expression systems can form Virus-like particles (VLPs) which resemble native HPV particles morphologically, without the assistance of the L2 protein. The VLP, consisting of 72 pentamers of the L1 proteins, exhibits icosahedral symmetry. Since the VLPs retain the native epitopes of the viral particles, they are highly immunogenic and can induce the generation of neutralizing antibodies against homologous HPV (Kirnbauer, R. F. Booy, et al. 1992 Proc Natl Acad Sci USA 89 (24): 12180-4). Furthermore, the VLPs are safe and have no potential cancerogenic risk as they contain no viral DNA. Therefore, VLP vaccines become the primary candidate for an HPV vaccine.

The key for development of a vaccine is to efficiently produce VLPs of HPV L1 at large-scale. Currently, the most commonly used expression systems are eukaryotic expression systems including mammalian cells, insect cells and yeast. HPV L1 protein expressed in eukaryotic systems shows little conformational difference from that of the native virus, and can self-assemble into VLPs. These VLPs are typically expressed recombinantly at low levels in the named host organisms, requiring purification via gradient density centrifugation, which is time consuming, low yielding and not relevant for commercial scale manufacturing.

These manufacturing challenges impact the volumetric productivities of commercial processes, leading to high costs of goods manufactured (COGsM), that are passed on to the patient consumer. The existing price of marketed HPV vaccine products substantially limits their accessibility and uptake with global patients that would benefit from this life-saving product.

Therefore, there is a continued need for the development of highly manufacturable HPV L1 proteins that retain the ability to form uniform virus-like particles (VLPs) and can thereby induce the production of protective antibodies when these particles are delivered in vivo. This necessitates the advancement of efficient and cost-effective technologies for the production of high quality HPV L1 proteins.

### SUMMARY OF THE DISCLOSURE

Accordingly, the present disclosure provides, inter alia, compositions and methods that find use in more efficient vaccination approaches against HPV.

An embodiment described herein provides a truncated recombinant human papillomavirus (HPV) L1 protein with at least 2 amino acids deleted from positions 2 to 66, or positions corresponding thereto, of the N-terminus.

Another embodiment described herein provides a recombinant polynucleotide encoding the truncated recombinant HPV L1 protein.

Another embodiment described herein provides a recombinant vector comprising the polynucleotide.

Another embodiment described herein provides an isolated cell comprising the recombinant vector.

Another embodiment described herein provides a composition comprising the truncated recombinant HPV L1 protein.

Another embodiment described herein provides a recombinant HPV virus-like particle (VLP) comprising the truncated recombinant HPV L1 protein.

Another embodiment described herein provides an immunogenic composition comprising the truncated recombinant HPV L1 protein or the VLP comprising the same, and optionally comprising pharmaceutically acceptable carriers, excipients and/or adjuvant.

Another embodiment described herein provides a method for inducing an immune response to HPV in an individual, comprising administering the immunogenic composition to an individual.

Another embodiment described herein provides a method for preventing an HPV infection or a disease associated with an HPV infection, comprising administering the immunogenic composition to a subject in need thereof. Another embodiment described herein provides a method for producing the truncated recombinant HPV L1 protein, comprising expressing a polynucleotide encoding the HPV L1 protein in a yeast cell.

### BRIEF DESCRIPTION OF THE DRAWINGS

FIG. 1: Exemplary expression vector.

FIG. 2: Western Blot demonstrating expression of HPV 6 L1 in K. phaffii.

FIG. 3: Western Blot demonstrating expression of HPV 11 L1 in K. phaffii FIG. 4: Western Blot demonstrating expression of HPV 16 L1 in K. phaffii.

FIG. 5: Western Blot demonstrating expression of HPV 18 L1 in K. phaffii.

FIG. 6: Western Blot demonstrating expression of HPV 31 L1 in K. phaffii.

FIG. 7: Western Blot demonstrating expression of HPV 33 L1 in K. phaffii.

FIG. 8: Western Blot demonstrating expression of HPV 35 L1 in K. phaffii.

FIG. 9: Western Blot demonstrating expression of HPV 45 L1 in K. phaffii.

FIG. 10: Western Blot demonstrating expression of HPV 52 L1 in K. phaffii.

FIG. 11: Western Blot demonstrating expression of HPV 58 L1 in K. phaffii.

FIG. 12: Western Blot demonstrating expression of HPV 16 L1 in K. phaffii with different codon optimization.

FIG. 13: TEM observation of HPV virus-like particles (VLPs).

FIG. 14: A) Relative total antibody titers following administration of HPV 16 L1 VLPs to mice in as-is or N-terminal truncated form, as assessed by ELISA on mouse serum. B) Relative neutralizing antibody titer following administration of HPV 16 L1 VLPs to mice in as-is or N-terminal truncated form, as determined by PBNA on mouse serum.

FIG. 15: A) Relative total antibody titers following administration of various HPV L1 VLPs to mice in as-is or N-terminal truncated form, as assessed by ELISA on mouse serum. B) Relative neutralizing antibody titer following administration of various HPV L1 VLPs to mice in as-is or N-terminal truncated form, as determined by PBNA on mouse serum.

### DETAILED DESCRIPTION OF THE DISCLOSURE

According to one aspect of the present disclosure, there is provided a truncated recombinant human papillomavirus (HPV) L1 protein with at least 2 amino acids deleted from positions 2 to 66, or positions corresponding thereto, of the N-terminus.

In one embodiment, the HPV L1 protein may have a deletion of amino acids at positions 2-3, 2-5, 2-7, 2-10, 2-15, 2-17, 2-20, 2-22, 2-31, or 2-66, or positions corresponding thereto, of N-terminus.

In one embodiment, the HPV may be an HPV serotype selected from HPV serotypes 6, 11, 16, 18, 31, 33, 35, 45, 52, and 58.

In one embodiment, the HPV6 L1 protein may have a deletion of amino acids at positions 2-3 or 2-5, or positions corresponding thereto, of N-terminus; the HPV11 L1 protein may have a deletion of amino acids at positions 2-3 or positions corresponding thereto, of N-terminus; the HPV16 L1 protein may have a deletion of amino acids at positions 2-5, 2-10, 2-15, or 2-20, or positions corresponding thereto, of N-terminus; the HPV18 L1 protein may have a deletion of amino acids at positions 2-5, 2-7, or 2-66, or positions corresponding thereto, of N-terminus; the HPV31, HPV33 or HPV35 L1 protein may have a deletion of amino acids at positions 2-5, or positions corresponding thereto, of N-terminus; the HPV45 or HPV58 L1 protein may have a deletion of amino acids at positions 2-10, 2-17, 2-22, or 2-31, or positions corresponding thereto, of N-terminus; or the HPV52 L1 protein may have a deletion of amino acids at positions 2-31, or positions corresponding thereto, of N-terminus.

In one embodiment, the HPV L1 protein may comprise of an amino acid sequence selected from one of SEQ ID NOs: 21-41, or a variant having at least about 90%, or at least about 95%, or at least about 98%, at least about 99% identity to one of SEQ ID NOs: 21-41.

In one embodiment, the HPV L1 protein may consist of an amino acid sequence selected from one of SEQ ID NOs: 21-41.

According to another aspect of the present disclosure, there is provided a recombinant polynucleotide encoding the truncated recombinant HPV L1 protein.

In one embodiment, the polynucleotide may be codon-optimized for expression in a yeast cell.

In one embodiment, the polynucleotide may comprise of a nucleotide sequence selected from one of SEQ ID NOs: 42-62, or a variant having at least about 60%, at least about 70%, at least about 80%, at least about 90%, or at least about 95%, or at least about 98%, at least about 99% identity to one of SEQ ID NOs: 42-62.

In one embodiment, the polynucleotide may consist of a nucleotide sequence selected from one of SEQ ID NOs: 42-62.

According to another aspect of the present disclosure, there is provided a recombinant vector comprising the polynucleotide.

According to another aspect of the present disclosure, there is provided an isolated cell comprising the recombinant vector.

According to another aspect of the present disclosure, there is provided a composition comprising the truncated recombinant HPV L1 protein. There also is provided a composition comprising the polynucleotide encoding the truncated recombinant HPV L1 protein, vector comprising the polynucleotide, or the cell comprising the recombinant vector.

According to another aspect of the present disclosure, there is provided a recombinant HPV virus-like particle (VLP) comprising the truncated recombinant HPV L1 protein.

According to another aspect of the present disclosure, there is provided an immunogenic composition comprising the truncated recombinant HPV L1 protein or the VLP comprising the same, and optionally comprising pharmaceutically acceptable carriers, excipients and/or adjuvant.

In one embodiment, the immunogenic composition may be used for inducing an immune response to HPV in an individual.

In one embodiment, the immunogenic composition may be used for the prevention of HPV infection or a disease associated with HPV infection.

In one embodiment, the disease associated with HPV infection may be condyloma acuminatum, genital warts, cervical neoplasia, or cervical cancer.

According to another aspect of the present disclosure, there is provided a method for inducing an immune response to HPV in an individual, comprising administering the immunogenic composition to an individual.

According to another aspect of the present disclosure, there is provided a method for preventing an HPV infection or a disease associated with an HPV infection, comprising administering the immunogenic composition to a subject in need thereof. In one embodiment, the disease associated with HPV infection is condyloma acuminatum, genital warts, cervical neoplasia, or cervical cancer. In one embodiment, the subject is likely to be exposed or has been recently exposed to HPV. In one embodiment, the subject is a human aged 9 to 45 years. Preferably, the subject is a female aged 9 to 45 years, and more preferably, the subject is a female aged 26 years or younger.

According to another aspect of the present disclosure, there is provided a method for producing the truncated recombinant HPV L1 protein, comprising expressing a polynucleotide encoding the HPV L1 protein in a yeast cell.

Hereinafter, the present disclosure will be described in more detail.

Where the terms “comprise”, “comprises”, “comprised” or “comprising” are used in this specification (including the claims) they are to be interpreted as specifying the presence of the stated features, integers, steps or components, but not precluding the presence of one or more other feature, integer, step, component or group thereof.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present disclosure before the priority date of each claim of this application.

The disclosure is at least partially based on the discovery that a truncated recombinant HPV L1 protein capable of inducing the generation of neutralization antibodies against HPV can be expressed in a yeast expression system with high expression levels, and the truncated HPV L1 protein can be produced with a high yield.

Therefore, in one aspect, the disclosure relates to a truncated recombinant HPV L1 protein with at least 2 amino acids deleted from positions 2 to 66, or positions corresponding thereto, of the N-terminus.

The term “a truncated HPV L1 protein” refers to the protein with one or more amino acids deleted at the N- and/or C-terminal of wild-type HPV L1 protein, wherein the example of the wild-type HPV L1 protein includes, but is not limited to, the full-length L1 proteins such as SEQ ID NOs: 1-10.

In a preferred embodiment, the truncated HPV L1 protein has a deletion of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 amino acids from positions 2 to 66, or positions corresponding thereto, of the N-terminus, as compared with wild type HPV L1 protein.

In another preferred embodiment, the truncated HPV L1 protein has a deletion of amino acids at positions 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-49, 2-50, 2-51, 2-52, 2-53, 2-54, 2-55, 2-56, 2-57, 2-58, 2-59, 2-60, 2-61, 2-62, 2-63, 2-64, 2-65, or 2-66, or positions corresponding thereto, of N-terminus, as compared with wild type HPV L1 protein. In an exemplary embodiment, the truncated HPV L1 protein may haves a deletion of amino acids at positions 2-3, 2-5, 2-7, 2-10, 2-15, 2-17, 2-20, 2-22, 2-31, or 2-66 of N-terminus, as compared with wild type HPV L1 protein.

In another preferred embodiment, the HPV may be an HPV serotype of low cancerogenic risk, medium cancerogenic risk, or high cancerogenic risk. For example, the HPV may be an HPV serotype such as HPV6, 11, 16, 18, 22, 23, 31, 33, 35, 39, 45, 52, 56, 58, 59, 68, etc., but is not limited thereto. In an exemplary embodiment, the HPV may be an HPV serotype selected from HPV serotypes 6, 11, 16, 18, 31, 33, 35, 45, 52, and 58.

In another preferred embodiment, the HPV6 L1 protein may have a deletion of amino acids at positions 2-3 or 2-5, or positions corresponding thereto, of N-terminus; the HPV11 L1 protein may have a deletion of amino acids at positions 2-3 or positions corresponding thereto, of N-terminus; the HPV16 L1 protein may have a deletion of amino acids at positions 2-5, 2-10, 2-15, or 2-20, or positions corresponding thereto, of N-terminus; the HPV18 L1 protein may have a deletion of amino acids at positions 2-5, 2-7, or 2-66, or positions corresponding thereto, of N-terminus; the HPV31, HPV33 or HPV35 L1 protein may have a deletion of amino acids at positions 2-5, or positions corresponding thereto, of N-terminus; the HPV45 or HPV58 L1 protein may have a deletion of amino acids at positions 2-10, 2-17, 2-22, or 2-31, or positions corresponding thereto, of N-terminus; the HPV52 L1 protein may have a deletion of amino acids at positions 2-31, or positions corresponding thereto, of N-terminus.

In another preferred embodiment, the HPV L1 protein may comprise of an amino acid sequence selected from one of SEQ ID NOs: 21-41, or a variant having at least about 90%, or at least about 95%, or at least about 98%, at least about 99% identity to one of SEQ ID NOs: 21-41.

In another preferred embodiment, the HPV L1 protein may consist of an amino acid sequence selected from the group consisting of SEQ ID NOs: 21-41.

In another preferred embodiment, the truncated HPV L1 protein may include a variant in which amino acid sequence is different from the truncated HPV L1 protein according to the disclosure by one or more amino acids, such as conservative amino acid substitutions, or which has an identity of at least about 60%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% to the truncated HPV L1 protein according to the disclosure and retains the essential characteristics of the truncated protein. The term “essential characteristics” may be one or more of the following characteristics: capable of inducing the generation of neutralization antibodies against HPV; capable of being expressed in a yeast cell; capable of obtaining purified protein with a high yield by the expression.

The term “identity” refers to the match degree between two polypeptides or between two nucleic acids. When two sequences for comparison have the same base or amino acid monomer subunit at a certain site (e.g., each of two DNA molecules has an adenine at a certain site, or each of two polypeptides has a lysine at a certain site), the two molecules are identical at the site. Generally, the comparison of two sequences and the percent identity between two amino acid sequences can be determined using a known computer program or algorithm in the art.

The term “conservative substitution” refers to amino acid substitutions which would not negatively affect or change the biological activity of a protein/polypeptide comprising the amino acid sequence. For example, a conservative substitution may be introduced by standard techniques known in the art such as site-directed mutagenesis and PCR-mediated mutagenesis. Conservative amino acid substitutions include substitutions wherein an amino acid residue is substituted with another amino acid residue having a similar side chain, for example, a residue similar to the corresponding amino acid residue biophysically or functionally (such as, having similar size, shape, charges, chemical properties including the capability of forming covalent bond or hydrogen bond, etc.). The families of amino acid residues having similar side chains have been defined in the art. Therefore, a corresponding amino acid residue is preferably substituted with another amino acid residue from the same side-chain family. Methods for identifying amino acid conservative substitutions are well known in the art (for example, Brummell et al., Biochem. 32:1180-1187 (1993); Kobayashi et al., Protein Eng. 12(10): 879-884 (1999); and Burks et al., Proc. Natl Acad. Set USA 94:412-417 (1997), which are incorporated herein by reference).

For non-conservative amino acid modifications, a single amino acid variation in a protein sequence can significantly impact the structural stability, functional activity, or immunogenicity of the protein. In particular, substitution, deletion, or insertion of specific amino acids can alter the secondary and tertiary structures of the protein, thereby affecting expression efficiency, binding affinity, and the ability to induce an immune response. For example, in the major structural protein L1 of the human papillomavirus (HPV) antigen, the deletion of N amino acids at the N-terminus, compared to the deletion of N+1 amino acids, may result in substantial differences in protein expression levels or functional properties. Such variations cannot be predicted solely based on sequence differences and must be experimentally validated to assess their effects. Therefore, in the present invention, the impact of specific amino acid deletions on protein expression and function was experimentally evaluated, and an antigen with an optimal sequence was selected.

In other aspect, the disclosure relates to a recombinant polynucleotide encoding the truncated recombinant HPV L1 protein, a recombinant vector comprising the polynucleotide, an isolated cell comprising the recombinant vector, a composition comprising the protein, and a method for producing the protein in a yeast expression system.

According to the disclosure, the term “yeast expression system” refers to an expression system comprising or consisting of a yeast (strain) and a vector, wherein the yeast (strain) includes, but is not limited to Komagataella phaffii (Pichia pastoris).

It will be appreciated by those skilled in the art that as a result of the degeneracy of the genetic code, each amino acid may be encoded by more than one codon, with the codons for the same amino acid having different frequencies of usage in wild-type genes. For example, yeast cell's preference for codons may result in low translation efficiency and expression levels of recombinant proteins. Thus, it may be advantageous to produce nucleotide sequences encoding proteins possessing a substantially different codon usage. Codons may be selected to tune the rate at which translation of the peptide occurs in a particular host in accordance with the frequency with which particular codons are utilized by the host. Properly tuning the codon usage may lead to improved overall expression.

In this regard, the present disclosure provides a codon-optimized gene encoding the major capsid protein L1 of human papilloma virus, which is capable, when introduced into a yeast cell, of efficiently expressing the major capsid protein L1 of human papilloma virus which then self-assembles into virus-like particles, in an expression amount that meets the requirements of industrial production.

When optimizing a heterologous protein for expression in Komagataella phaffii (Pichia pastoris), it has been reported that the nucleotide sequence may vary depending on the codon optimization strategy employed. Typically, sequence variations range from approximately 10% to 40%, with 20% to 30% being the most commonly reported range in scientific literature (For example, Wang J R et al., Biomed Res Int. 2015:248680).

In a preferred embodiment, the polynucleotide encoding the truncated recombinant HPV L1 protein may be codon-optimized for expression in a yeast cell, preferably Komagataella phaffii. For example, the polynucleotide may consist of a nucleotide sequence selected from the group consisting of SEQ ID NOs: 42-62, 67, and 69-72, but is not limited thereto. For example, the polynucleotide described herein may comprise of a nucleotide sequence selected from one of SEQ ID Nos: 42-62, 67, and 69-72, or a variant having at least about 60%, at least about 70%, at least about 80%, at least about 90%, or at least about 95%, or at least about 98%, at least about 99% identity to one of SEQ ID NOs: 42-62, 67, and 69-72. The nucleotide is suitable to be efficiently expressed in yeast cell, particularly in Komagataella phaffii cell.

The codon-optimized gene provided by the present disclosure has the following advantages: 1) this optimized gene is more suitable for efficiently expressing target protein in yeast host and meets the requirements of industrial production; and 2) low cost, high yield and more uniform and stable quality of products can be achieved with the use of Pichia yeast expression system.

The term “vector” refers to a nucleic acid vehicle which can have a polynucleotide inserted therein. For expression in an appropriate expression system, an L1 nucleic acid encoding a polypeptide is operably linked into an expression vector and introduced into a host cell to enable the expression of the L1 protein by that cell. The gene with the appropriate regulatory regions will be provided in the proper orientation and reading frame to allow for expression. Methods for gene construction are known in the art. For example, in the vector, the polynucleotide may be operably linked to a promoter. The term “operatively linked” refers to a functional linkage between a nucleotide expression control sequence (e.g., a promoter sequence) and another nucleotide sequence. The control sequence may be operably linked to control transcription and/or translation of another nucleotide sequence.

The vector may be constructed as a vector for typically cloning or expression. In the present disclosure, any suitable vector can be used, especially those used for cloning and expression in yeast cell. More preferably, said expression vector is selected from currently commonly used yeast expression vectors, such as pPICZ, pD912, pPIC6, pGAPZ and pAO815, but is not limited thereto. Such expression vectors are commercially available from diverse vendors.

Moreover, an isolated cell comprising said gene encoding HPV L1 is also included in the present disclosure. Said cell is preferably a yeast cell, particularly a Komagataella yeast cell. The yeast cell strains are commercially available.

Delivery (introduction) of the polynucleotide or the vector comprising the same into the host cell may be carried out by using a delivering method widely known in the art. With regard to the delivering method, for example a microinjection method, a calcium phosphate precipitation method, an electroporation method, a liposome-mediated transfection method, and gene bombardment may be used, but is not limited thereto.

A method of selecting the transformed host cell may be easily carried out using a phenotype expressed by a selection marker according to a method well known in the art. For example, when the selection marker is a specific antibiotic resistance gene, the transformant may be easily selected by culturing the transformant in a medium containing the antibiotic. The transformed host cells may be cultured for a period sufficient for polypeptide expression or secretion, and the polypeptide may be separated and purified by a method of separating and purifying proteins commonly used, for example, a method of using solubility, such as salting out, solvent precipitation, etc., a method of using a molecular weight difference, such as dialysis, ultrafiltration, gel filtration, sodium dodecyl sulfate-polyacrylamide gel electrophoresis, etc., a method of using a charge, such as ion exchange chromatography or hydroxylapatite chromatography, etc., a method of using specific affinity, such as affinity chromatography, etc., a method of using a hydrophobicity difference, such as reverse-phase high performance liquid chromatography, etc., a method of using an isoelectric point difference, such as isoelectric point electrophoresis, etc.

In other aspects, the disclosure relates to an HPV virus-like particle (VLP) comprising the protein, an immunogenic composition comprising the protein or the VLP, and a method for inducing an immune response to HPV in an individual.

Some embodiments concern using the protein produced herein for both prophylactic administration to reduce the risk of infection, for treatment of an infection and for diagnostics of an existing condition. Suitability of the protein produced for use as vaccines or as diagnostic agents can be confirmed by reaction with antibodies or monoclonal antibodies that react or recognize conformational epitopes present on the intact version of virus and based on ability to elicit the production of neutralizing antiserum. Other suitable assays for determining whether neutralizing antibodies are produced are known in the art and contemplated herein.

In a preferred embodiment, the HPV L1 protein may be used to generate immunogenic compositions against HPV infection. In accordance with these embodiments, these immunogenic compositions can contain the protein described herein in an effective amount to induce formation of neutralizing antibodies when introduced to a subject in need thereof. Immunogenic or vaccine compositions contemplated herein can further contain a pharmaceutically acceptable carrier or excipients or another anti-viral agent or other known agent in the art to complement the compositions.

An elicited immune response may be either prophylactic, preventing later infection by the specific viral type targeted, or may be therapeutic, reducing the severity of infection or disease. An immune response can include a humoral, e.g., antibody, response to a provided antigen and/or a cell mediated response to a provided antigen in a vaccine. Methods to measure an immune response are known to those skilled in the art. If one or both types of immune response are present, they can protect a subject from developing the condition. In accordance with certain embodiments, ability of a composition disclosed herein to protect from disease refers to the ability of the protein generated herein to treat, ameliorate and/or prevent disease caused by a virus contemplated herein or cross-reactive agent, for example, by eliciting an immune response in the subject. It is to be noted that a subject may be protected by a composition of the present disclosure even without the detection of a humoral or cell-mediated response to the composition. Protection can be measured by methods known to those skilled in the art.

Immunogenic compositions or vaccines can be administered in therapeutically effective amounts. That is, in amounts sufficient to produce a protective immunological response. In accordance with the embodiment, dosages ranging from about 0.001 mg to about 1 mg protein can be introduced to a subject in need thereof or to a subject in order to reduce the onset of a condition associated with infection (e.g. HPV infection). Single or multiple dosages can be administered as determined by a health professional.

The composition may be formulated as either a monovalent or multivalent vaccine. In one embodiment, the composition may comprise the L1 protein from serotypes HPV 6, 11, 18, 16, 31, 33, 35, 45, 52, and 58. For example, the composition may comprise serotypes HPV 6 at 30 μg/human dose, HPV 11 and 18 at 40 μg/human dose, HPV 16 at 60 μg/human dose, and HPV 31, 33, 35, 45, 52, and 58 at 20 μg/human dose of the L1 protein. The preferred human dose is 0.5 mL.

The term “pharmaceutically acceptable carriers and/or excipients” refers to carriers and/or excipients that are pharmacologically and/or physiologically compatible with subjects and active ingredients, and are well known in the art (for example, Remington's Pharmaceutical Sciences. Edited by Gennaro A R, 19th ed. Pennsylvania: Mack Publishing Company, 1995), including, but not limited to pH adjusting agents, surfactants, and ionic strength enhancers. For example, pH adjusting agents include, but are not limited to, phosphate buffers; surfactants include, but are not limited to: anion surfactants, cation surfactants, or non-ionic surfactants (for example, Tween-80); and ionic strength enhancers include, but are not limited to, NaCl.

The term “adjuvant” refers to a substance that is added to the composition to induce, increase, stimulate or strengthen a cellular or humoral immune response to administration of a vaccination described herein. Any adjuvant known in the art that is compatible with compositions disclosed herein is contemplated. Suitable adjuvants include, but are not limited to, other bacterial cell wall components, aluminum-based salts, such as aluminum hydroxide, calcium-based salts, silica, polynucleotides, toxins, such as cholera toxin, toxoids, such as cholera toxoid, serum proteins, other viral coat proteins, other bacterial-derived preparations, block copolymer adjuvants, saponins and their derivatives, and Freund's adjuvants, such as Freund's complete adjuvant. More preferably, the adjuvant may be an aluminum-based adjuvant, such as aluminum hydroxide. The adjuvant is included in an amount effective to stimulate the immune system without causing significant adverse effects. Typical concentrations may range from about 0.001% to 10% by weight of the total composition, although other concentrations may be used as appropriate based on factors such as the type of antigen, the type of adjuvant, the desired immune response, administration route, target population. In a preferred embodiment, the adjuvant may be present at a concentration of about 120 μg/mL.

The composition may be administered parenterally, locally or systemically, including by way of example, oral drops or tablet, intranasal, intravenous, intramuscular, subcutaneous, inhalation, and topical administration. Preferably, it is administered intramuscularly. In one embodiment, the composition was administered intramuscularly in a mouse immunogenicity study. The manner of administration is affected by factors including the natural route of infection. The dosage administered will depend upon factors including the age, health, weight, kind of concurrent treatment, if any, and nature and type of the particular viral, e.g., human papillomavirus. The vaccine or immunogenic composition may be employed in dosage form such as capsules, liquid solutions, suspensions, or elixirs, for oral administration, or sterile liquid formulations such as solutions or suspensions for parenteral or intranasal use.

The composition may be administered in a regimen designed to elicit an optimal immune response. The dosing schedule, including the interval between administrations and the total number of doses, may vary depending on factors such as the type of antigen, the presence of an adjuvant, the route of administration, and the target population. Generally, the composition may be administered in one or multiple doses at appropriate intervals to achieve the desired level of immunity. In a preferred embodiment, the administration occurs twice at an interval of two weeks. In a preferred embodiment suitable for administration to humans, the composition may be administered as two doses at months 0 and 6-12, or alternatively as three doses at months 0, 2, and 6. However, alternative dosing schedules may be used as needed based on clinical considerations and immunogenicity data. Any pharmaceutical formulation known in the art for a vaccine is contemplated herein. It is contemplated that formulations can contain other agents of use in vaccination of a subject including, but not limited to other active or inactive ingredients or compositions known to one skilled in the art.

All contemplated immunogenic or vaccine compositions can be prepared by any method known to one skilled in the art. In certain embodiments, the virus compositions are lyophilized and are mixed with a pharmaceutically acceptable excipient (e.g. water, phosphate buffered saline (PBS), wetting agents etc.). In other embodiments, immunogenic compositions can include stabilizers that are known to reduce degradation of the formulation and prolong shelf-life of the compositions.

### Beneficial Effect

HPV L1 proteins expressed in eukaryotic expression systems retain their native conformation and can self-assemble into VLPs. Nevertheless, low expression levels experienced when using eukaryotes like insect cells limit production at large-scale and result in high costs. The n-truncated HPV L1 protein provided by the present disclosure describes a protein suitable for yeast expression systems with high expression levels. It enables the production of intact VLPs at higher expression levels than those reported for other expression hosts, while increasing production yields and enabling large-scale industrial applications.

### EXAMPLES

Hereinafter, the present disclosure will be described in more detail with reference to the following examples. However, these examples are only for illustrating the present disclosure, and the scope of the present disclosure is not limited by these examples.

### Example 1. Construction of Vector and Yeast Strains for Expression of Mutated HPV L1 Proteins

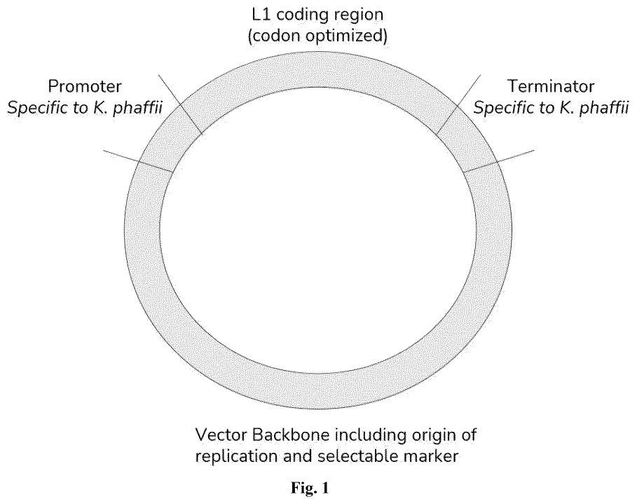

The coding region for L1 proteins of HPV types 6, 11, 16, 18, 31, 33, 35, 45, 52, and 58 (SEQ ID NOs: 1-10) were codon optimized for Komagataella phaffii (Pichia pastoris) (SEQ ID NOs: 11-20) and synthesized by Thermo Fisher GeneArt. The DNA fragment was amplified using PCR and cloned into a custom vector using Gibson Assembly. The custom vector contained a bacterial origin of replication, a promoter specific to Komagataella phaffii, a terminator specific to Komagataella phaffii, and a selectable marker (e.g. antibiotic resistance) (FIG. 1). The circular plasmid containing the HPV L1 coding region and custom vector was transformed into E. coli (TOP10 Chemically Competent E. coli, Thermo Fisher) for amplification. The amplified plasmid was purified from E. coli culture using QIAprep Spin Miniprep Kit (Qiagen) and linearized using PCR. Linearized plasmid was transformed into Komagataella phaffii cells (wild type NRRL Y-11430 retrieved from United States Department of Agriculture (USDA) and Agricultural Research Service (ARS)) via electroporation. Stable Komagataella phaffii transformants were obtained via homologous recombination and selected for on appropriate antibiotic-resistant solid media.

### Example 2. Expression of HPV L1 Proteins in Yeast

Komagataella phaffii strains containing a recombinant HPV L1 gene, such as those constructed in Example 1, were cultivated in 24-well deep well plates (3 mL cultivation volume) at room temperature or in 1 L baffled shake flasks (200 mL cultivation volume) at 25° C. or at 30° C. Strains were cultivated in custom chemically-defined media. Glycerol-containing medium was used for the first 24 hours to build biomass. The medium was then exchanged for a methanol containing medium to promote protein expression. Strains were cultivated in Methanol-containing medium for 24 hours. The yeast cells were then harvested by centrifugation and stored at −80° C. until use. Cell pellets were resuspended in a lysis buffer and lysed with 0.5 mm acid washed Silica glass beads (MilliporeSigma) using a vortexer or a BeadBeater Blender (BioSpec). The lysate was centrifuged at 16,000×g for 30 minutes at 4° C. Cell lysate was sterile filtered using a 0.2 μm filter and stored at −80° C. prior to further purification.

Samples of soluble cell lysate were analyzed for protein expression using SDS-PAGE and Western blot. Samples for SDS-PAGE were reduced using NuPAGE Sample Reducing Agent (Thermo Fisher), boiled at 75° C. for 7 minutes, and run on 12% Tris-Glycine gels (Thermo Fisher). Samples for Western blot were first run on SDS-PAGE and then transferred to a nitrocellulose membrane using an iBlot 2 Dry Blotting System (Thermo Fisher). The membrane was then blocked in TBST-M (Tris-Buffered Saline (TBS, Thermo Fisher) with 0.5% Tween 20 (Sigma) and 5% Skim Milk (Fisher Scientific)) at room temperature for 1 hour, incubated in a solution of primary antibody (Purified mouse Anti-L1 protein of human papilloma virus, BD Bioscience Cat No. 554171) diluted in TBST-M at 4° C. overnight, washed three times with TBST ((Tris-Buffered Saline (TBS, Thermo Fisher) with 0.5% Tween 20 (Sigma)), incubated with secondary antibody (Goat anti-Mouse IgG (H+L) Cross-Adsorbed Secondary Antibody, HRP, Thermo Fisher Cat No. G-21040) diluted in TBST-M at room temperature for 1 hour, washed another three times with TBST, and finally washed once with TBS (Thermo Fisher) for 10 minutes. Western blots were imaged using electrochemiluminescence.

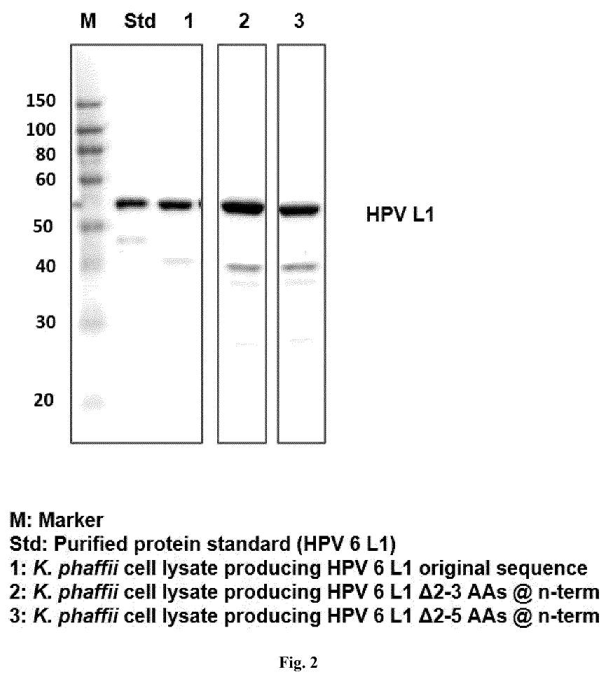

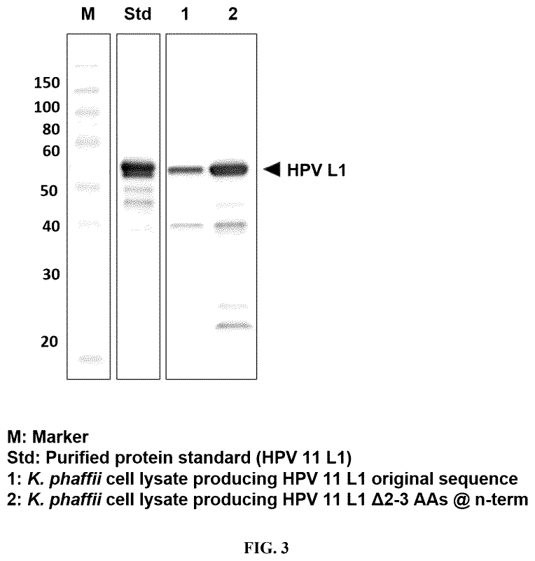

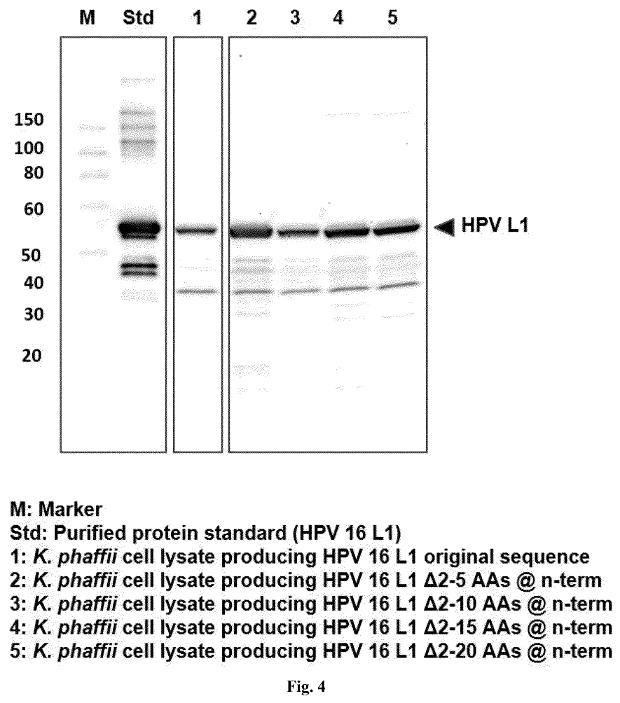

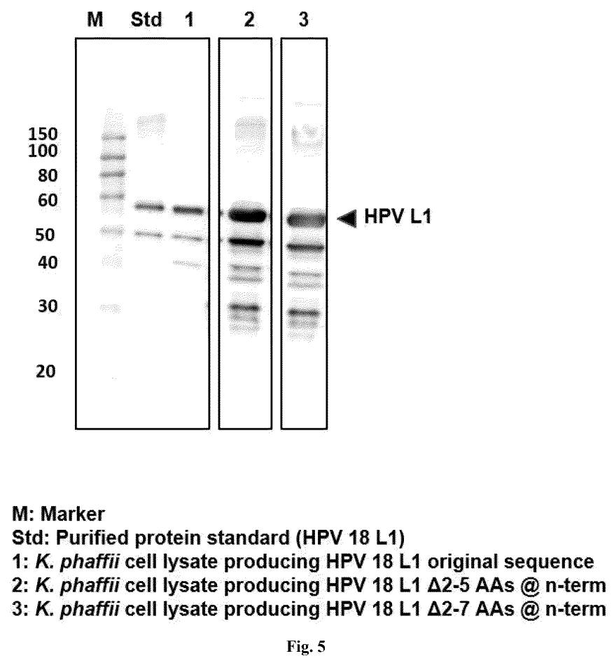

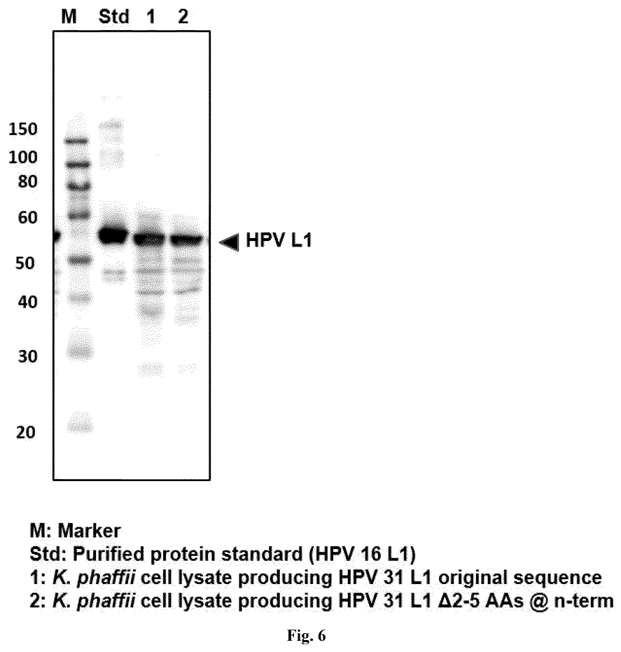

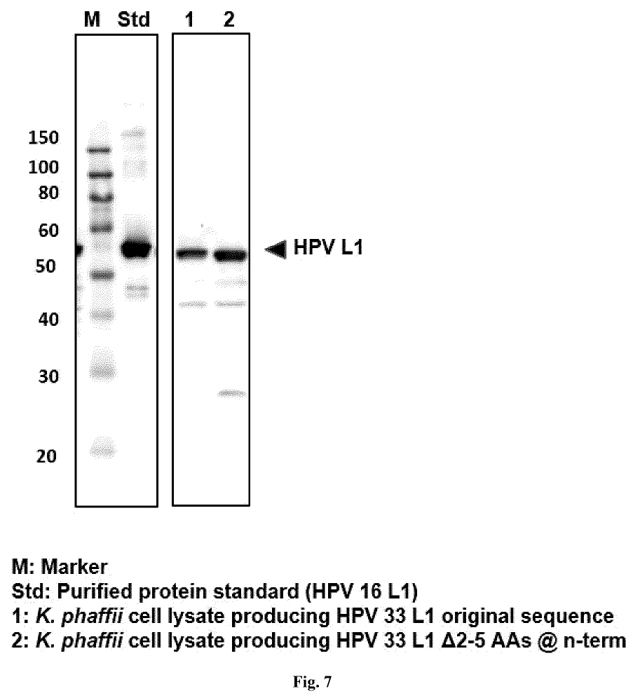

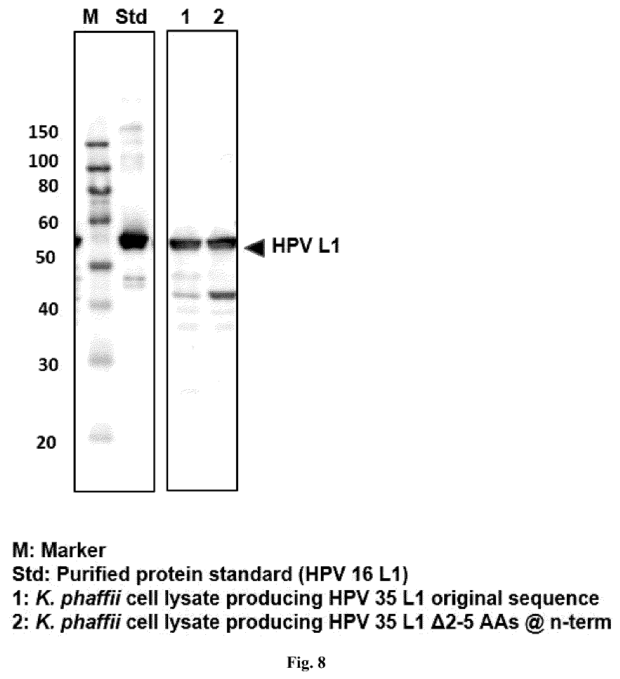

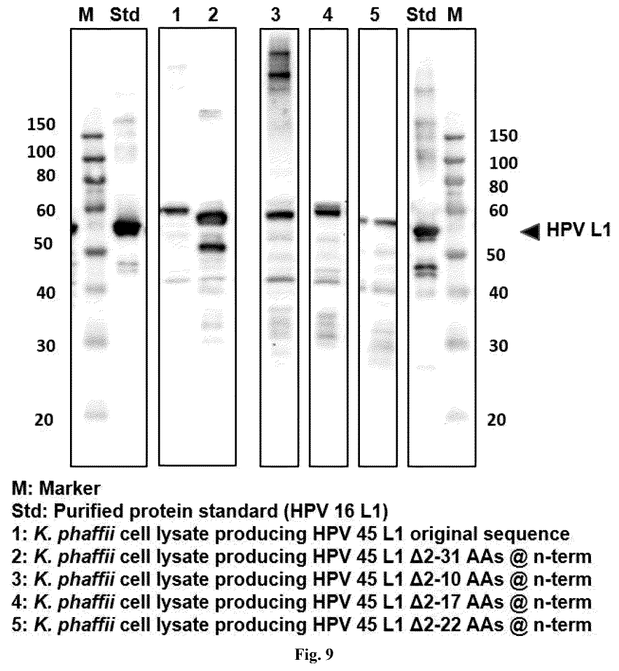

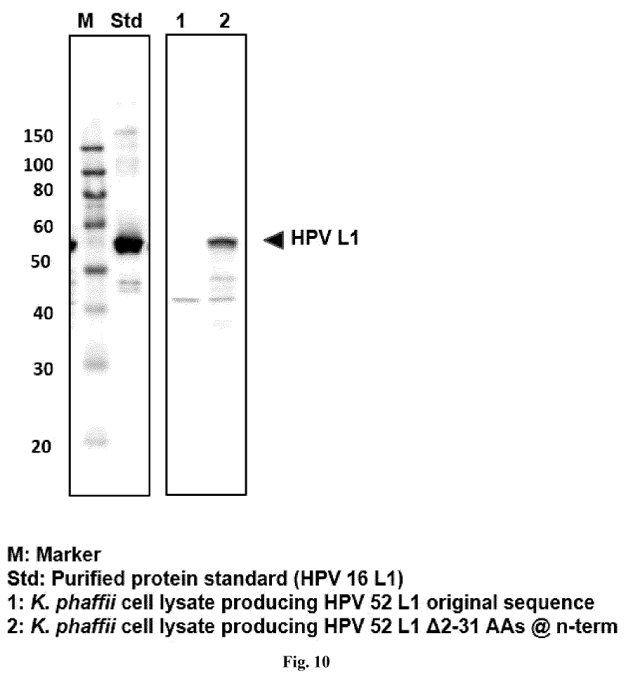

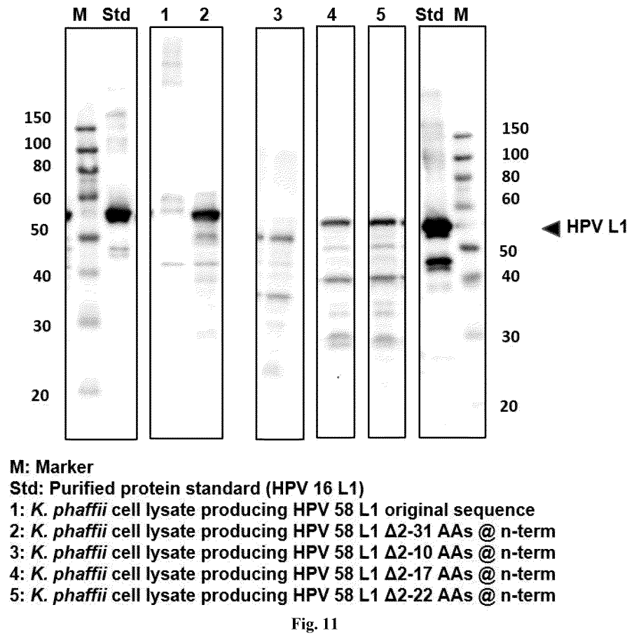

L1 proteins of HPV types 6, 11, 16, 18, 31, 33, 35, 45, 52, and 58 were expressed in Komagataella phaffii as illustrated (FIGS. 2-11; Lane 1).

### Example 3. Purification of Mutated Proteins

The HPV L1 protein produced by Komagataella phaffii was purified by a two-step chromatography method, i.e. HS-HE method, the cell lysate was purified and finally, high-purity virus-like particles were obtained. For the purification step, the supernatant samples were loaded onto a salt-containing buffer (such as NaCl or KCl buffer solution). Then the media was incubated with a commonly used reducing agent and loaded onto the cation exchange chromatography, and then washed using a salt concentration gradient buffer to separate impurities from HPV L1 VLPs protein. Subsequently, a high-concentration salt-containing buffer solution was used to elute the bound HPV L1 VLPs protein, and the eluted preliminarily purified HPV L1 VLPs protein was collected. Thus, the obtained preliminarily purified HPV L1 VLPs protein was once again incubated with a commonly used reducing agent and loaded onto affinity chromatography for fine purification. Similar to the previous step, it was washed using a salt concentration gradient buffer to separate impurities from HPV L1 VLPs protein, and subsequently high concentration salt-containing buffer solution was used to elute the bound HPV L1 VLPs protein, and then the secondly purified HPV L1 VLPs protein was collected. Then tangential flow filtration was applied to the second elution for the final step of the purification and VLP formation of HPV L1 protein. The detailed concentration of salt can vary depending on the serotype of HPV.

### First Step Chromatography:

Resin: POROS™ 50 HS strong cation exchange resin produced by Thermo Fisher was used.

### Second Step Chromatography:

Resin: POROS™ 50 HE Heparin affinity resin produced by Thermo Fisher was used.

### Example 4. Study of Codon Optimization in Intracellular Expression

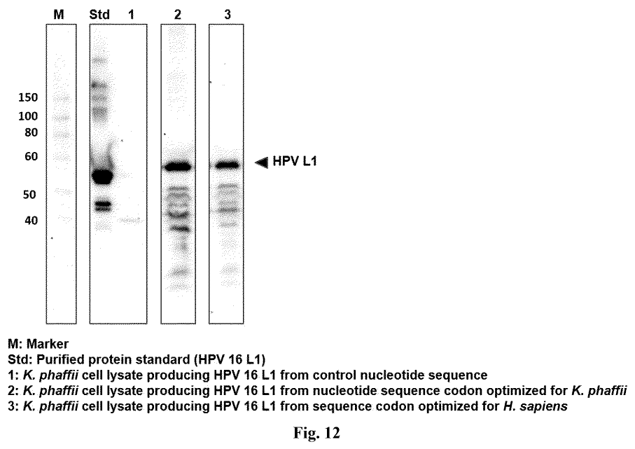

The coding region for L1 protein of HPV type 16 (SEQ ID NO: 2) was codon optimized for Komagataella phaffii (Pichia pastoris) (SEQ ID NO: 12) or for Homo sapiens (SEQ ID NO: 21) and synthesized by Thermo Fisher GeneArt. Yeast (K. phaffii) strains expressing each version of the codon optimized HPV type 16 were constructed as described in Example 1 and expression was evaluated as described in Example 2. Genes that were codon optimized for K. phaffii (SEQ ID NO: 12) or H. sapiens (SEQ ID NO: 64) showed higher expression than the control that was not codon optimized (SEQ ID NO: 74) (FIG. 12).

### Example 5. Evaluating N-Terminal Truncations to Promote HPV L1 Expression

In order to improve the expression of L1 in K. phaffii, a series of deletions were made in the DNA expression plasmids from the 5′-end of the HPV L1 coding regions. Vectors were constructed as in Example 1 for L1 proteins of HPV types 6, 11, 16, 18, 31, 33, 35, 45, 52, and 58 (SEQ ID NOs: 21-41), were codon optimized for Komagataella phaffii (Pichia pastoris) (SEQ ID NOs: 42-62), and were expressed as in Example 2. Proteins with deletions were expressed as illustrated in FIGS. 2-11; Lane 2+. For all serotypes demonstrated, expression of protein with deletion maintained or improved expression levels observed on Western blot (FIGS. 2-11).

### Example 6. Evaluation of Titer According to Amino Acid Changes

Cell lysate samples from Examples 2 and 5 were analyzed via ELISA to confirm function (through binding to relevant antibodies) and quantitatively measure protein expression titer. Briefly, capture antibody was diluted in PBS, loaded into 96-well microtiter plates, and incubated overnight at 4° C. The plate was then washed three times with PBS-T (1×PBS with 0.1% Tween 20) and blocked with 1×PBS with 5% skim milk (blocking buffer) for 4 hours at room temperature. Blocking buffer was then removed and cell lysate samples diluted in PBS with 0.5% skim milk and HPV L1 protein standard diluted in 10 mM sodium phosphate, pH 6.5, 330 mM sodium chloride, 0.01% Tween 80 were added to the plate along with 60 mM sodium phosphate and incubated overnight at room temperature with shaking at 100 rpm. The plate was then washed three times with PBS-T. Detection antibody, diluted in PBS with 0.5% skim milk was added and incubated at room temperature for 1 hour and then washed three times with PBS-T. Streptavidin-HRP was added to the plate and incubated at room temperature for 1 hour. The plate was washed 3 times with PBS-T. TMB substrate was added to the plate and incubated at room temperature for 5 minutes. TMB stop solution was added and OD was measured at 450 nm. Samples were compared to the standards to determine protein titer.

Results showed that expression of N-terminally truncated HPV L1 in yeast was higher than the full length sequence for serotypes 11, 16, 18, 33, 35, 45, 52, and 58 (TABLE 1).

TABLE 1
Expression Titer for HPV L1 in
K. phaffii as Determined by ELISA

| | Amino Acid | SEQ | Estimated Titer |
|---|---|---|---|
| Serotype | Deletions | ID NO: | (mg/L) |
| 11 | None | 10 | 4.51 |
| 11 | Δ2-3 @n-term | 41 | 20.91 |
| 16 | None | 2 | 8.5 |
| 16 | Δ2-5 @n-term | 23 | 21.9 |
| 18 | None | 3 | 1.9 |
| 18 | Δ2-5 @n-term | 27 | 4.5 |
| 33 | None | 5 | 26.8 |
| 33 | Δ2-5 @n-term | 30 | 49.6 |
| 35 | None | 6 | 7.5 |
| 35 | Δ2-5 @n-term | 31 | 15.5 |
| 45 | None | 7 | Below LOD* |
| 45 | Δ2-31 @n-term | 35 | 35.8 |
| 52 | None | 8 | Below LOD |
| 52 | Δ2-31 @n-term | 36 | 68.9 |
| 58 | None | 9 | Below LOD |
| 58 | Δ2-31 @n-term | 40 | 204.2 |

*LOD: Limit of detection

### Example 7. Productivity Improvement at the Bioreactor Level

Komagataella phaffii strains containing a recombinant HPV L1 gene, such as those constructed in Example 1, were inoculated in a Daisy Petal™ perfusion bioreactor at 25° C. or 30° C. Strains were cultivated in custom chemically-defined media. Glycerol-containing medium was fed for the first 40 hours to build biomass. The medium was then changed to a methanol containing medium to promote protein expression. Strains were cultivated in Methanol-containing medium for at least 53 hours. The yeast cells were then harvested by centrifugation and stored at −80° C. until use. Cell pellets were lysed and Western Blots were performed as described in Example 2. ELISA was performed as described in Example 6.

Results showed that expression of N-terminally truncated HPV L1 in yeast was higher than that of the full length sequence for serotypes 11, 16, and 18 (TABLE 2). Productivity was measured as space-time yield, the total mass of protein produced per cultivation volume per cultivation day. The space-time yield for each serotype was normalized to the space-time yield achieved with the full length protein.

TABLE 2
Normalized Space-Time Yield for HPV L1 in K. phaffii in Daisy
Petal ™ perfusion bioreactor as Determined by ELISA

| | Amino Acid | SEQ | Normalized Space-Time |
|---|---|---|---|
| Serotype | Deletions | ID NO: | Yield |
| 11 | None | 10 | 1.0 |
| 11 | Δ2-3 @n-term | 41 | 4.2 |
| 16 | None | 2 | 1.0 |
| 16 | Δ2-5 @n-term | 23 | 5.3 |
| 18 | None | 3 | 1.0 |
| 18 | Δ2-5 @n-term | 27 | 2.6 |

### Example 8. TEM Observation of HPV Virus-Like Particle (VLP)

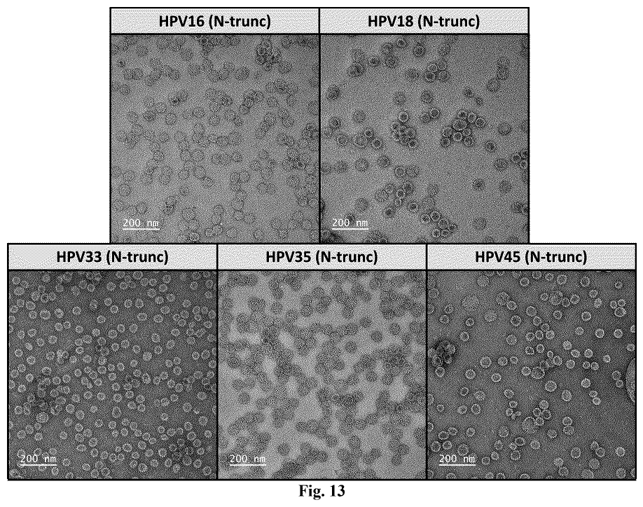

The proper formation of HPV virus-like particles is crucial for the functional aspects of the VLP vaccine. Through the observation using Transmission Electron Microscopy (TEM) of the HPV VLP, we can confirm the formation of VLP with a consistent shape and verify whether the formed VLP exhibits an appropriate structure. Transmission Electron Microscopy involves the convergence of electron beams generated by tungsten filaments, field emission sources, etc., through multiple magnetic lenses. These focused beams pass through the specimen, are detected by a fluorescent screen or CCD detector after passing through several magnetic lenses and are then output. Approximately 20-30 nmol concentration of the sample was dropped onto the plasma-treated carbon grid. To enhance the contrast of the specimen during electron microscopy observation, it was stained with 2% uranyl acetate and then dried. Then the Images were observed using Transmission Electron Microscopy at magnifications of 40,000× and 100,000×. The observation of the shape of the HPV VLP L1 particles formed by HPV16 L1 Δ2-5 through the Transmission Electron Microscopy revealed the consistent formation of VLP of HPV L1 protein (FIG. 13).

### Example 9. Immunogenicity of HPV Virus-Like Particle in Animals

### 9.1 Evaluation of Immunoprotective Characteristics of N-Terminal Truncation on HPV L1 in Animals.

The study compared the immunoprotective characteristics of HPV L1 VLPs made from wildtype HPV16 L1 and an N-terminal truncated HPV16 L1 in animals. The HPV16 Δ2-5 variant was used for the truncated version. The manufactured HPV16 VLPs were adsorbed onto aluminum hydroxide adjuvant at a concentration of 120 μg/mL and diluted 300-fold from the human dose for this animal study. Each experimental group consisted of 7 mice immunized with the diluted antigens. The negative control group of 4 mice received an empty vehicle, while the control group was immunized with an in-house HPV VLP made of wildtype HPV16 L1 expressed in insect cells. Immunizations were administered on Days 0, and 14. Blood samples were collected from the eyes and jugular vein on Day 0, and from the heart on Day 28. Sera were isolated for total antibody and neutralizing antibody titration. Blood samples were incubated at room temperature for 30 minutes for coagulation, then centrifuged for 10 minutes at 2,000 g to separate sera. The supernatants were used for total antibody and neutralizing antibody titration.

TABLE 3

| Test substance | Antigen content μg/human dose |
|---|---|
| Vehicle | 0 |
| In-house Control (WT, Insect) | 60 |
| HPV16 mono (WT, Yeast) | 60 |
| HPV16 mono (N-trunc, Yeast) | 60 |

To evaluate the immunogenicity of the injected HPV VLPs, VLP based Enzyme-Linked Immunosorbent Assay (VLP ELISA) was used to titrate total antibodies, and Pseudovirus-based Neutralization Assay (PBNA) was used to titrate neutralizing antibodies in the immunized mice sera. For VLP ELISA, purified VLPs were coated on immune plates a day before the assay. After removing unbound VLPs, the plates were incubated with skim milk blocking buffer. Serially diluted mice sera, including negative and positive control groups, were then incubated. Anti-mouse IgG HRP was added for signal development, and signals were quantified by a microplate reader at 450 nm after the TMB substrate/stop solution was added.

PBNA, which has replaced traditional immunogenicity tests for HPV due to its difficulty to culture in vitro and strong host specificity, uses HPV pseudovirus composed of major capsid proteins L1 and L2 to mimic the immunogenic characteristics of natural HPV VLPs. By incubating the serum of immunized mice with HPV pseudovirus expressing SEAP (Secreted Alkaline Phosphatase) reporter system, the strength of immunogenicity can be measured quantitatively. 293TT cells were inoculated on 96-well plates at a density of 30,000 cells/well and incubated at 37° C. with 5% CO₂. Serially diluted mice sera, including negative and control groups, were mixed with appropriate subtypes of HPV pseudovirus and incubated at 4° C. for 1 hour. The sera and pseudovirus mixtures were added to inoculated 293TT cells and incubated for 4 days. The chemifluorescence of the culture media was measured by a fluorescence plate reader. Dose-response curves were generated for each experimental group based on the signals produced by the serially diluted sera in VLP ELISA and PBNA. The specific dilution factors where the signal reached 50% of the maximum signal of the curves were designated as EC₅₀ for total antibody titration and neutralizing antibodies. Relative potency of each group was determined by normalizing each EC₅₀ value to the EC₅₀ of the control group.

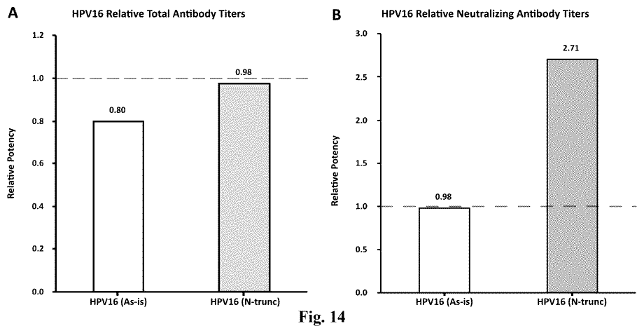

While the HPV16 truncated L1 VLP injected mice sera showed nearly identical total antibody titers to the control group (wildtype HPV16 L1 expressed in insect cells), the wildtype HPV16 was 20% lower in total antibody relative potency compared to the control group (FIG. 14A). Although the wildtype HPV16 L1 VLP showed relatively identical neutralizing antibody relative potency to the control group, the truncated variant showed about a 2.71-fold increase in relative potency (FIG. 14B).

### 9.2 Evaluation of Immunoprotective Characteristics of N-Terminal Truncated HPV L1 VLPs in Multivalent Forms in Animals.

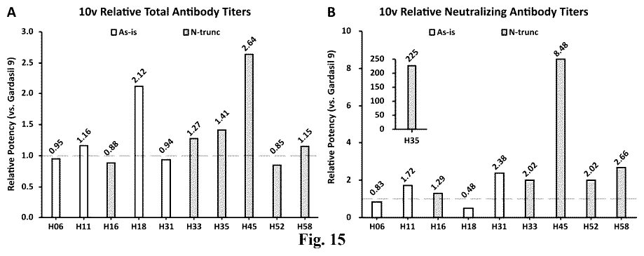

Another study was conducted to examine the immunoprotective characteristics of truncated HPV L1 VLPs when formulated into a multivalent form. For this study, the following HPV types were selected based on manufacturing feasibility: As-is HPV6, HPV11, HPV18, HPV31 (denoted as H06, H11, H18, and H31 in FIG. 15, respectively), and N-terminal truncated HPV16 Δ2-5, HPV33 Δ2-5, HPV35 Δ2-5, HPV45 Δ2-31, HPV52 Δ2-31, and HPV58 Δ2-31 (denoted as H16, H33, H35, H45, H52, and H58 in FIG. 15, respectively). The manufactured HPV VLPs were adsorbed onto aluminum phosphate adjuvant and formulated into a decavalent suspension with the corresponding concentrations for each antigen listed in Table 4. The decavalent suspension was diluted 300-fold from the human dose for this animal study. Both the control and experimental groups consisted of 7 mice immunized with the diluted antigens. For the control group, Gardasil®9, a commercially available nonavalent product, was diluted 300-fold from the human dose. The negative control group of 3 mice received an empty vehicle. Immunizations and analyses were performed as described previously.

TABLE 4

| Test substance | Antigen content μg/human dose (serotype) |
|---|---|
| Vehicle | 0 |
| Control | 30 (6), 40 (11, 18), 60 (16), 20 (31, 33, 45, 52, 58) |
| (Gardasil ®9) | |
| 10v HPV VLPs | 30 (6), 40 (11, 18), 60 (16), 20 (31, 33, 35, 45, 52, 58) |

The relative total antibody titers appeared to be unaffected by the N-terminal truncation, as both the As-is and N-terminal truncated subtypes showed either nearly identical or superior relative potency compared to the control group (FIG. 15A). However, all N-terminal truncated subtypes exhibited at least a 29% increase in relative neutralizing antibody titers compared to the control group, while some As-is subtypes showed up to a 52% decrease in relative potency (FIG. 15B).

Based on the above description, it will be understood by those skilled in the art that the present disclosure may be implemented in a different specific form without changing the technical spirit or essential characteristics thereof. In this regard, it should be understood that the above embodiment is not limitative, but illustrative in all aspects. The scope of the disclosure is defined by the appended claims rather than by the description preceding them, and therefore all changes and modifications that fall within metes and bounds of the claims, or equivalents of such metes and bounds, are intended to be embraced by the claims.

### SEQUENCE INFORMATION

| | | |
|---|---|---|
| SEQ | | |
| ID NO: | type | Info. |

As-Is Protein Sequences

| | | |
|---|---|---|
| 1 | P | HPV6 |
| 2 | P | HPV16 |
| 3 | P | HPV18 |
| 4 | P | HPV31 |
| 5 | P | HPV33 |
| 6 | P | HPV35 |
| 7 | P | HPV45 |
| 8 | P | HPV52 |
| 9 | P | HPV58 |
| 10 | P | HPV11 |

As-Is Nucleotide Sequences codon optimized for K. phaffii

| | | |
|---|---|---|
| 11 | N | HPV6 (codon optimized for K. phaffii) |
| 12 | N | HPV16 (codon optimized for K. phaffii) |
| 13 | N | HPV18 (codon optimized for K. phaffii) |
| 14 | N | HPV31 (codon optimized for K. phaffii) |
| 15 | N | HPV33 (codon optimized for K. phaffii) |
| 16 | N | HPV35 (codon optimized for K. phaffii) |
| 17 | N | HPV45 (codon optimized for K. phaffii) |
| 18 | N | HPV52 (codon optimized for K. phaffii) |
| 19 | N | HPV58 (codon optimized for K. phaffii) |
| 20 | N | HPV11 (codon optimized for K. phaffii) |

N-truncated Protein Sequences

| | | |
|---|---|---|
| 21 | P | HPV6 Δ2-3@n-term |
| 22 | P | HPV6 Δ2-5@n-term |
| 23 | P | HPV16 Δ2-5@n-term |
| 24 | P | HPV16 Δ2-10@n-term |
| 25 | P | HPV16 Δ2-15@n-term |
| 26 | P | HPV16 Δ2-20@n-term |
| 27 | P | HPV18 Δ2-5@n-term |
| 28 | P | HPV18 Δ2-7@n-term |
| 29 | P | HPV31 Δ2-5@n-term |
| 30 | P | HPV33 Δ2-5@n-term |
| 31 | P | HPV35 Δ2-5@n-term |
| 32 | P | HPV45 Δ2-10@n-term |
| 33 | P | HPV45 Δ2-17@n-term |
| 34 | P | HPV45 Δ2-22@n-term |
| 35 | P | HPV45 Δ2-31@n-term |
| 36 | P | HPV52 Δ2-31@n-term |
| 37 | P | HPV58 Δ2-10@n-term |
| 38 | P | HPV58 Δ2-17@n-term |
| 39 | P | HPV58 Δ2-22@n-term |
| 40 | P | HPV58 Δ2-31@n-term |
| 41 | P | HPV11 Δ2-3@n-term |

N-truncated Nucleotide Sequences codon optimized for K. phaffii

| | | |
|---|---|---|
| 42 | N | HPV6 Δ2-3@n-term (codon optimized for K. phaffii) |
| 43 | N | HPV6 Δ2-5@n-term (codon optimized for K. phaffii) |
| 44 | N | HPV16 Δ2-5@n-term (codon optimized for K. phaffii) |
| 45 | N | HPV16 Δ2-10@n-term (codon optimized for K. phaffii) |
| 46 | N | HPV16 Δ2-15@n-term (codon optimized for K. phaffii) |
| 47 | N | HPV16 Δ2-20@n-term (codon optimized for K. phaffii) |
| 48 | N | HPV18 Δ2-5@n-term (codon optimized for K. phaffii) |
| 49 | N | HPV18 Δ2-7@n-term (codon optimized for K. phaffii) |
| 50 | N | HPV31 Δ2-5@n-term (codon optimized for K. phaffii) |
| 51 | N | HPV33 Δ2-5@n-term (codon optimized for K. phaffii) |
| 52 | N | HPV35 Δ2-5@n-term (codon optimized for K. phaffii) |
| 53 | N | HPV45 Δ2-10@n-term (codon optimized for K. phaffii) |
| 54 | N | HPV45 Δ2-17@n-term (codon optimized for K. phaffii) |
| 55 | N | HPV45 Δ2-22@n-term (codon optimized for K. phaffii) |
| 56 | N | HPV45 Δ2-31@n-term (codon optimized for K. phaffii) |
| 57 | N | HPV52 Δ2-31@n-term (codon optimized for K. phaffii) |
| 58 | N | HPV58 Δ2-10@n-term (codon optimized for K. phaffii) |
| 59 | N | HPV58 Δ2-17@n-term (codon optimized for K. phaffii) |
| 60 | N | HPV58 Δ2-22@n-term (codon optimized for K. phaffii) |
| 61 | N | HPV58 Δ2-31@n-term (codon optimized for K. phaffii) |
| 62 | N | HPV11 Δ2-3 @n-term (codon optimized for K. phaffii) |

As-Is Nucleotide Sequences codon optimized for H. sapiens

| | | |
|---|---|---|
| 63 | N | HPV6 (codon optimized for H. sapiens) |
| 64 | N | HPV16 (codon optimized for H. sapiens) |
| 65 | N | HPV18 (codon optimized for H. sapiens) |
| 66 | N | HPV31 (codon optimized for H. sapiens) |
| 67 | N | HPV33 Δ2-5@n-term (codon optimized for H. sapiens) |
| 68 | N | HPV35 (codon optimized for H. sapiens) |
| 69 | N | HPV35 Δ2-5 @n-term(codon optimized for H. sapiens) |
| 70 | N | HPV45 Δ2-31 @n-term(codon optimized for H. sapiens) |
| 71 | N | HPV52 Δ2-31 @n-term (codon optimized for H. sapiens) |
| 72 | N | HPV58 Δ2-31 @n-term (codon optimized for H. sapiens) |
| 73 | N | HPV6 |
| 74 | N | HPV16 |
| 75 | N | HPV18 Δ2-66 @n-term |
| 76 | N | HPV31 |
| 77 | N | HPV33 Δ2-5 @n-term |
| 78 | N | HPV35 |
| 79 | N | HPV35 Δ2-5 @n-term |
| 80 | N | HPV45 Δ2-31 @n-term |
| 81 | N | HPV52 Δ2-31 @n-term |
| 82 | N | HPV58 Δ2-31 @n-term |

1. A truncated recombinant human papillomavirus (HPV) L1 protein with at least 2 amino acids deleted from positions 2 to 66, or positions corresponding thereto, of the N-terminus.

2. The protein according to claim 1, wherein the HPV L1 protein has a deletion of amino acids at positions 2-3, 2-5, 2-7, 2-10, 2-15, 2-17, 2-20, 2-22, 2-31, or 2-66, or positions corresponding thereto, of the N-terminus.

3. The protein according to claim 1, wherein the HPV is selected from HPV serotypes 6, 11, 16, 18, 31, 33, 35, 45, 52, and 58.

4. The protein according to claim 3,
wherein the HPV6 L1 protein has a deletion of amino acids at positions 2-3, or positions corresponding thereto, of the N-terminus relative to SEQ ID NO: 1;
wherein the HPV11 L1 protein has a deletion of amino acids at positions 2-3, or positions corresponding thereto, of the N-terminus relative to SEQ ID NO: 10;
wherein the HPV16 L1 protein has a deletion of amino acids at positions 2-5, 2-10, 2-15, or 2-20, or positions corresponding thereto, of the N-terminus relative to SEQ ID NO: 2;
wherein the HPV18 L1 protein has a deletion of amino acids at positions 2-5, 2-7, or 2-66, or positions corresponding thereto, of the N-terminus relative to SEQ ID NO: 3;
wherein the HPV31, HPV33, or HPV35 L1 protein has a deletion of amino acids at positions 2-5, or positions corresponding thereto, of the N-terminus relative to SEQ ID NOs: 4, 5, or 6;
wherein the HPV45 or HPV58 L1 protein has a deletion of amino acids at positions 2-10, 2-17, 2-22, or 2-31, or positions corresponding thereto, of the N-terminus relative to SEQ ID NOs: 7 or 9; or
wherein the HPV52 L1 protein has a deletion of amino acids at positions 2-31, or positions corresponding thereto, of the N-terminus relative to SEQ ID NO: 8.

5. The protein according to claim 1, wherein the HPV L1 protein comprises or consists of an amino acid sequence selected from one of SEQ ID NOs: 21-41, or a variant having at least about 90%, or at least about 95%, or at least about 98%, at least about 99% identity to one of SEQ ID NOs: 21-41.

6. A recombinant polynucleotide encoding the protein according to claim 1.

7. The polynucleotide of claim 6, comprising or consisting of a nucleotide sequence selected from one of SEQ ID NOs: 42-62, or a variant having at least about 60%, at least about 70%, at least about 80%, at least about 90%, or at least about 95%, or at least about 98%, at least about 99% identity to one of SEQ ID NOs: 42-62.

8. A recombinant vector comprising the polynucleotide according to claim 6.

9. An isolated cell comprising the recombinant vector according to claim 8.

10. A composition comprising the protein according to claim 1.

11. A recombinant human papillomavirus (HPV) virus-like particle (VLP) comprising the protein of claim 1.

12. An immunogenic composition comprising the protein, or a virus-like particle (VLP) made therefrom, according to claim 1, and one or more pharmaceutically acceptable carriers, excipients and/or adjuvants.

13. The composition of claim 12, wherein the composition comprises serotypes HPV 6, 11, 18, 16, 31, 33, 35, 45, 52, and 58 of the L1 protein.

14. The composition of claim 13, wherein the composition comprises serotypes HPV 6 at 30 μg/human dose, HPV 11 and 18 at 40 μg/human dose, HPV 16 at 60 μg/human dose, and HPV 31, 33, 35, 45, 52, and 58 at 20 μg/human dose of the L1 protein.

15. The immunogenic composition of claim 12, wherein the adjuvant is aluminum hydroxide adjuvant.

16. The immunogenic composition of claim 14, wherein the immunogenic composition is formulated for the prevention of HPV infection or a disease associated with an HPV infection.

17. A method for inducing an immune response to HPV in an individual, comprising administering the immunogenic composition according to claim 14 to an individual.

18. A method for preventing an HPV infection or a disease associated with an HPV infection, comprising administering the immunogenic composition of claim 14 to a subject in need thereof.

19. The method of claim 18, wherein the administration occurs twice at an interval of two weeks.

20. A method for producing the HPV L1 protein according to claim 1, comprising expressing a polynucleotide encoding the HPV L1 protein in a yeast cell.
